SEQUENCE LISTING (65 sequences)

SEQ ID NO: 1 - protein, 12 aa, synthetic construct
  REGION 1..12: note = Synthetic peptide
AQNDTSQTSSPS

SEQ ID NO: 2 - protein, 12 aa, synthetic construct
  REGION 1..12: note = Synthetic peptide
GANDTSQTSSPS

SEQ ID NO: 3 - protein, 12 aa, synthetic construct
  REGION 1..12: note = Synthetic peptide
GQADTSQTSSPS

SEQ ID NO: 4 - protein, 12 aa, synthetic construct
  REGION 1..12: note = Synthetic peptide
GQNATSQTSSPS

SEQ ID NO: 5 - protein, 12 aa, synthetic construct
  REGION 1..12: note = Synthetic peptide
GQNDASQTSSPS

SEQ ID NO: 6 - protein, 12 aa, synthetic construct
  REGION 1..12: note = Synthetic peptide
GQNDTAQTSSPS

SEQ ID NO: 7 - protein, 12 aa, synthetic construct
  REGION 1..12: note = Synthetic peptide
GQNDTSATSSPS

SEQ ID NO: 8 - protein, 12 aa, synthetic construct
  REGION 1..12: note = Synthetic peptide
GQNDTSQASSPS

SEQ ID NO: 9 - protein, 12 aa, synthetic construct
  REGION 1..12: note = Synthetic peptide
GQNDTSQTASPS

SEQ ID NO: 10 - protein, 12 aa, synthetic construct
  REGION 1..12: note = Synthetic peptide
GQNDTSQTSAPS

SEQ ID NO: 11 - protein, 12 aa, synthetic construct
  REGION 1..12: note = Synthetic peptide
GQNDTSQTSSPS

SEQ ID NO: 12 - protein, 12 aa, synthetic construct
  REGION 1..12: note = Synthetic peptide
GQNDTSQAASPS

SEQ ID NO: 13 - protein, 12 aa, synthetic construct
  REGION 1..12: note = Synthetic peptide
GQNDTSQASAPS

SEQ ID NO: 14 - protein, 12 aa, synthetic construct
  REGION 1..12: note = Synthetic peptide
GQNDTSQAAAPS

SEQ ID NO: 15 - protein, 12 aa, synthetic construct
  REGION 1..12: note = Synthetic peptide
GANDTSQAASPS

SEQ ID NO: 16 - protein, 12 aa, synthetic construct
  REGION 1..12: note = Synthetic peptide
GANDTSQASAPS

SEQ ID NO: 17 - protein, 12 aa, synthetic construct
  REGION 1..12: note = Synthetic peptide
GQNDTSAAASPS

SEQ ID NO: 18 - protein, 12 aa, synthetic construct
  REGION 1..12: note = Synthetic peptide
GQNDTSAASAPS

SEQ ID NO: 19 - protein, 12 aa, synthetic construct
  REGION 1..12: note = Synthetic peptide
  SITE 2: note = misc_feature - Xaa can be any naturally occurring amino acid
GXNDTSQTSSPS

SEQ ID NO: 20 - protein, 12 aa, synthetic construct
  REGION 1..12: note = Synthetic peptide
  SITE 4: note = misc_feature - Xaa can be any naturally occurring amino acid
GQNXTSQTSSPS

SEQ ID NO: 21 - protein, 12 aa, synthetic construct
  REGION 1..12: note = Synthetic peptide
  SITE 7: note = misc_feature - Xaa can be any naturally occurring amino acid
GQNDTSXTSSPS

SEQ ID NO: 22 - protein, 12 aa, synthetic construct
  REGION 1..12: note = Synthetic peptide
  SITE 8: note = misc_feature - Xaa can be any naturally occurring amino acid
GQNDTSQXSSPS

SEQ ID NO: 23 - protein, 12 aa, synthetic construct
  REGION 1..12: note = Synthetic peptide
  REGION 8..9: note = misc_feature - Xaa can be any naturally occurring amino acid
GQNDTSQXXSPS

SEQ ID NO: 24 - protein, 12 aa, synthetic construct
  REGION 1..12: note = Synthetic peptide
  SITE 8: note = misc_feature - Xaa can be any naturally occurring amino acid
  SITE 10: note = misc_feature - Xaa can be any naturally occurring amino acid
GQNDTSQXSXPS

SEQ ID NO: 25 - protein, 12 aa, synthetic construct
  REGION 1..12: note = Synthetic peptide
  REGION 8..10: note = misc_feature - Xaa can be any naturally occurring amino acid
GQNDTSQXXXPS

SEQ ID NO: 26 - protein, 12 aa, synthetic construct
  REGION 1..12: note = Synthetic peptide
  SITE 2: note = misc_feature - Xaa can be any naturally occurring amino acid
  REGION 8..9: note = misc_feature - Xaa can be any naturally occurring amino acid
GXNDTSQXXSPS

SEQ ID NO: 27 - protein, 12 aa, synthetic construct
  REGION 1..12: note = Synthetic peptide
  SITE 2: note = misc_feature - Xaa can be any naturally occurring amino acid
  SITE 8: note = misc_feature - Xaa can be any naturally occurring amino acid
  SITE 10: note = misc_feature - Xaa can be any naturally occurring amino acid
GXNDTSQXSXPS

SEQ ID NO: 28 - protein, 12 aa, synthetic construct
  REGION 1..12: note = Synthetic peptide
  REGION 7..9: note = misc_feature - Xaa can be any naturally occurring amino acid
GQNDTSXXXSPS

SEQ ID NO: 29 - protein, 12 aa, synthetic construct
  REGION 1..12: note = Synthetic peptide
  REGION 7..8: note = misc_feature - Xaa can be any naturally occurring amino acid
  SITE 10: note = misc_feature - Xaa can be any naturally occurring amino acid
GQNDTSXXSXPS

SEQ ID NO: 30 - DNA, 189 bp, synthetic construct
  misc_feature 1..189: note = Synthetic polynucleotide
  source 1..189: mol_type = other DNA
aaaatgaagcgcttcctcttcctcctactcaccatcagcctcctggttatggtacagatacaaactggactctcaggacaaaacgacaccagccaaaccagcagcccctcagcatccagcaacataagcggaggcattttccttttcttcgtggccaatgccataatccacctcttctgcttcagttga

SEQ ID NO: 31 - DNA, 227 bp, synthetic construct
  misc_feature 1..227: note = Synthetic polynucleotide
  source 1..227: mol_type = other DNA
accttggagcgcttcctcttcctcctactcaccatcagcctcctcgttttggtacaaatacaaaccggactctccggacaaaacgccaccagccaaaccagcagcccctcagcatccagcaacataagcggaggcattttccttttcttcgtcgccaacgccataatccacctcttctgcttcagttgaaggccggccaatacgtaggcgcgccattgagtgagtga

SEQ ID NO: 32 - DNA, 227 bp, synthetic construct
  misc_feature 1..227: note = Synthetic polynucleotide
  source 1..227: mol_type = other DNA
accttggagcgcttcctcttcctcctactcaccatcagcctcctcgttttggtacaaatacaaaccggactctcaggacaaaacgacaccagccaagccagcagcccctcagcatccagcaacataagcggaggcattttccttttcttcgtcgccaacgccataatccacctcttctgcttcagttgaaggccggccaatacgtaggcgcgccattgagtgagtga

SEQ ID NO: 33 - DNA, 227 bp, synthetic construct
  misc_feature 1..227: note = Synthetic polynucleotide
  source 1..227: mol_type = other DNA
accttggagcgcttcctcttcctcctactcaccatcagcctcctcgttttggtacaaatacaaaccggactctcaggacaaaacgacaccagccaagccagcgccccctcagcatccagcaacataagcggaggcattttccttttcttcgtcgccaacgccataatccacctcttctgcttcagttgaaggccggccaatacgtaggcgcgccattgagtgagtga

SEQ ID NO: 34 - DNA, 10 bp, synthetic construct
  misc_feature 1..10: note = Synthetic polynucleotide
  source 1..10: mol_type = other DNA
gccrccatgg

SEQ ID NO: 35-38 - skipped (4 entries)

SEQ ID NO: 39 - protein, 14 aa, synthetic construct [sequence omitted]

SEQ ID NO: 40 - protein, 13 aa, synthetic construct
  REGION 1..13: note = Synthetic peptide
QNDTSQTSSPSAD

SEQ ID NO: 41 - protein, 12 aa, synthetic construct
  REGION 1..12: note = Synthetic peptide
NDTSQTSSPSAD

SEQ ID NO: 42 - protein, 11 aa, synthetic construct
  REGION 1..11: note = Synthetic peptide
DTSQTSSPSAD

SEQ ID NO: 43 - protein, 10 aa, synthetic construct
  REGION 1..10: note = Synthetic peptide
TSQTSSPSAD

SEQ ID NO: 44 - protein, 9 aa, synthetic construct
  REGION 1..9: note = Synthetic peptide
SQTSSPSAD

SEQ ID NO: 45 - protein, 8 aa, synthetic construct
  REGION 1..8: note = Synthetic peptide
QTSSPSAD

SEQ ID NO: 46 - protein, 7 aa, synthetic construct
  REGION 1..7: note = Synthetic peptide
TSSPSAD

SEQ ID NO: 47 - protein, 13 aa, synthetic construct
  REGION 1..13: note = Synthetic peptide
GQNDTSQTSSPAD

SEQ ID NO: 48 - protein, 12 aa, synthetic construct
  REGION 1..12: note = Synthetic peptide
GQNDTSQTSSAD

SEQ ID NO: 49 - protein, 11 aa, synthetic construct
  REGION 1..11: note = Synthetic peptide
GQNDTSQTSAD

SEQ ID NO: 50 - protein, 10 aa, synthetic construct
  REGION 1..10: note = Synthetic peptide
GQNDTSQTAD

SEQ ID NO: 51 - protein, 9 aa, synthetic construct
  REGION 1..9: note = Synthetic peptide
GQNDTSQAD

SEQ ID NO: 52 - protein, 8 aa, synthetic construct
  REGION 1..8: note = Synthetic peptide
GQNDTSAD

SEQ ID NO: 53 - protein, 7 aa, synthetic construct
  REGION 1..7: note = Synthetic peptide
GQNDTAD

SEQ ID NO: 54 - protein, 14 aa, synthetic construct
  REGION 1..14: note = Synthetic peptide
AQNDTSQTSSPSAD

SEQ ID NO: 55 - protein, 14 aa, synthetic construct
  REGION 1..14: note = Synthetic peptide
GANDTSQTSSPSAD

SEQ ID NO: 56 - protein, 14 aa, synthetic construct
  REGION 1..14: note = Synthetic peptide
GQADTSQTSSPSAD

SEQ ID NO: 57 - protein, 14 aa, synthetic construct
  REGION 1..14: note = Synthetic peptide
GQNATSQTSSPSAD

SEQ ID NO: 58 - protein, 14 aa, synthetic construct
  REGION 1..14: note = Synthetic peptide
GQNDASQTSSPSAD

SEQ ID NO: 59 - protein, 14 aa, synthetic construct
  REGION 1..14: note = Synthetic peptide
GQNDTAQTSSPSAD

SEQ ID NO: 60 - protein, 14 aa, synthetic construct
  REGION 1..14: note = Synthetic peptide
GQNDTSATSSPSAD

SEQ ID NO: 61 - protein, 14 aa, synthetic construct
  REGION 1..14: note = Synthetic peptide
GQNDTSQASSPSAD

SEQ ID NO: 62 - protein, 14 aa, synthetic construct
  REGION 1..14: note = Synthetic peptide
GQNDTSQTASPSAD

SEQ ID NO: 63 - protein, 14 aa, synthetic construct
  REGION 1..14: note = Synthetic peptide
GQNDTSQTSAPSAD

SEQ ID NO: 64 - protein, 14 aa, synthetic construct
  REGION 1..14: note = Synthetic peptide
GQNDTSQTSSASAD

SEQ ID NO: 65 - protein, 14 aa, synthetic construct
  REGION 1..14: note = Synthetic peptide
GQNDTSQTSSPAAD

(19) United States
(12) Patent Application Publication
(10) Pub. No.: US 2024/0035022 A1
(43) Pub. Date: Feb. 1, 2024

(54) FLOW CYTOMETRY ATTENUATED REPORTER EXPRESSION (FLARE) MULTIPLE REPORTER SYSTEM AND METHODS OF USE THEREOF

(71) Applicant: Genzyme Corporation, Cambridge, MA (US)

(72) Inventors: Victor R. Cairns, Cambridge, MA (US); David Reczek, Cambridge, MA (US); Jason Vitko, Cambridge, MA (US)

(21) Appl. No.: 18/315,316

(22) Filed: May 10, 2023

Related U.S. Application Data
(63) Continuation of application No. PCT/US2021/059946, filed on Nov. 18, 2021.
(60) Provisional application No. 63/116,094, filed on Nov. 19, 2020.

(51) Int. Cl. C12N 15/10

(52) U.S. Cl. CPC C12N 15/1086

(57) ABSTRACT

Provided are compositions and methods for expressing a plurality of target polypeptides at a high level in mammalian host cells. Each target polypeptide is encoded on a polynucleotide comprising a nucleotide sequence encoding a unique cell surface marker polypeptide and a nucleotide sequence encoding a unique target polypeptide, wherein both the nucleotide sequence encoding the unique cell surface marker polypeptide and the nucleotide sequence encoding the unique target polypeptide are transcribed on the same mRNA. Each mammalian host cell comprises a plurality of different polynucleotides, such that the cell is capable of expressing a plurality of unique target polypeptides and a plurality of unique cell surface marker polypeptides. In certain embodiments, the plurality of unique cell surface marker polypeptides are variants of CD52. The compositions and methods are useful for expressing multimeric target proteins, e.g., crossover dual-variable domain (CODV) triabodies.

### RELATED APPLICATIONS

This application is a continuation of International Patent Application No. PCT/US2021/059946, filed Nov. 18, 2021, which claims the benefit of U.S. Provisional Patent Application Ser. No. 63/116,094, filed Nov. 19, 2020, the entire contents of which are incorporated herein by reference.

### SEQUENCE LISTING

The instant application contains a Sequence Listing which has been submitted electronically in XML format and is hereby incorporated by reference in its entirety. Said XML file, created on May 10, 2023, is named 743135_SA9-271PCCON_ST26.xml and is 85,330 bytes in size.

### FIELD OF INVENTION

This disclosure generally relates to high throughput systems and methods for identifying high producing clones useful for the production of therapeutic and diagnostic proteins.

### BACKGROUND OF INVENTION

Commercial production of a therapeutic protein requires a stable, high expressing recombinant cell line. Prior art methods have commonly relied on dihydrofolate reductase deficient (DHFR) Chinese Hamster Ovary (CHO) cell lines to manufacture clonal cell lines that produce the protein(s). High producing clones are obtained after repeated selection-based amplification of transfected pools of cells using agents such as methotrexate (MTX). This process is time-consuming and labor intensive and does not specifically identify clones that will produce proteins at high levels.

Flow cytometry, including fluorescence-activated cell sorting (FACS), has been used to improve the development and selection of high production CHO cell lines. This technology has enabled rapid identification and isolation of high production clones from a heterogeneous population of transfected cells, thereby decreasing the labor and time associated with random cloning methods. It can identify desired cells without the need for repeated MTX amplification of pools.

Once single-cell clones have been isolated via a FACS-based sorting method, however, screening an adequate number of clones to isolate a stable, high producing cell line still remains a time-consuming process. Therefore, a highly efficient and accurate screening method is extremely advantageous at this stage of cell line development. In addition, implementing a screen at the 96-well plate stage of clone development, for example, is desirable as it focuses further expansion on only those clones with high productivity. Analysis of cell culture media harvests is commonly used to identify clones secreting high levels of a therapeutic protein. However, this method is not optimal as it does not account for differences in either cell density or media volume between wells. As such, it may not accurately predict the clones with high specific productivity and high titer. Also, effort must often be spent to develop and optimize a new assay for each new therapeutic protein of interest.

Proteins of commercial interest are frequently composed of two or more polypeptide chains that are covalently and/or non-covalently associated. IgG antibodies, which are heterodimeric tetramers, and derivatives thereof are examples of such proteins. Production of multichain proteins typically involves co-expression of individual polypeptide chains and isolation of whole proteins.

WO 2008/036255 discloses a FACS- and reporter protein-based system for high throughput development of therapeutic proteins. to identify, select and produce a clonal population of recombinant eukaryotic host cells that stably and highly express a polypeptide of interest.

WO 2017/062722 discloses a method of fluorescence-activated cell sorting (FACS) to batch select producer cells expressing a target polypeptide.

A need still exists in the art for compositions and methods to screen clonal populations to select recombinant cell lines that stably produce high levels of multichain proteins of interest. This invention satisfies this need and provides related advantages as well.

### SUMMARY OF INVENTION

Methods and compositions useful to identify, select, and produce recombinant mammalian host cells that stably express a multimeric polypeptide of interest (“target polypeptide”) at high levels suitable for development and large-scale production of the target polypeptide are disclosed herein. The methods and compositions will find use in, for example, development and large-scale production of engineered antibodies, including crossover dual-variable domain (CODV) Ig-like proteins. Steinmetz A. et al. (2016) MAbs 8(5): 867-878.

An aspect of the present disclosure is a method for expressing a plurality of target polypeptides at a high level, the method comprising:
(a) culturing a plurality of mammalian host cells, each comprising a plurality of recombinant polynucleotides, wherein
each recombinant polynucleotide comprises a promoter, a nucleotide sequence encoding a unique cell surface marker polypeptide, and a nucleotide sequence encoding a unique target polypeptide, wherein both the nucleotide sequence encoding the unique cell surface marker polypeptide and the nucleotide sequence encoding the unique target polypeptide are transcribed on the same mRNA,
wherein the culturing is under conditions that permit expression of each unique cell surface marker polypeptide on the surface of the mammalian host cells and expression of each unique target polypeptide;
(b) contacting the cultured mammalian host cells of step (a) with a plurality of detectable agents, each uniquely capable of binding one or more of the unique cell surface marker polypeptides expressed on the surface of the mammalian host cells;
(c) performing at least one round of fluorescence-activated cell sorting on the contacted cells from step (b), thereby selecting one or more mammalian host cells that are uniquely bound by at least one of the plurality of detectable agents;
(d) preparing one or more clonal populations of the mammalian host cells selected in step (c);
(e) analyzing one or more clonal populations from step (d) by detecting level of expression of at least two unique cell surface marker polypeptides on each of said clonal populations;
(f) selecting one or more clonal populations having high expression level of the at least two unique cell surface marker polypeptides; and
(g) culturing one or more clonal populations selected in step (f) under conditions that permit expression of the plurality of target polypeptides at a high level.

In certain embodiments, step (c) comprises performing a single round of fluorescence-activated cell sorting on the contacted cells from step (b) with at least a first detectable agent and a second detectable agent, thereby selecting one or more mammalian host cells that are uniquely bound by at least both the first detectable agent and the second detectable agent.

In certain embodiments, step (c) comprises
(c1) performing a first round of fluorescence-activated cell sorting on at least a first cell surface marker polypeptide, thereby selecting one or more mammalian host cells that are bound by at least a first detectable agent; and
(c2) performing a second round of fluorescence-activated cell sorting, on cells selected in step (c1), on at least a second cell surface marker polypeptide, thereby selecting one or more mammalian host cells that are bound by at least both the first detectable agent and the second detectable agent.

In certain embodiments, step (e) is performed 7-28 days after step (d).

In certain embodiments, the analyzing in step (e) comprises flow cytometry.

In certain embodiments, the expression level of at least one of the plurality of unique cell surface marker polypeptides in step (f) is higher than a corresponding expression level of at least 70% of the clonal populations analyzed in step (e).

In certain embodiments, the expression level of each of the plurality of unique cell surface marker polypeptides in step (f) is higher than a corresponding expression level of at least 70% of the clonal populations analyzed in step (e).

In certain embodiments, the expression level of at least a first of the plurality of unique cell surface polypeptides in step (f) is higher than the expression level of at least a second of the plurality of unique cell surface polypeptides in step (f).

In certain embodiments, the expression level of a first of the plurality of unique cell surface polypeptides in step (f) is higher than the expression level of a second of the plurality of unique cell surface polypeptides in step (f).

In certain embodiments, the method further comprises:
(h) isolating at least a first unique target polypeptide and a second unique target polypeptide expressed in step (g) from the one or more selected clonal populations or from cell culture medium in which the one or more selected clonal populations are cultured.

In certain embodiments, the plurality of unique target polypeptides comprises 2 to 8 unique target polypeptides.

In certain embodiments, the plurality of unique target polypeptides comprises 2 to 4 unique target polypeptides.

In certain embodiments, the plurality of unique target polypeptides comprises 2 unique target polypeptides.

In certain embodiments, the plurality of unique target polypeptides comprises 3 unique target polypeptides.

In certain embodiments, the plurality of unique target polypeptides comprises 4 unique target polypeptides.

In certain embodiments, each recombinant polynucleotide comprises, from 5′ to 3′, the promoter, the nucleotide sequence encoding the unique cell surface marker polypeptide, and the nucleotide sequence encoding the unique target polypeptide.

In certain embodiments, each recombinant polynucleotide comprises 1 to 4 promoters, 1 to 4 nucleotide sequences each encoding a unique cell surface marker polypeptide, and 1 to 4 nucleotide sequences each encoding a unique target polypeptide, wherein one of the nucleotide sequences encoding a unique cell surface marker polypeptide is transcribed on the same mRNA as one of the nucleotide sequences encoding a unique target polypeptide.

In certain embodiments, each recombinant polynucleotide is a bicistronic polynucleotide comprising two promoters and two nucleotide sequences each encoding a unique target polypeptide, and further comprising one or two nucleotide sequences each encoding a unique cell surface marker polypeptide.

In certain embodiments, each bicistronic polynucleotide comprises a single nucleotide sequence encoding a unique cell surface marker polypeptide.

In certain embodiments, each bicistronic polynucleotide comprises two nucleotide sequences each encoding a unique cell surface marker polypeptide, and wherein each one of the nucleotide sequences encoding a unique cell surface marker polypeptide is transcribed on the same mRNA as one of the nucleotide sequences encoding a unique target polypeptide.

In certain embodiments, each recombinant polynucleotide comprises, from 5′ to 3′, one of the from 1-4 promoters, one of the from 1-4 nucleotide sequences encoding a unique cell surface marker polypeptide, and one of the from 1-4 nucleotide sequences encoding a unique target polypeptide. In certain embodiments, each recombinant polynucleotide comprises one promoter, one nucleotide sequence encoding a unique cell surface marker polypeptide, and one nucleotide sequence encoding a unique target polypeptide, wherein the nucleotide sequence encoding a unique cell surface marker polypeptide is transcribed on the same mRNA as the nucleotide sequence encoding a unique target polypeptide.

In certain embodiments, each recombinant polynucleotide further comprises an internal ribosome entry site (IRES) located 3′ to the nucleotide sequence encoding the unique cell surface marker polypeptide and 5′ to the nucleotide sequence encoding the unique target polypeptide.

In certain embodiments, each recombinant polynucleotide further comprises an alternate (non-ATG) start codon for translation initiation of the nucleotide sequence encoding the unique cell surface marker polypeptide and an ATG start codon for translation initiation of the nucleotide sequence encoding the unique target polypeptide.

In certain embodiments, each alternate (non-ATG) start codon is selected independently from the group consisting of CTG, GTG, TTG, ATT, ATA, and ACG.

In certain embodiments, each alternate (non-ATG) start codon is selected independently from the group consisting of GTG and TTG.

In certain embodiments, the nucleotide sequence encoding the unique cell surface marker polypeptide is devoid of any ATG triplets.

In certain embodiments, at least a first recombinant polynucleotide comprises, from 5′ to 3′, a first promoter, a nucleotide sequence encoding a first unique cell surface marker polypeptide, and a nucleotide sequence encoding a first unique target polypeptide; and at least a second recombinant polynucleotide comprises, from 5′ to 3′, a second promoter, a nucleotide sequence encoding a second unique target polypeptide, and a nucleotide sequence encoding a second unique cell surface marker polypeptide.

In certain embodiments, the first recombinant polynucleotide further comprises an internal ribosome entry site (IRES) located 3′ to the nucleotide sequence encoding the first unique cell surface marker polypeptide and 5′ to the nucleotide sequence encoding the first unique target polypeptide.

In certain embodiments, the first recombinant polynucleotide further comprises an alternate (non-ATG) start codon for translation initiation of the nucleotide sequence encoding the first unique cell surface marker polypeptide and an ATG start codon for translation initiation of the nucleotide sequence encoding the first unique target polypeptide.

In certain embodiments, the alternate (non-ATG) start codon is selected from the group consisting of CTG, GTG, TTG, ATT, ATA, and ACG.

In certain embodiments, the alternate (non-ATG) start codon is selected from the group consisting of GTG and TTG.

In certain embodiments, the nucleotide sequence encoding the first unique cell surface marker polypeptide is devoid of any ATG triplets.

In certain embodiments, the second recombinant polynucleotide further comprises an internal ribosome entry site (IRES) located 3′ to the nucleotide sequence encoding the second unique target polypeptide and 5′ to the nucleotide sequence encoding the second unique cell surface marker polypeptide.

In certain embodiments, each recombinant polynucleotide comprises, from 5′ to 3′, the promoter, the nucleotide sequence encoding the unique target polypeptide, and the nucleotide sequence encoding the unique cell surface marker polypeptide.

In certain embodiments, each recombinant polynucleotide further comprises an internal ribosome entry site (IRES) located 3′ to the nucleotide sequence encoding the unique target polypeptide and 5′ to the nucleotide sequence encoding the unique cell surface marker polypeptide.

In certain embodiments, at least one promoter is a β-actin promoter.

In certain embodiments, each promoter is a β-actin promoter.

In certain embodiments, at least one promoter is a hamster β-actin promoter.

In certain embodiments, each promoter is a hamster β-actin promoter.

In certain embodiments, at least a first unique cell surface marker polypeptide is selected from the group consisting of CD20, CD52, CD59, and variants thereof.

In certain embodiments, at least a second unique cell surface marker polypeptide is selected from the group consisting of CD20, CD52, CD59, and variants thereof.

In certain embodiments, each unique cell surface marker polypeptide is selected from the group consisting of CD20, CD52, CD59, and variants thereof.

In certain embodiments, at least a first unique cell surface marker polypeptide is selected from the group consisting of human CD52 and variants thereof.

In certain embodiments, at least a second unique cell surface marker polypeptide is selected from the group consisting of human CD52 and variants thereof.

In certain embodiments, each unique cell surface marker polypeptide is selected from the group consisting of human CD52 and variants thereof.

In certain embodiments, at least a first unique cell surface marker polypeptide is selected from the group consisting of CD20, CD52, and CD59.

In certain embodiments, at least a second unique cell surface marker polypeptide is selected from the group consisting of CD20, CD52, and CD59.

In certain embodiments, each unique cell surface marker polypeptide is selected from the group consisting of CD20, CD52, and CD59.

In certain embodiments, at least a first unique cell surface marker polypeptide is human CD52.

In certain embodiments, at least a second unique cell surface marker polypeptide is human CD52.

In certain embodiments, at least one unique cell surface marker polypeptide is a variant human CD52 comprising the amino acid sequence set forth in SEQ ID NO: 19 (GX₂NDTSQTSSPS) wherein X₂ is selected from the group consisting of A, G, I, L, and V. In certain embodiments, X₂ is A.

In certain embodiments, at least one unique cell surface marker polypeptide is a variant human CD52 comprising the amino acid sequence set forth in SEQ ID NO: 20 (GQNX₄TSQTSSPS) wherein X₄ is selected from the group consisting of A, G, I, L, and V. In certain embodiments, X₄ is A.

In certain embodiments, at least one unique cell surface marker polypeptide is a variant human CD52 comprising the amino acid sequence set forth in SEQ ID NO: 21 (GQNDTSX₇TSSPS) wherein X₇ is selected from the group consisting of A, G, I, L, and V. In certain embodiments, X₇ is A.

In certain embodiments, at least one unique cell surface marker polypeptide is a variant human CD52 comprising the amino acid sequence set forth in SEQ ID NO: 22 (GQNDTSQX₈SSPS) wherein X₈ is selected from the group consisting of A, G, I, L, and V. In certain embodiments, X₈ is A.

In certain embodiments, at least one unique cell surface marker polypeptide is a variant human CD52 comprising the amino acid sequence set forth in SEQ ID NO: 23 (GQNDTSQX₈X₉SPS), wherein each of X₈ and X₉ is independently selected from the group consisting of A, G, I, L, and V. In certain embodiments, each of X₈ and X₉ is A.

In certain embodiments, at least one unique cell surface marker polypeptide is a variant human CD52 comprising the amino acid sequence set forth in SEQ ID NO: 24 (GQNDTSQX₈SX₁₀PS), wherein each of X₈ and X₁₀ is independently selected from the group consisting of A, G, I, L, and V. In certain embodiments, each of X₈ and X₁₀ is A.

In certain embodiments, at least one unique cell surface marker polypeptide is a variant human CD52 comprising the amino acid sequence set forth in SEQ ID NO: 25 (GQNDTSQX₈X₉X₁₀PS), wherein each of X₈, X₉, and X₁₀ is independently selected from the group consisting of A, G, I, L, and V. In certain embodiments, each of X₈, X₉, and X₁₀ is A.

In certain embodiments, at least one unique cell surface marker polypeptide is a variant human CD52 comprising the amino acid sequence set forth in SEQ ID NO: 26 (GX₂NDTSQX₈X₉SPS), wherein each of X₂, X₈, and X₉ is independently selected from the group consisting of A, G, I, L, and V. In certain embodiments, each of X₂, X₈, and X₉ is A.

In certain embodiments, at least one unique cell surface marker polypeptide is a variant human CD52 comprising the amino acid sequence set forth in SEQ ID NO: 27 (GX₂NDTSQX₈SX₁₀PS), wherein each of X₂, X₈, and X₁₀ is independently selected from the group consisting of A, G, I, L, and V. In certain embodiments, each of X₂, X₈, and X₁₀ is A.

In certain embodiments, at least one unique cell surface marker polypeptide is a variant human CD52 comprising the amino acid sequence set forth in SEQ ID NO: 28 (GQNDTSX₇X₈X₉SPS), wherein each of X₇, X₈, and X₉ is independently selected from the group consisting of A, G, I, L, and V. In certain embodiments, each of X₇, X₈, and X₉ is A.

In certain embodiments, at least one unique cell surface marker polypeptide is a variant human CD52 comprising the amino acid sequence set forth in SEQ ID NO: 29 (GQNDTSX₇X₈SX₁₀PS), wherein each of X₇, X₈, and X₁₀ is independently selected from the group consisting of A, G, I, L, and V. In certain embodiments, each of X₇, X₈, and X₁₀ is A.

In certain embodiments, at least a first unique cell surface marker polypeptide comprises a variant human CD52 comprising the amino acid sequence set forth in SEQ ID NO: 19 (GX₂NDTSQTSSPS) wherein X₂ is selected from the group consisting of A, G, I, L, and V; and at least a second unique cell surface marker polypeptide comprises a variant human CD52 comprising an amino acid sequence selected from the group consisting of SEQ ID NO: 20 (GQNX₄TSQTSSPS) and SEQ ID NO: 21 (GQNDTSX₇TSSPS) wherein each of X₄ and X₇ is independently selected from the group consisting of A, G, I, L, and V.

In certain embodiments, at least a first unique cell surface marker polypeptide consists of a variant human CD52 consisting of the amino acid sequence set forth in SEQ ID NO: 19 (GX₂NDTSQTSSPS) wherein X₂ is selected from the group consisting of A, G, I, L, and V; and at least a second unique cell surface marker polypeptide consists of a variant human CD52 consisting of an amino acid sequence selected from the group consisting of SEQ ID NO: 20 (GQNX₄TSQTSSPS) and SEQ ID NO: 21 (GQNDTSX₇TSSPS) wherein each of X₄ and X₇ is independently selected from the group consisting of A, G, I, L, and V.

In certain embodiments, at least a first unique cell surface marker polypeptide comprises a variant human CD52 comprising the amino acid sequence set forth in SEQ ID NO: 2 (GANDTSQTSSPS); and at least a second unique cell surface marker polypeptide comprises a variant human CD52 comprising an amino acid sequence selected from the group consisting of SEQ ID NO: 4 (GQNATSQTSSPS) and SEQ ID NO: 7 (GQNDTSATSSPS).

In certain embodiments, at least a first unique cell surface marker polypeptide consists of a variant human CD52 consisting of the amino acid sequence set forth in SEQ ID NO: 2 (GANDTSQTSSPS); and at least a second unique cell surface marker polypeptide consists of a variant human CD52 consisting of an amino acid sequence selected from the group consisting of SEQ ID NO: 4 (GQNATSQTSSPS) and SEQ ID NO: 7 (GQNDTSATSSPS).

In certain embodiments, at least a first unique cell surface marker polypeptide comprises a variant human CD52 comprising the amino acid sequence set forth as SEQ ID NO: 2 (GANDTSQTSSPS); and at least a second unique cell surface marker polypeptide comprises a variant human CD52 comprising the amino acid sequence set forth as SEQ ID NO: 4 (GQNATSQTSSPS).

In certain embodiments, at least a first unique cell surface marker polypeptide consists of a variant human CD52 consisting of the amino acid sequence set forth as SEQ ID NO: 2 (GANDTSQTSSPS); and at least a second unique cell surface marker polypeptide consists of a variant human CD52 consisting of the amino acid sequence set forth as SEQ ID NO: 4 (GQNATSQTSSPS).

In certain embodiments, at least a first unique cell surface marker polypeptide comprises a variant human CD52 comprising the amino acid sequence set forth as SEQ ID NO: 2 (GANDTSQTSSPS); and at least a second unique cell surface marker polypeptide comprises a variant human CD52 comprising the amino acid sequence set forth as SEQ ID NO: 7 (GQNDTSATSSPS).

In certain embodiments, at least a first unique cell surface marker polypeptide consists of a variant human CD52 consisting of the amino acid sequence set forth as SEQ ID NO: 2 (GANDTSQTSSPS); and at least a second unique cell surface marker polypeptide consists of a variant human CD52 consisting of the amino acid sequence set forth as SEQ ID NO: 7 (GQNDTSATSSPS).

In certain embodiments, at least a first unique cell surface marker polypeptide comprises a variant human CD52 comprising the amino acid sequence set forth as SEQ ID NO: 4 (GQNATSQTSSPS); and at least a second unique cell surface marker polypeptide comprises a variant human CD52 comprising the amino acid sequence set forth as SEQ ID NO: 7 (GQNDTSATSSPS).

In certain embodiments, at least a first unique cell surface marker polypeptide comprises a variant human CD52 comprising the amino acid sequence set forth as SEQ ID NO: 20 (GQNX₄TSQTSSPS); at least a second unique cell surface marker polypeptide comprises a variant human CD52 comprising the amino acid sequence set forth as SEQ ID NO: 23 (GX₂NDTSQX₈X₉SPS); and at least a third unique cell surface marker polypeptide comprises a variant human CD52 comprising the amino acid sequence set forth as SEQ ID NO: 28 (GQNDTSX₇X₈X₉SPS), wherein each of X₄, X₇, X₈, and X₉ is independently selected from the group consisting of A, G, I, L, and V.

In certain embodiments, at least a first unique cell surface marker polypeptide comprises a variant human CD52 comprising the amino acid sequence set forth as SEQ ID NO: 20 (GQNX₄TSQTSSPS); at least a second unique cell surface marker polypeptide comprises a variant human CD52 comprising the amino acid sequence set forth as SEQ ID NO: 27 (GX₂NDTSQX₈SX₁₀PS); and at least a third unique cell surface marker polypeptide comprises a variant human CD52 comprising the amino acid sequence set forth as SEQ ID NO: 29 (GQNDTSX₇X₈SX₁₀PS) wherein each of X₄, X₇, X₈, and X₁₀ is independently selected from the group consisting of A, G, I, L, and V.

In certain embodiments, at least a first unique cell surface marker polypeptide comprises a variant human CD52 comprising the amino acid sequence set forth as SEQ ID NO: 4 (GQNATSQTSSPS); at least a second unique cell surface marker polypeptide comprises a variant human CD52 comprising the amino acid sequence set forth as SEQ ID NO: 15 (GANDTSQAASPS); and at least a third unique cell surface marker polypeptide comprises a variant human CD52 comprising the amino acid sequence set forth as SEQ ID NO: 17 (GQNDTSAAASPS).

In certain embodiments, at least a first unique cell surface marker polypeptide comprises a variant human CD52 comprising the amino acid sequence set forth as SEQ ID NO: 4 (GQNATSQTSSPS); at least a second unique cell surface marker polypeptide comprises a variant human CD52 comprising the amino acid sequence set forth as SEQ ID NO: 16 (GANDTSQASAPS); and at least a third unique cell surface marker polypeptide comprises a variant human CD52 comprising the amino acid sequence set forth as SEQ ID NO: 18 (GQNDTSAASAPS).

In certain embodiments, at least one unique target polypeptide comprises a therapeutic polypeptide or a therapeutic protein.

In certain embodiments, at least one unique target polypeptide is a polypeptide of a multi-chain protein.

In certain embodiments, each unique target polypeptide is a polypeptide of a multi-chain protein.

In certain embodiments, at least one unique target polypeptide is a polypeptide of an antibody.

In certain embodiments, each unique target polypeptide is a polypeptide of an antibody.

In certain embodiments, at least one unique target polypeptide is a polypeptide of a crossover dual-variable domain (CODV) Ig-like protein.

In certain embodiments, each unique target polypeptide is a polypeptide of a crossover dual-variable domain (CODV) Ig-like protein.

In certain embodiments, at least one unique target polypeptide is a polypeptide of a CODV triabody.

In certain embodiments, each unique target polypeptide is a polypeptide of a CODV triabody.

In certain embodiments, the recombinant mammalian host cell is a CHO cell.

A further aspect of the present disclosure is an engineered mammalian host cell comprising a plurality of recombinant polynucleotides, wherein each recombinant polynucleotide comprises a promoter, a nucleotide sequence encoding a unique cell surface marker polypeptide, and a nucleotide sequence encoding a unique target polypeptide, wherein both the nucleotide sequence encoding the unique cell surface marker polypeptide and the nucleotide sequence encoding the unique target polypeptide are transcribed on the same mRNA.

In certain embodiments, the plurality of unique target polypeptides comprises 2 to 8 unique target polypeptides.

In certain embodiments, the plurality of unique target polypeptides comprises 2 to 4 unique target polypeptides.

In certain embodiments, the plurality of unique target polypeptides comprises 2 unique target polypeptides.

In certain embodiments, the plurality of unique target polypeptides comprises 3 unique target polypeptides.

In certain embodiments, the plurality of unique target polypeptides comprises 4 unique target polypeptides.

In certain embodiments, each recombinant polynucleotide comprises, from 5′ to 3′, the promoter, the nucleotide sequence encoding the unique cell surface marker polypeptide, and the nucleotide sequence encoding the unique target polypeptide.

In certain embodiments, each recombinant polynucleotide comprises 1 to 4 promoters, 1 to 4 nucleotide sequences each encoding a unique cell surface marker polypeptide, and 1 to 4 nucleotide sequences each encoding a unique target polypeptide, wherein one of the nucleotide sequences encoding a unique cell surface marker polypeptide is transcribed on the same mRNA as one of the nucleotide sequences encoding a unique target polypeptide.

In certain embodiments, each recombinant polynucleotide is a bicistronic polynucleotide comprising two promoters and two nucleotide sequences each encoding a unique target polypeptide, and further comprising one or two nucleotide sequences each encoding a unique cell surface marker polypeptide.

In certain embodiments, each bicistronic polynucleotide comprises a single nucleotide sequence encoding a unique cell surface marker polypeptide.

In certain embodiments, each bicistronic polynucleotide comprises two nucleotide sequences each encoding a unique cell surface marker polypeptide, and wherein each one of the nucleotide sequences encoding a unique cell surface marker polypeptide is transcribed on the same mRNA as one of the nucleotide sequences encoding a unique target polypeptide.

In certain embodiments, each recombinant polynucleotide comprises, from 5′ to 3′, one of the from 1-4 promoters, one of the from 1-4 nucleotide sequences encoding a unique cell surface marker polypeptide, and one of the from 1-4 nucleotide sequences encoding a unique target polypeptide. In certain embodiments, each recombinant polynucleotide comprises one promoter, one nucleotide sequence encoding a unique cell surface marker polypeptide, and one nucleotide sequence encoding a unique target polypeptide, wherein the nucleotide sequence encoding a unique cell surface marker polypeptide is transcribed on the same mRNA as the nucleotide sequence encoding a unique target polypeptide.

In certain embodiments, each recombinant polynucleotide further comprises an internal ribosome entry site (IRES) located 3′ to the nucleotide sequence encoding the unique cell surface marker polypeptide and 5′ to the nucleotide sequence encoding the unique target polypeptide.

In certain embodiments, each recombinant polynucleotide further comprises an alternate (non-ATG) start codon for translation initiation of the nucleotide sequence encoding the unique cell surface marker polypeptide and an ATG start codon for translation initiation of the nucleotide sequence encoding the unique target polypeptide.

In certain embodiments, each alternate (non-ATG) start codon is selected independently from the group consisting of CTG, GTG, TTG, ATT, ATA, and ACG.

In certain embodiments, each alternate (non-ATG) start codon is selected independently from the group consisting of GTG and TTG.

In certain embodiments, the nucleotide sequence encoding the unique cell surface marker polypeptide is devoid of any ATG triplets.

In certain embodiments, at least a first recombinant polynucleotide comprises, from 5′ to 3′, a first promoter, a nucleotide sequence encoding a first unique cell surface marker polypeptide, and a nucleotide sequence encoding a first unique target polypeptide; and at least a second recombinant polynucleotide comprises, from 5′ to 3′, a second promoter, a nucleotide sequence encoding a second unique target polypeptide, and a nucleotide sequence encoding a second unique cell surface marker polypeptide.

In certain embodiments, the first recombinant polynucleotide further comprises an internal ribosome entry site (IRES) located 3′ to the nucleotide sequence encoding the first unique cell surface marker polypeptide and 5′ to the nucleotide sequence encoding the first unique target polypeptide.

In certain embodiments, the first recombinant polynucleotide further comprises an alternate (non-ATG) start codon for translation initiation of the nucleotide sequence encoding the first unique cell surface marker polypeptide and an ATG start codon for translation initiation of the nucleotide sequence encoding the first unique target polypeptide.

In certain embodiments, the alternate (non-ATG) start codon is selected from the group consisting of CTG, GTG, TTG, ATT, ATA, and ACG.

In certain embodiments, the alternate (non-ATG) start codon is selected from the group consisting of GTG and TTG.

In certain embodiments, the nucleotide sequence encoding the first unique cell surface marker polypeptide is devoid of any ATG triplets.

In certain embodiments, the second recombinant polynucleotide further comprises an internal ribosome entry site (IRES) located 3′ to the nucleotide sequence encoding the second unique target polypeptide and 5′ to the nucleotide sequence encoding the second unique cell surface marker polypeptide.

In certain embodiments, each recombinant polynucleotide comprises, from 5′ to 3′, the promoter, the nucleotide sequence encoding the unique target polypeptide, and the nucleotide sequence encoding the unique cell surface marker polypeptide.

In certain embodiments, each recombinant polynucleotide further comprises an internal ribosome entry site (IRES) located 3′ to the nucleotide sequence encoding the unique target polypeptide and 5′ to the nucleotide sequence encoding the unique cell surface marker polypeptide.

In certain embodiments, at least one promoter is a β-actin promoter.

In certain embodiments, each promoter is a β-actin promoter.

In certain embodiments, at least one promoter is a hamster β-actin promoter.

In certain embodiments, each promoter is a hamster β-actin promoter.

In certain embodiments, at least a first unique cell surface marker polypeptide is selected from the group consisting of CD20, CD52, CD59, and variants thereof.

In certain embodiments, at least a second unique cell surface marker polypeptide is selected from the group consisting of CD20, CD52, CD59, and variants thereof.

In certain embodiments, each unique cell surface marker polypeptide is selected from the group consisting of CD20, CD52, CD59, and variants thereof.

In certain embodiments, at least a first unique cell surface marker polypeptide is selected from the group consisting of human CD52 and variants thereof.

In certain embodiments, at least a second unique cell surface marker polypeptide is selected from the group consisting of human CD52 and variants thereof.

In certain embodiments, each unique cell surface marker polypeptide is selected from the group consisting of human CD52 and variants thereof.

In certain embodiments, at least a first unique cell surface marker polypeptide is selected from the group consisting of CD20, CD52, and CD59.

In certain embodiments, at least a second unique cell surface marker polypeptide is selected from the group consisting of CD20, CD52, and CD59.

In certain embodiments, each unique cell surface marker polypeptide is selected from the group consisting of CD20, CD52, and CD59.

In certain embodiments, at least a first unique cell surface marker polypeptide is human CD52.

In certain embodiments, at least a second unique cell surface marker polypeptide is human CD52.

In certain embodiments, at least one unique cell surface marker polypeptide is a variant human CD52 comprising the amino acid sequence set forth in SEQ ID NO: 19 (GX₂NDTSQTSSPS) wherein X₂ is selected from the group consisting of A, G, I, L, and V. In certain embodiments, X₂ is A.

In certain embodiments, at least one unique cell surface marker polypeptide is a variant human CD52 comprising the amino acid sequence set forth in SEQ ID NO: 20 (GQNX₄TSQTSSPS) wherein X₄ is selected from the group consisting of A, G, I, L, and V. In certain embodiments, X₄ is A.

In certain embodiments, at least one unique cell surface marker polypeptide is a variant human CD52 comprising the amino acid sequence set forth in SEQ ID NO: 21 (GQNDTSX₇TSSPS) wherein X₇ is selected from the group consisting of A, G, I, L, and V. In certain embodiments, X₇ is A.

In certain embodiments, at least one unique cell surface marker polypeptide is a variant human CD52 comprising the amino acid sequence set forth in SEQ ID NO: 22 (GQNDTSQX₈SSPS) wherein X₈ is selected from the group consisting of A, G, I, L, and V. In certain embodiments, X₈ is A.

In certain embodiments, at least one unique cell surface marker polypeptide is a variant human CD52 comprising the amino acid sequence set forth in SEQ ID NO: 23 (GQNDTSQX₈X₉SPS), wherein each of X₈ and X₉ is independently selected from the group consisting of A, G, I, L, and V. In certain embodiments, each of X₈ and X₉ is A.

In certain embodiments, at least one unique cell surface marker polypeptide is a variant human CD52 comprising the amino acid sequence set forth in SEQ ID NO: 24 (GQNDTSQX₈SX₁₀PS), wherein each of X₈ and X₁₀ is independently selected from the group consisting of A, G, I, L, and V. In certain embodiments, each of X₈ and X₁₀ is A.

In certain embodiments, at least one unique cell surface marker polypeptide is a variant human CD52 comprising the amino acid sequence set forth in SEQ ID NO: 25 (GQNDTSQX₈X₉X₁₀PS), wherein each of X₈, X₉, and X₁₀ is independently selected from the group consisting of A, G, I, L, and V. In certain embodiments, each of X₈, X₉, and X₁₀ is A.

In certain embodiments, at least one unique cell surface marker polypeptide is a variant human CD52 comprising the amino acid sequence set forth in SEQ ID NO: 26 (GX₂NDTSQX₈X₉SPS), wherein each of X₂, X₈, and X₉ is independently selected from the group consisting of A, G, I, L, and V. In certain embodiments, each of X₂, X₈, and X₉ is A.

In certain embodiments, at least one unique cell surface marker polypeptide is a variant human CD52 comprising the amino acid sequence set forth in SEQ ID NO: 27 (GX₂NDTSQX₈SX₁₀PS), wherein each of X₂, X₈, and X₁₀ is independently selected from the group consisting of A, G, I, L, and V. In certain embodiments, each of X₂, X₈, and X₁₀ is A.

In certain embodiments, at least one unique cell surface marker polypeptide is a variant human CD52 comprising the amino acid sequence set forth in SEQ ID NO: 28 (GQNDTSX₇X₈X₉SPS), wherein each of X₇, X₈, and X₉ is independently selected from the group consisting of A, G, I, L, and V. In certain embodiments, each of X₇, X₈, and X₉ is A.

In certain embodiments, at least one unique cell surface marker polypeptide is a variant human CD52 comprising the amino acid sequence set forth in SEQ ID NO: 29 (GQNDTSX₇X₈SX₁₀PS), wherein each of X₇, X₈, and X₁₀ is independently selected from the group consisting of A, G, I, L, and V. In certain embodiments, each of X₇, X₈, and X₁₀ is A.

In certain embodiments, at least a first unique cell surface marker polypeptide comprises a variant human CD52 comprising the amino acid sequence set forth in SEQ ID NO: 19 (GX₂NDTSQTSSPS) wherein X₂ is selected from the group consisting of A, G, I, L, and V; and at least a second unique cell surface marker polypeptide comprises a variant human CD52 comprising an amino acid sequence selected from the group consisting of SEQ ID NO: 20 (GQNX₄TSQTSSPS) and SEQ ID NO: 21 (GQNDTSX₇TSSPS) wherein each of X₄ and X₇ is independently selected from the group consisting of A, G, I, L, and V.

In certain embodiments, at least a first unique cell surface marker polypeptide consists of a variant human CD52 consisting of the amino acid sequence set forth in SEQ ID NO: 19 (GX₂NDTSQTSSPS) wherein X₂ is selected from the group consisting of A, G, I, L, and V; and at least a second unique cell surface marker polypeptide consists of a variant human CD52 consisting of an amino acid sequence selected from the group consisting of SEQ ID NO: 20 (GQNX₄TSQTSSPS) and SEQ ID NO: 21 (GQNDTSX₇TSSPS) wherein each of X₄ and X₇ is independently selected from the group consisting of A, G, I, L, and V.

In certain embodiments, at least a first unique cell surface marker polypeptide comprises a variant human CD52 comprising the amino acid sequence set forth in SEQ ID NO: 2 (GANDTSQTSSPS); and at least a second unique cell surface marker polypeptide comprises a variant human CD52 comprising an amino acid sequence selected from the group consisting of SEQ ID NO: 4 (GQNATSQTSSPS) and SEQ ID NO: 7 (GQNDTSATSSPS).

In certain embodiments, at least a first unique cell surface marker polypeptide consists of a variant human CD52 consisting of the amino acid sequence set forth in SEQ ID NO: 2 (GANDTSQTSSPS); and at least a second unique cell surface marker polypeptide consists of a variant human CD52 consisting of an amino acid sequence selected from the group consisting of SEQ ID NO: 4 (GQNATSQTSSPS) and SEQ ID NO: 7 (GQNDTSATSSPS).

In certain embodiments, at least a first unique cell surface marker polypeptide comprises a variant human CD52 comprising the amino acid sequence set forth as SEQ ID NO: 2 (GANDTSQTSSPS); and at least a second unique cell surface marker polypeptide comprises a variant human CD52 comprising the amino acid sequence set forth as SEQ ID NO: 4 (GQNATSQTSSPS).

In certain embodiments, at least a first unique cell surface marker polypeptide consists of a variant human CD52 consisting of the amino acid sequence set forth as SEQ ID NO: 2 (GANDTSQTSSPS); and at least a second unique cell surface marker polypeptide consists of a variant human CD52 consisting of the amino acid sequence set forth as SEQ ID NO: 4 (GQNATSQTSSPS).

In certain embodiments, at least a first unique cell surface marker polypeptide comprises a variant human CD52 comprising the amino acid sequence set forth as SEQ ID NO: 2 (GANDTSQTSSPS); and at least a second unique cell surface marker polypeptide comprises a variant human CD52 comprising the amino acid sequence set forth as SEQ ID NO: 7 (GQNDTSATSSPS).

In certain embodiments, at least a first unique cell surface marker polypeptide consists of a variant human CD52 consisting of the amino acid sequence set forth as SEQ ID NO: 2 (GANDTSQTSSPS); and at least a second unique cell surface marker polypeptide consists of a variant human CD52 consisting of the amino acid sequence set forth as SEQ ID NO: 7 (GQNDTSATSSPS).

In certain embodiments, at least a first unique cell surface marker polypeptide comprises a variant human CD52 comprising the amino acid sequence set forth as SEQ ID NO: 4 (GQNATSQTSSPS); and at least a second unique cell surface marker polypeptide comprises a variant human CD52 comprising the amino acid sequence set forth as SEQ ID NO: 7 (GQNDTSATSSPS).

In certain embodiments, at least a first unique cell surface marker polypeptide comprises a variant human CD52 comprising the amino acid sequence set forth as SEQ ID NO: 20 (GQNX₄TSQTSSPS); at least a second unique cell surface marker polypeptide comprises a variant human CD52 comprising the amino acid sequence set forth as SEQ ID NO: 23 (GX₂NDTSQX₈X₉SPS); and at least a third unique cell surface marker polypeptide comprises a variant human CD52 comprising the amino acid sequence set forth as SEQ ID NO: 28 (GQNDTSX₇X₈X₉SPS), wherein each of X₄, X₇, X₈, and X₉ is independently selected from the group consisting of A, G, I, L, and V.

In certain embodiments, at least a first unique cell surface marker polypeptide comprises a variant human CD52 comprising the amino acid sequence set forth as SEQ ID NO: 20 (GQNX₄TSQTSSPS); at least a second unique cell surface marker polypeptide comprises a variant human CD52 comprising the amino acid sequence set forth as SEQ ID NO: 27 (GX₂NDTSQX₈SX₁₀PS); and at least a third unique cell surface marker polypeptide comprises a variant human CD52 comprising the amino acid sequence set forth as SEQ ID NO: 29 (GQNDTSX₇X₈SX₁₀PS) wherein each of X₄, X₇, X₈, and X₁₀ is independently selected from the group consisting of A, G, I, L, and V.

In certain embodiments, at least a first unique cell surface marker polypeptide comprises a variant human CD52 comprising the amino acid sequence set forth as SEQ ID NO: 4 (GQNATSQTSSPS); at least a second unique cell surface marker polypeptide comprises a variant human CD52 comprising the amino acid sequence set forth as SEQ ID NO: 15 (GANDTSQAASPS); and at least a third unique cell surface marker polypeptide comprises a variant human CD52 comprising the amino acid sequence set forth as SEQ ID NO: 17 (GQNDTSAAASPS).

In certain embodiments, at least a first unique cell surface marker polypeptide comprises a variant human CD52 comprising the amino acid sequence set forth as SEQ ID NO: 4 (GQNATSQTSSPS); at least a second unique cell surface marker polypeptide comprises a variant human CD52 comprising the amino acid sequence set forth as SEQ ID NO: 16 (GANDTSQASAPS); and at least a third unique cell surface marker polypeptide comprises a variant human CD52 comprising the amino acid sequence set forth as SEQ ID NO: 18 (GQNDTSAASAPS).

In certain embodiments, at least one unique target polypeptide comprises a therapeutic polypeptide or a therapeutic protein.

In certain embodiments, at least one unique target polypeptide is a polypeptide of a multi-chain protein.

In certain embodiments, each unique target polypeptide is a polypeptide of a multi-chain protein.

In certain embodiments, at least one unique target polypeptide is a polypeptide of an antibody.

In certain embodiments, each unique target polypeptide is a polypeptide of an antibody.

In certain embodiments, at least one unique target polypeptide is a polypeptide of a crossover dual-variable domain (CODV) Ig-like protein.

In certain embodiments, each unique target polypeptide is a polypeptide of a crossover dual-variable domain (CODV) Ig-like protein.

In certain embodiments, at least one unique target polypeptide is a polypeptide of a CODV triabody.

In certain embodiments, each unique target polypeptide is a polypeptide of a CODV triabody.

In certain embodiments, the recombinant mammalian host cell is a CHO cell.

A further aspect of the present disclosure is an isolated variant human CD52 comprising an amino acid sequence consisting of SEQ ID NO: 19 (GX₂NDTSQTSSPS) wherein X₂ is selected from the group consisting of A, G, I, L, and V. In certain embodiments, X₂ is A.

A further aspect of the present disclosure is an isolated variant human CD52 comprising an amino acid sequence consisting of SEQ ID NO: 20 (GQNX₄TSQTSSPS) wherein X₄ is selected from the group consisting of A, G, I, L, and V. In certain embodiments, X₄ is A.

Yet a further aspect of the present disclosure is an isolated variant human CD52 comprising an amino acid sequence consisting of SEQ ID NO: 22 (GQNDTSQX₈SSPS) wherein X₈ is selected from the group consisting of A, G, I, L, and V. In certain embodiments, X₈ is A.

### BRIEF DESCRIPTION OF THE DRAWINGS

FIG. 1 is a schematic depiction of a recombinant polynucleotide and corresponding mRNA transcript in accordance with the instant disclosure. In this depiction, only a single representative recombinant polynucleotide is shown. The nucleotide sequence encoding a unique cell surface marker polypeptide (reporter) has a TTG start codon, and the nucleotide sequence encoding a unique target polypeptide (therapeutic protein) has an ATG start codon.

FIG. 2A is a graph depicting binding of mAbs 4B10 and 7F11 to CHO cells expressing wild-type hCD52 (WT) and each of 10 single point mutants of hCD52, each substituting alanine (A) for the indicated amino acid of WT. Also shown is binding to non-transfected CHO cells (CHO parental).

FIG. 2B is a graph depicting binding of mAbs CF1D12, 5F7, 3G7, 4G7, 9D9, and 11C11 to CHO cells expressing wild-type hCD52 (WT) and each of 10 single point mutants of hCD52, each substituting alanine (A) for the indicated amino acid of WT. Also shown is binding to non-transfected CHO cells (CHO parental).

FIG. 2C is a graph depicting binding of mAbs Campath-1H, 2C3, 12G6, and 23E6 to CHO cells expressing wild-type hCD52 (WT) and each of 10 single point mutants of hCD52, each substituting alanine (A) for the indicated amino acid of WT. Also shown is binding to non-transfected CHO cells (CHO parental).

FIG. 3 depicts a sequence alignment of native (wild-type) hCD52 (SEQ ID NO: 30) and engineered mutant hCD52 (Mut4; SEQ ID NO: 31) including a non-AUG (alternate) start codon with optimal Kozak context, mutation of all internal ATG codons, eliminated splice sites, and a single point mutation substituting alanine for aspartic acid at amino acid 4 of hCD52. Mut4 is bound by Campath-1H (C-1H) but not by mAb 9D9. Partial amino acid sequence shown corresponds to SEQ ID NO: 4.

FIG. 4 depicts a sequence alignment of native (wild-type) hCD52 (SEQ ID NO: 30) and engineered mutant hCD52 (Mut8; SEQ ID NO: 32) including a non-AUG (alternate) start codon with optimal Kozak context, mutation of all internal ATG codons, eliminated splice sites, and a single point mutation substituting alanine for threonine at amino acid 8 of hCD52. Mut8 is bound by mAb 9D9 but not by Campath-1H (C-1H). Partial amino acid sequence shown corresponds to SEQ ID NO: 8.

FIG. 5 depicts a sequence alignment of native (wild-type) hCD52 (SEQ ID NO: 30) and engineered mutant hCD52 (Mut8/10; SEQ ID NO: 33) including a non-AUG (alternate) start codon with optimal Kozak context, mutation of all internal ATG codons, eliminated splice sites, and two point mutations substituting alanine for threonine at amino acid 8 of hCD52 and alanine for serine at amino acid 10 of hCD52. Mut8/10 is bound by mAb 9D9 but not by Campath-1H (C-1H). Partial amino acid sequence shown corresponds to SEQ ID NO: 13.

FIG. 6 is four flow cytometry histograms depicting binding by Campath-1H or 9D9 to the indicated mutants of hCD52, where the polynucleotide encoding each mutant of hCD52 has an ATG start codon.

FIG. 7 is four flow cytometry histograms depicting binding by Campath-1H or 9D9 to the indicated mutants of hCD52, where the polynucleotide encoding each mutant of hCD52 has an ATG start codon.

FIG. 8 is four flow cytometry histograms depicting binding by Campath-1H or 9D9 to the indicated mutants of hCD52, where the polynucleotide encoding each mutant of hCD52 has the indicated start codon (ATG, CTG, GTG, or TTG).

FIG. 9 is a pair of bar graphs depicting relative binding of Mut4 (left graph) and Mut8 (right graph) by Campath-1H or 9D9, where each hCD52 mutant is encoded by a polynucleotide with the indicated start codon (ATG, CTG, GTG, or TTG).

FIG. 10 is a flow cytometry histogram depicting the relative levels of reporter expression and gene of interest (GOI) expression depending on choice of start codon (ATG/AUG, CTG/CUG, GTG, or TTG) for the reporter. The depicted partial sequences correspond to SEQ ID NOs: 35 to 38.

FIG. 11 is a collection of bar graphs depicting a scheme for a three-reporter system using the indicated hCD52 mutants (Mut4, Mut2/8/9, and Mut7/8/9) and the indicated antibodies (7F11, 9D9, and Campath-1H).

FIG. 12 is a collection of bar graphs depicting a scheme for a three-reporter system using the indicated hCD52 mutants (Mut4, Mut2/8/10, and Mut7/8/10) and the indicated antibodies (7F11, 9D9, and Campath-1H).

FIG. 13A is a schematic depicting four vectors encoding a CODV triabody. There are two reporters in this system.

FIG. 13B is a schematic depicting two bi-cistronic vectors encoding a CODV triabody. There are two reporters in this system.

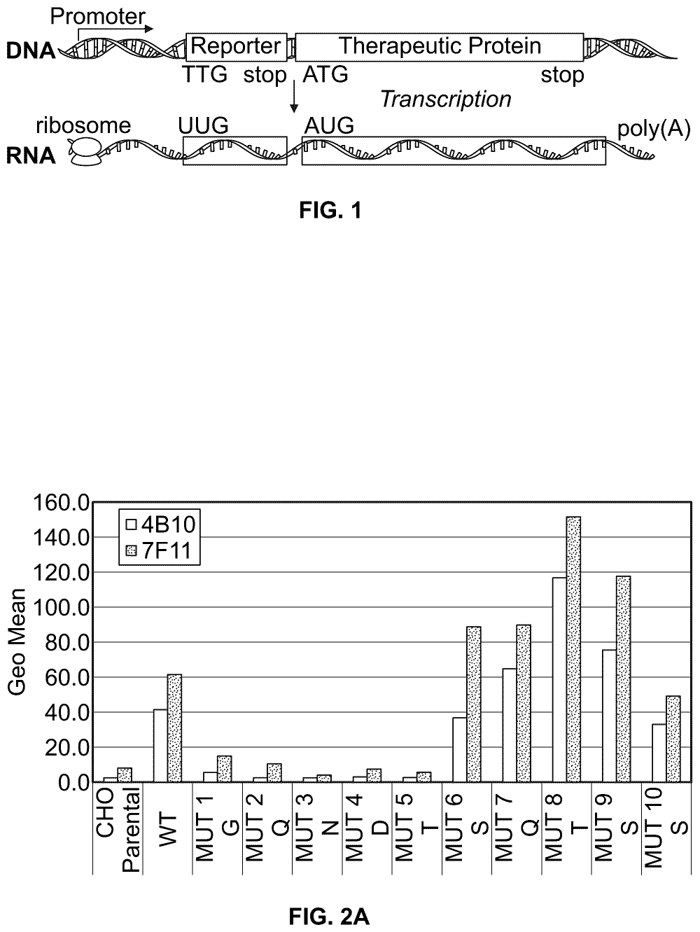

### DETAILED DESCRIPTION OF INVENTION

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

As used herein, certain terms have the following defined meanings.

### I. Definitions

The practice of the present invention will employ, unless otherwise indicated, conventional techniques of immunology, molecular biology, microbiology, cell biology and recombinant DNA. which are within the skill of the art. See, e.g., Sambrook, Fritsch and Maniatis, Molecular Cloning: A Laboratory Manual, 2^{nd} edition (1989); Current Protocols in Molecular Biology (F. M. Ausubel, et al. eds., (1987)); the series Methods in Enzymology (Academic Press, Inc.): PCR 2: A Practical Approach (M. J. MacPherson. B. D. Hames and G. R. Taylor eds. (1995)), Harlow and Lane, eds. (1988) Antibodies, A Laboratory Manual, and Animal Cell Culture (R. I. Freshney, ed. (1987)).

As used in the specification and claims, the singular form “a,” “an,” and “the” include plural references unless the context clearly dictates otherwise. For example, the term “a cell” includes a plurality of cells, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

As used herein, the term “polynucleotide” or “nucleotide sequence” intends a polymeric form of nucleotides of any length, examples of which include, but are not limited to a gene or gene fragment, exons, introns, messenger RNA (mRNA), transfer RNA, ribosomal RNA, ribozymes, complementary DNA (cDNA), recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs.

As used herein, the term “polypeptide” refers to a polymeric form of amino acids or amino acid analogs of any length, examples of which include full-length single-chain polypeptides, full-length single-chain proteins, fragments of full-length single-chain polypeptides, fragments of full-length single-chain proteins; full-length multi-chain polypeptides, full-length multi-chain proteins, fragments of full-length multi-chain polypeptides, fragments of full-length multi-chain proteins; and full-length single chains of multi-chain polypeptides, full-length single chains of multi-chain proteins, fragments of single chains of full-length multi-chain polypeptides, and fragments of single chains of full-length multi-chain proteins.

As used herein, a “multi-chain polypeptide” or “multi-chain protein” refers to any polypeptide or protein comprised of two or more single-chain polypeptides. The two or more single-chain polypeptides can be associated by any combination of covalent bonds, e.g., disulfide bonds, and/or non-covalent bonds, e.g., ionic or hydrogen bonds. For example, a conventional IgG antibody is comprised of two heavy chains linked by one or more disulfide bonds in the hinge region, and two light chains, each light chain linked to a single heavy chain by one or more disulfide bonds between their respective variable light (VL) and variable heavy (VH) domains. As another example, a CODV triabody is comprised of two different heavy chains (CODV heavy chain and Fab heavy chain, with reciprocal knobs and holes) and two different light chains (CODV light chain and Fab light chain).

Additional examples of multi-chain proteins, also referred to as multimeric proteins, include homomeric and heteromeric multimeric proteins, including, without limitation, certain enzymes, ion channels, receptors, cell adhesion molecules, voltage-gated potassium channels, and therapeutic proteins (e.g., insulin and α-galactosidase). Many soluble and membrane proteins form homomultimeric complexes in a cell, and a majority of proteins in the Protein Data Bank are homomultimeric. Approximately 65% of proteins in prokaryotes and 55% of those in eukaryotes exist as dimers or higher-order complexes (not including very high-order structures such as the cytoskeleton, ribosomes, etc.). Among complexes, homomers are about 4× more frequent than heteromers in unicellular species, whereas the two types are equally frequent in vertebrates. As a consequence, heteromers constitute about 10% of proteins in unicellular species but nearly 30% in vertebrates. Lynch M, Mol Biol Evol. 29(5): 1353-1366 (2012). Homooligomers are responsible for the diversity and specificity of many pathways, and they may mediate and regulate gene expression, activity of enzymes, ion channels, receptors, and cell adhesion processes. The voltage-gated potassium channels in the plasma membrane of a neuron are heteromultimeric proteins composed of four of forty known alpha subunits.

As used herein, the term “IRES” or “a polynucleotide having the biological activity of IRES” is intended to include any molecule such as a polynucleotide or its reverse transcript which is able to initiate translation of the polynucleotide operatively linked to the IRES without the benefit of a cap site in a eukaryotic cell. An IRES or a polynucleotide having the biological activity of IRES can be identical to sequences found in nature, such as the picornavirus IRES, or they can be non-naturally occurring or non-native sequences that perform the same function when introduced into a suitable host cell.

As used herein, the term “alternate start codon” is intended to include any non-ATG polynucleotide (typically a triplet) that functions as a start site for translation initiation with reduced efficiency relative to that of an ATG start codon. Naturally occurring alternate start codon usage is known in the art and described for example in Kozak (1991) J Cell Biol. 115(4): 887-903; Mehdi et al. (1990) Gene 91: 173-178; Kozak (1989)Mol Cell Biol. 9(11): 5073-5080. In general, alternate start codons have decreased translation efficiencies compared to that of an ATG; for example, the alternate start codon GTG may have 3-5% translation efficiency compared to that of an ATG (100%). The translation efficiency of an alternate start codon can also be affected by its sequence context: for example, an optimal Kozak consensus sequence is reported to have a positive effect on translation initiation at alternate start codons. Mehdi et al. (1990) Gene 91:173-178; Kozak (1989) Mol Cell Biol. 9(11): 5073-5080. The complete Kozak consensus sequence is GCCRCCATGG (SEQ ID NO: 34). where the start codon ATG is in bold font, the A of the ATG start codon is designated as the +1 position, and “R” at position −3 is a purine (A or G). The two most highly conserved positions are a purine, preferably an A, at −3 and a G at +4 (Kozak (1991) J Cell Biol. 115(4): 887-903). Alternate start codon usage is described for attenuated expression of a selectable marker in U.S. Patent Publication 2006/0172382 and U.S. Patent Publication 2006/0141577. One of skill in the art will recognize that the sequences described herein as DNA will have correlative sequences as RNA molecules, e.g., DNA sequence ATG, for example, would correspond to RNA sequence AUG.

As used herein, a “high expression level” refers to a level of expression that is higher than the expression level of at least 50, 70, 80, 90, 95 or 99% of the total cells analyzed.

As used herein, “CD20” refers to a four transmembrane protein that is expressed on the surface of B cells, starting at the pre-B cell stage and also on mature B cells in the bone marrow and in the periphery. CD20 presents three surface-available antigenic regions. cDNA and amino acid sequences for human and mouse CD20 are available from GenBank, e.g., accession nos. NM_152866, NM 021950, and NM_152867 (human); NM_007641 (mouse); NP_068769, NP_690605, and NP_690606 (human); and NP_031667 (mouse). Full-length human CD20 is 297 amino acids long, and full-length mouse CD20 is 291 amino acids long.

CD52, also known as Campath-1H antigen, refers to a short glycoprotein expressed on mature lymphocytes; while its function is not well known, it is thought to inhibit cell adhesion. cDNA and amino acid sequences for human and mouse CD52 are available from GenBank, e.g., accession nos. BC000644 (human); NM_013706 (mouse); AAH00644 (human); and EDL30035 (mouse). Full-length human CD52 is 61 amino acids long, including a 42-amino acid signal sequence, and full-length mouse CD52 is 74 amino acids long, including a 23-amino acid signal sequence.

CD59, also known as membrane attack complex (MAC)-inhibitory protein and protectin, is a cell surface glycoprotein that inhibits complement component C9 from polymerizing and forming the membrane attack complex. cDNA and amino acid sequences for human and mouse CD59 are available from GenBank, e.g., accession nos. NM_203331, NM_0006111, NM_001127223, NM_001127225, and NM_001127226 (human); NM_181858 and NM_001368215 (mouse); NP_000602, NP_001120695, NP_001120697, NP_001120698, and NP_001120699 (human); and NP_862906 and NP_001355144 (mouse). Full-length human CD59 is 128 amino acids long, including a 25-amino acid signal sequence, and full-length mouse CD59 is 129 amino acids long, including a 23-amino acid signal sequence.

As used herein, a “therapeutic polypeptide” or “therapeutic protein” refers to any protein or polypeptide that can be produced in host cells and in the aspects exemplified herein, which is selected because of its potential as a therapeutic agent or drug, e.g., an antibody, an antibody fragment, an antibody-like molecule (e.g., a crossover dual-variable domain (CODV) Ig-like protein), or enzyme.

### II. Methods

An aspect of the present disclosure is a method for expressing a plurality of target polypeptides at a high level, the method comprising:
(a) culturing a plurality of mammalian host cells, each comprising a plurality of recombinant polynucleotides, wherein
each recombinant polynucleotide comprises a promoter, a nucleotide sequence encoding a unique cell surface marker polypeptide, and a nucleotide sequence encoding a unique target polypeptide, wherein both the nucleotide sequence encoding the unique cell surface marker polypeptide and the nucleotide sequence encoding the unique target polypeptide are transcribed on the same mRNA,
wherein the culturing is under conditions that permit expression of each unique cell surface marker polypeptide on the surface of the mammalian host cells and expression of each unique target polypeptide;
(b) contacting the cultured mammalian host cells of step (a) with a plurality of detectable agents, each uniquely capable of binding one or more of the unique cell surface marker polypeptides expressed on the surface of the mammalian host cells;
(c) performing at least one round of fluorescence-activated cell sorting on the contacted cells from step (b), thereby selecting one or more mammalian host cells that are uniquely bound by at least one of the plurality of detectable agents;
(d) preparing one or more clonal populations of the mammalian host cells selected in step (c);
(e) analyzing one or more clonal populations from step (d) by detecting level of expression of at least two unique cell surface marker polypeptides on each of said clonal populations;
(f) selecting one or more clonal populations having high expression level of the at least two unique cell surface marker polypeptides; and
(g) culturing one or more clonal populations selected in step (f) under conditions that permit expression of the plurality of target polypeptides at a high level.

The plurality of mammalian host cells can be a population mammalian host cells, for example a population of cells of a cell line of mammalian cells. The plurality or population of cells typically includes, at step (a), 100 or more, 500 or more, 1,000 or more, 10,000 or more, 50,000 or more, 100,000 or more, 500,000 or more, or 10⁶ or more cells. The cells typically are all of one type, for example, CHO cells, HEK-293 cells, BHK-21 cells, HepG2 cells, BAE-1 cells, SH-SY5Y cells, myeloma cells, hybridoma cells, HeLa cells, Vero cells, NIH3T3 cells, WEHI231 cells, YAC cells, Jurkat cells, and derivatives thereof, for example CHO-K1 cells and CHO/DHFR⁻ cells.

The culturing under conditions that permit expression of each unique cell surface marker polypeptide on the surface of the mammalian host cells and expression of each unique target polypeptide can include conditions such as are typical for mammalian cells in tissue culture, e.g., 37° C. in humidified air supplemented with 5% CO₂ in a suitable medium such as DMEM, Ham's F12, or serum-free media, optionally supplemented with fetal calf serum (FCS) or fetal bovine serum (FBS), L-glutamine, and penicillin/streptomycin. The culturing can take place in any suitable format, for example, multiwell plate culture, dish culture, bulk culture, or bioreactor culture. The culturing can permit time for expression of the cell surface marker polypeptides, as well as time for cell population expansion.

The plurality of recombinant polynucleotides includes two or more different recombinant polynucleotides. In certain embodiments, the plurality of recombinant polynucleotides includes 2, 3, 4, 5, 6, 7, 8, 9, 10, 11, or 12 or more different recombinant polynucleotides. In certain embodiments, the plurality of recombinant polynucleotides includes 2 different recombinant polynucleotides. In certain embodiments, the plurality of recombinant polynucleotides includes 3 different recombinant polynucleotides. In certain embodiments, the plurality of recombinant polynucleotides includes 4 different recombinant polynucleotides. In certain embodiments, the plurality of recombinant polynucleotides includes 5 different recombinant polynucleotides. In certain embodiments, the plurality of recombinant polynucleotides includes 6 different recombinant polynucleotides. In certain embodiments, the plurality of recombinant polynucleotides includes 7 different recombinant polynucleotides. In certain embodiments, the plurality of recombinant polynucleotides includes 8 different recombinant polynucleotides.

Of course, each mammalian host cell can contain more than a single copy of each recombinant polynucleotide. For example, each mammalian host cell can contain about 10¹ to about 10⁶ copies of each recombinant polynucleotide.

Additionally, each mammalian host cell can contain about the same number or, alternatively, different numbers of copies of each recombinant polynucleotide. In certain embodiments, it is preferred that each mammalian host cell contains about the same number of copies of at least two different recombinant polynucleotides. In certain embodiments, it is preferred that each mammalian host cell contains about the same number of copies of each recombinant polynucleotide. In certain other embodiments, it is preferred that each mammalian host cell contains a different number of copies of at least two different recombinant polynucleotides. In certain other embodiments, it is preferred that each mammalian host cell contains a different number of copies of each recombinant polynucleotide. The number of copies of each different recombinant polynucleotide can be varied and selected based on conditions used to introduce the different recombinant polynucleotides into the host mammalian cells.

Each recombinant polynucleotide comprises a promoter, a nucleotide sequence encoding a unique cell surface marker polypeptide, and a nucleotide sequence encoding a unique target polypeptide, wherein both the nucleotide sequence encoding the unique cell surface marker polypeptide and the nucleotide sequence encoding the unique target polypeptide are transcribed on the same mRNA.

Because each recombinant polynucleotide comprises a nucleotide sequence encoding a unique cell surface marker polypeptide, and because both the nucleotide sequence encoding the unique cell surface marker polypeptide and the nucleotide sequence encoding the unique target polypeptide are transcribed on the same mRNA, each mammalian host cell can express as many unique, i.e., different, cell surface marker polypeptides as there are different recombinant polynucleotides. For example, a mammalian host cell transfected with two different recombinant polynucleotides can express two unique, i.e., different, cell surface marker polypeptides. Similarly, a mammalian host cell transfected with three different recombinant polynucleotides can express three unique, i.e., different, cell surface marker polypeptides, and so on. Whatever the number of nucleotide sequences encoding unique, i.e., different, cell surface marker polypeptides, expression of each such cell surface marker polypeptide will be associated with expression of a corresponding unique, i.e., different, target polypeptide.

Similarly, because each recombinant polynucleotide comprises a nucleotide sequence encoding a unique target polypeptide, and because both the nucleotide sequence encoding the unique cell surface marker polypeptide and the nucleotide sequence encoding the unique target polypeptide are transcribed on the same mRNA, each mammalian host cell can express as many unique, i.e., different, target polypeptides as there are different recombinant polynucleotides. For example, a mammalian host cell transfected with two different recombinant polynucleotides can express two unique, i.e., different, target polypeptides. Similarly, a mammalian host cell transfected with three different recombinant polynucleotides can express three unique, i.e., different, target polypeptides, and so on. Whatever the number of nucleotide sequences encoding unique, i.e., different, target polypeptides, expression of each such target polypeptide will be associated with expression of a corresponding unique, i.e., different, cell surface marker polypeptide.

Step (b) entails contacting the cultured mammalian host cells of step (a) with a plurality of detectable agents, each uniquely capable of binding one or more of the unique cell surface marker polypeptides expressed on the surface of the mammalian host cells. In certain embodiments, at least one unique detectable agent is capable of binding only one unique cell surface marker polypeptide. In certain embodiments, each unique detectable agent is capable of binding only one unique cell surface marker polypeptide. In certain embodiments, at least one unique detectable agent is capable of binding more than one unique cell surface marker polypeptide; for example, such unique detectable agent may be capable of binding two different, i.e., unique, cell surface marker polypeptides.

Step (c) entails performing at least one round of fluorescence-activated cell sorting (FACS) on the contacted cells from step (b), thereby selecting one or more mammalian host cells that are uniquely bound by at least one of the plurality of detectable agents. In certain embodiments, step (c) comprises performing a single round of FACS on the contacted cells from step (b) with at least a first detectable agent and a second detectable agent, thereby selecting one or more mammalian host cells that are uniquely bound by at least both the first detectable agent and the second detectable agent. In certain embodiments, step (c) comprises performing a single round of FACS on the contacted cells from step (b) with 3 or more detectable agents, thereby selecting one or more mammalian host cells that are uniquely bound by the 3 or more detectable agents. In certain embodiments, step (c) comprises performing a single round of FACS on the contacted cells from step (b) with 3 detectable agents, thereby selecting one or more mammalian host cells that are uniquely bound by the 3 detectable agents.

In certain embodiments, step (c) comprises performing a single round of FACS on the contacted cells from step (b) with 4 detectable agents, thereby selecting one or more mammalian host cells that are uniquely bound by the 4 detectable agents. In certain embodiments, step (c) comprises performing a single round of FACS on the contacted cells from step (b) with 5 detectable agents, thereby selecting one or more mammalian host cells that are uniquely bound by the 5 detectable agents. In certain embodiments, step (c) comprises performing a single round of FACS on the contacted cells from step (b) with 6 detectable agents, thereby selecting one or more mammalian host cells that are uniquely bound by the 6 detectable agents. In certain embodiments, step (c) comprises performing a single round of FACS on the contacted cells from step (b) with 7 detectable agents, thereby selecting one or more mammalian host cells that are uniquely bound by the 7 detectable agents. In certain embodiments, step (c) comprises performing a single round of FACS on the contacted cells from step (b) with 8 detectable agents, thereby selecting one or more mammalian host cells that are uniquely bound by the 8 detectable agents.

In certain embodiments, step (c) comprises
(c1) performing a first round of fluorescence-activated cell sorting on at least a first cell surface marker polypeptide, thereby selecting one or more mammalian host cells that are bound by at least a first detectable agent; and
(c2) performing a second round of fluorescence-activated cell sorting, on cells selected in step (c1), on at least a second cell surface marker polypeptide, thereby selecting one or more mammalian host cells that are bound by at least both the first detectable agent and the second detectable agent.

Of course, in certain embodiments, yet additional rounds of FACS are performed, thereby selecting one or more mammalian host cells that are bound by any number of specific detectable agents. For example, in certain embodiments step (c) further comprises
(c3) performing a third round of FACS, on cells selected in step (c2), on at least a third cell surface marker polypeptide, thereby selecting one or more mammalian host cells that are bound by at least the first, second, and third detectable agents.

Similarly, in certain embodiments step (c) further comprises
(c4) performing a fourth round of FACS, on cells selected in step (c3), on at least a fourth cell surface marker polypeptide, thereby selecting one or more mammalian host cells that are bound by at least the first, second, third, and fourth detectable agents.

In certain embodiments, step (e) is performed 7-28 days after step (d). In certain embodiments, step (e) is performed 7-21 days after step (d). In certain embodiments, step (e) is performed 7-14 days after step (d). In certain embodiments, step (e) is performed 7-13 days after step (d). In certain embodiments, step (e) is performed 7-12 days after step (d). In certain embodiments, step (e) is performed 7-11 days after step (d). In certain embodiments, step (e) is performed 7-10 days after step (d). In certain embodiments, step (e) is performed 7-9 days after step (d). In certain embodiments, step (e) is performed 7-8 days after step (d). In certain embodiments, step (e) is performed 7-9 days after step (d). In certain embodiments, step (e) is performed 7 days after step (d).

In certain embodiments, step (e) is performed 8-28 days after step (d). In certain embodiments, step (e) is performed 8-21 days after step (d). In certain embodiments, step (e) is performed 8-14 days after step (d). In certain embodiments, step (e) is performed 8-13 days after step (d). In certain embodiments, step (e) is performed 8-12 days after step (d). In certain embodiments, step (e) is performed 8-11 days after step (d). In certain embodiments, step (e) is performed 8-10 days after step (d). In certain embodiments, step (e) is performed 8-9 days after step (d). In certain embodiments, step (e) is performed 8 days after step (d).

In certain embodiments, step (e) is performed 9-28 days after step (d). In certain embodiments, step (e) is performed 9-21 days after step (d). In certain embodiments, step (e) is performed 9-14 days after step (d). In certain embodiments, step (e) is performed 9-13 days after step (d). In certain embodiments, step (e) is performed 9-12 days after step (d). In certain embodiments, step (e) is performed 9-11 days after step (d). In certain embodiments, step (e) is performed 9-10 days after step (d). In certain embodiments, step (e) is performed 9 days after step (d).

In certain embodiments, step (e) is performed 10-28 days after step (d). In certain embodiments, step (e) is performed 10-21 days after step (d). In certain embodiments, step (e) is performed 10-14 days after step (d). In certain embodiments, step (e) is performed 10-13 days after step (d). In certain embodiments, step (e) is performed 10-12 days after step (d). In certain embodiments, step (e) is performed 10-11 days after step (d). In certain embodiments, step (e) is performed 10 days after step (d).

In certain embodiments, step (e) is performed 11-28 days after step (d). In certain embodiments, step (e) is performed 11-21 days after step (d). In certain embodiments, step (e) is performed 11-14 days after step (d). In certain embodiments, step (e) is performed 11-13 days after step (d). In certain embodiments, step (e) is performed 11-12 days after step (d). In certain embodiments, step (e) is performed 11 days after step (d).

In certain embodiments, step (e) is performed 12-28 days after step (d). In certain embodiments, step (e) is performed 12-21 days after step (d). In certain embodiments, step (e) is performed 12-14 days after step (d). In certain embodiments, step (e) is performed 12-13 days after step (d). In certain embodiments, step (e) is performed 12 days after step (d).

In certain embodiments, step (e) is performed 13-28 days after step (d). In certain embodiments, step (e) is performed 13-21 days after step (d). In certain embodiments, step (e) is performed 13-14 days after step (d). In certain embodiments, step (e) is performed 13 days after step (d).

In certain embodiments, step (e) is performed 14-28 days after step (d). In certain embodiments, step (e) is performed 14-21 days after step (d). In certain embodiments, step (e) is performed 14 days after step (d).

In certain embodiments, step (e) is performed 15-28 days after step (d). In certain embodiments, step (e) is performed 15-21 days after step (d). In certain embodiments, step (e) is performed 15 days after step (d).

In certain embodiments, step (e) is performed 16-28 days after step (d). In certain embodiments, step (e) is performed 16-21 days after step (d). In certain embodiments, step (e) is performed 16 days after step (d).

In certain embodiments, step (e) is performed 17-28 days after step (d). In certain embodiments, step (e) is performed 17-21 days after step (d). In certain embodiments, step (e) is performed 17 days after step (d).

In certain embodiments, step (e) is performed 18-28 days after step (d). In certain embodiments, step (e) is performed 18-21 days after step (d). In certain embodiments, step (e) is performed 18 days after step (d).

In certain embodiments, step (e) is performed 19-28 days after step (d). In certain embodiments, step (e) is performed 19-21 days after step (d). In certain embodiments, step (e) is performed 19 days after step (d).

In certain embodiments, step (e) is performed 20-28 days after step (d). In certain embodiments, step (e) is performed 20-21 days after step (d). In certain embodiments, step (e) is performed 20 days after step (d).

In certain embodiments, step (e) is performed 21-28 days after step (d). In certain embodiments, step (e) is performed 21 days after step (d).

In certain embodiments, step (e) is performed 22-28 days after step (d). In certain embodiments, step (e) is performed 22 days after step (d).

In certain embodiments, step (e) is performed 23-28 days after step (d). In certain embodiments, step (e) is performed 23 days after step (d).

In certain embodiments, step (e) is performed 24-28 days after step (d). In certain embodiments, step (e) is performed 24 days after step (d).

In certain embodiments, step (e) is performed 25-28 days after step (d). In certain embodiments, step (e) is performed 25 days after step (d).

In certain embodiments, step (e) is performed 26-28 days after step (d). In certain embodiments, step (e) is performed 26 days after step (d).

In certain embodiments, step (e) is performed 27-28 days after step (d). In certain embodiments, step (e) is performed 27 days after step (d).

In certain embodiments, step (e) is performed 28 days after step (d).

In certain embodiments, the analyzing in step (e) comprises flow cytometry. In certain embodiments, the analyzing by flow cytometry in step (e) includes cell sorting. In certain other embodiments, the analyzing by flow cytometry in step (e) does not include cell sorting.

In certain embodiments, the expression level of at least one of the plurality of unique cell surface marker polypeptides in step (f) is higher than a corresponding expression level of at least 70% of the clonal populations analyzed in step (e). For example, if one of the plurality of unique cell surface marker polypeptides is CD52, then the expression level of CD52 in step (f) may be higher than a the CD52 expression level of at least 70% of the clonal populations analyzed in step (e). In certain embodiments, the at least one of the plurality of unique cell surface marker polypeptides in step (f) is one unique cell surface marker. In certain other embodiments, the at least one of the plurality of unique cell surface marker polypeptides in step (f) is two unique cell surface marker polypeptides. In certain other embodiments, the at least one of the plurality of unique cell surface marker polypeptides in step (f) is three unique cell surface marker polypeptides, four unique cell surface marker polypeptides, etc. The same principle applies mutatis mutandis for any number of unique cell surface marker polypeptides.

In certain embodiments, the expression level of each of the plurality of unique cell surface marker polypeptides in step (f) is higher than a corresponding expression level of at least 70% of the clonal populations analyzed in step (e).

In certain embodiments, the expression level of at least a first of the plurality of unique cell surface marker polypeptides in step (f) is higher than the expression level of at least a second of the plurality of unique cell surface marker polypeptides in step (f). For example, if one of the plurality of unique cell surface markers polypeptides is a first CD52 variant, and another of the plurality of unique cell surface marker polypeptides is a second CD52 variant, then the expression level of at least the first CD52 variant in step (f) may be higher than the expression level of at least the second CD52 variant in step (f). Similarly, if one of the plurality of unique cell surface marker polypeptides is a first CD52 variant, another of the plurality of unique cell surface marker polypeptides is a second CD52 variant, and yet another of the plurality of unique cell surface marker polypeptides is a third CD52 variant, then the expression level of at least the first CD52 variant in step (f) may be higher than the expression level of both the second CD52 variant and the third CD52 variant in step (f). As another example, if one of the plurality of unique cell surface marker polypeptides is a first CD52 variant, another of the plurality of unique cell surface marker polypeptides is a second CD52 variant, and yet another of the plurality of unique cell surface marker polypeptides is a third CD52 variant, then the expression level of at least the first CD52 variant and the expression level of the second CD52 variant in step (f) may be higher than the expression level of the third CD52 variant in step (f). The same principles apply mutatis mutandis for any number of unique cell surface marker polypeptides.

In certain embodiments, the expression level of a first of the plurality of unique cell surface polypeptides in step (f) is higher than the expression level of a second of the plurality of unique cell surface polypeptides in step (f).

In certain embodiments, the method further comprises:
(h) isolating at least a first unique target polypeptide and a second unique target polypeptide expressed in step (g) from the one or more selected clonal populations or from cell culture medium in which the one or more selected clonal populations are cultured. Any suitable method for isolating or purifying a desired expressed target polypeptide can be used. For example, in certain embodiments, the isolating can involve affinity chromatography, gel filtration/size exclusion, hydrophobic interaction chromatography, immunoprecipitation, ion exchange chromatography, or any combination thereof. For secreted target polypeptides, the isolating or purifying can be done directly from the cell media or from a dialysate and/or concentrate therefrom. For other target polypeptides, the isolating or purifying can include a cell lysis step or membrane disruption step. Such general isolation and purification methods are well-known to persons skilled in the art.

In certain embodiments, the plurality of unique target polypeptides comprises 2 to 8 unique target polypeptides.

In certain embodiments, the plurality of unique target polypeptides comprises 2 to 4 unique target polypeptides.

In certain embodiments, the plurality of unique target polypeptides comprises 2 unique target polypeptides.

In certain embodiments, the plurality of unique target polypeptides comprises 3 unique target polypeptides.

In certain embodiments, the plurality of unique target polypeptides comprises 4 unique target polypeptides.

In certain embodiments, the plurality of unique target polypeptides comprises 5 unique target polypeptides.

In certain embodiments, the plurality of unique target polypeptides comprises 6 unique target polypeptides.

In certain embodiments, the plurality of unique target polypeptides comprises 7 unique target polypeptides.

In certain embodiments, the plurality of unique target polypeptides comprises 8 unique target polypeptides.

In certain embodiments, the plurality of unique target polypeptides comprises 8 or more unique target polypeptides.

In certain embodiments, each recombinant polynucleotide comprises, from 5′ to 3′, the promoter, the nucleotide sequence encoding the unique cell surface marker polypeptide, and the nucleotide sequence encoding the unique target polypeptide.

In certain embodiments, each recombinant polynucleotide further comprises an internal ribosome entry site (IRES) located 3′ to the nucleotide sequence encoding the unique cell surface marker polypeptide and 5′ to the nucleotide sequence encoding the unique target polypeptide.

In certain embodiments, each recombinant polynucleotide further comprises an alternate (non-ATG) start codon for translation initiation of the nucleotide sequence encoding the unique cell surface marker polypeptide and an ATG start codon for translation initiation of the nucleotide sequence encoding the unique target polypeptide.

In certain embodiments, each alternate (non-ATG) start codon is selected independently from the group consisting of CTG, GTG, TTG, ATT, ATA, and ACG. That is, the alternate (non-ATG) start codons can be the same or different; some or all can be the same, or some or all can be different.

In certain embodiments, at least one alternate start codon is CTG.

In certain embodiments, at least one alternate start codon is GTG.

In certain embodiments, at least one alternate start codon is TTG.

In certain embodiments, at least one alternate start codon is ATT.

In certain embodiments, at least one alternate start codon is ATA.

In certain embodiments, at least one alternate start codon is ACG.

In certain embodiments, each alternate (non-ATG) start codon is selected independently from the group consisting of GTG and TTG. In certain embodiments, at least one alternate start codon is GTG. In certain embodiments, at least one alternate start codon is TTG. In certain embodiments, each alternate (non-ATG) start codon is GTG. In certain other embodiments, each alternate (non-ATG) start codon is TTG.

In certain embodiments, the nucleotide sequence encoding the unique cell surface marker polypeptide is devoid of any ATG triplets.

In certain embodiments, at least a first recombinant polynucleotide comprises, from 5′ to 3′, a first promoter, a nucleotide sequence encoding a first unique cell surface marker polypeptide, and a nucleotide sequence encoding a first unique target polypeptide; and at least a second recombinant polynucleotide comprises, from 5′ to 3′, a second promoter, a nucleotide sequence encoding a second unique target polypeptide, and a nucleotide sequence encoding a second unique cell surface marker polypeptide. As pointed out above, the instant disclosure also contemplates embodiments which further can include, without limitation, at least a third recombinant polynucleotide with corresponding elements, at least a fourth recombinant polynucleotide with corresponding elements, at least a fifth recombinant polynucleotide with corresponding elements, at least a sixth recombinant polynucleotide with corresponding elements, at least a seventh recombinant polynucleotide with corresponding elements, and at least an eighth recombinant polynucleotide with corresponding elements.

In certain embodiments, the first recombinant polynucleotide further comprises an internal ribosome entry site (IRES) located 3′ to the nucleotide sequence encoding the first unique cell surface marker polypeptide and 5′ to the nucleotide sequence encoding the first unique target polypeptide.

In certain embodiments, the first recombinant polynucleotide further comprises an alternate (non-ATG) start codon for translation initiation of the nucleotide sequence encoding the first unique cell surface marker polypeptide and an ATG start codon for translation initiation of the nucleotide sequence encoding the first unique target polypeptide.

In certain embodiments, the alternate (non-ATG) start codon is selected from the group consisting of CTG, GTG, TTG, ATT, ATA, and ACG.

In certain embodiments, the alternate (non-ATG) start codon is selected from the group consisting of GTG and TTG.

In certain embodiments, the alternate (non-ATG) start codon is CTG.

In certain embodiments, the alternate (non-ATG) start codon is GTG.

In certain embodiments, the alternate (non-ATG) start codon is TTG.

In certain embodiments, the alternate (non-ATG) start codon is ATT.

In certain embodiments, the alternate (non-ATG) start codon is ATA.

In certain embodiments, the alternate (non-ATG) start codon is ACG.

In certain embodiments, the nucleotide sequence encoding the first unique cell surface marker polypeptide is devoid of any ATG triplets.

In certain embodiments, the second recombinant polynucleotide further comprises an internal ribosome entry site (IRES) located 3′ to the nucleotide sequence encoding the second unique target polypeptide and 5′ to the nucleotide sequence encoding the second unique cell surface marker polypeptide.

In certain embodiments, each recombinant polynucleotide comprises, from 5′ to 3′, the promoter, the nucleotide sequence encoding the unique target polypeptide, and the nucleotide sequence encoding the unique cell surface marker polypeptide.

In certain embodiments, each recombinant polynucleotide further comprises an internal ribosome entry site (IRES) located 3′ to the nucleotide sequence encoding the unique target polypeptide and 5′ to the nucleotide sequence encoding the unique cell surface marker polypeptide.

Each of the promoters is selected independently of any other promoter. In certain embodiments, no two promoters are the same. In certain embodiments, at least two promoters are the same. In certain embodiments, all of the promoters are the same.

In certain embodiments, at least one promoter is a β-actin promoter.

In certain embodiments, each promoter is a β-actin promoter.

In certain embodiments, at least one promoter is a hamster β-actin promoter.

In certain embodiments, each promoter is a hamster β-actin promoter.

In certain embodiments, at least a first unique cell surface marker polypeptide is selected from the group consisting of CD20, CD52, CD59, and variants thereof.

As used herein, unless otherwise indicated, CD20, CD52, and CD59 encompass both human and non-human forms of CD20, CD52, and CD59. In certain embodiments, CD20 is human CD20 (hCD20). In certain embodiments, CD52 is human CD52 (hCD52). In certain embodiments, CD59 is human CD59 (hCD59). Non-human forms of CD20, CD52, and CD59 include, without limitation, mouse, rat, and non-human primate forms of CD20, CD52, and CD59.

As used herein, a “variant” refers to a mutated version of the specified molecule wherein at least one amino acid residue differs from the wild-type molecule. For example, a “variant” can include 1, 2, 3, 4, 5, 6, 7, 8, 9, 10, 11, or 12 point mutations compared to the wild-type polypeptide, provided that the variant is capable of being expressed on the surface of the host mammalian cell. For variants with two or more point mutations, said mutations can be adjacent or non-adjacent to one another. In some embodiments, at least two point mutations are adjacent to one another. In certain other embodiments, no two point mutations are adjacent to one another. In some embodiments, some point mutations are adjacent to one another, and other point mutations are non-adjacent to one another.

In certain embodiments, the first unique cell surface marker polypeptide is CD20 or a variant thereof.

In certain embodiments, the first unique cell surface marker polypeptide is CD20.

In certain embodiments, the first unique cell surface marker polypeptide is a variant CD20.

In certain embodiments, the first unique cell surface marker polypeptide is CD52 or a variant thereof.

In certain embodiments, the first unique cell surface marker polypeptide is CD52.

In certain embodiments, the first unique cell surface marker polypeptide is a variant CD52.

In certain embodiments, the first unique cell surface marker polypeptide is CD59 or a variant thereof.

In certain embodiments, the first unique cell surface marker polypeptide is CD59.

In certain embodiments, the first unique cell surface marker polypeptide is variant CD59.

In certain embodiments, at least a second unique cell surface marker polypeptide is selected from the group consisting of CD20, CD52, CD59, and variants thereof. In such embodiments, the first and second unique cell surface marker polypeptides are different from one another. For example, the first unique cell surface marker polypeptide can be wild-type CD52, and the second unique cell surface marker polypeptide can be a variant CD52. As another example, the first unique cell surface marker polypeptide can be a first variant CD52, and the second unique cell surface marker polypeptide can be a second variant CD52, wherein the first and second variant CD52 molecules are different.

In certain embodiments, each unique cell surface marker polypeptide is selected from the group consisting of CD20, CD52, CD59, and variants thereof.

In certain embodiments, at least a first unique cell surface marker polypeptide is selected from the group consisting of human CD52 and variants thereof.

In certain embodiments, at least a second unique cell surface marker polypeptide is selected from the group consisting of human CD52 and variants thereof. In such embodiments, the first and second unique cell surface marker polypeptides are different from one another. For example, the first unique cell surface marker polypeptide can be wild-type human CD52, and the second unique cell surface marker polypeptide can be a variant human CD52. As another example, the first unique cell surface marker polypeptide can be a first variant human CD52, and the second unique cell surface marker polypeptide can be a second variant human CD52, wherein the first and second variant human CD52 molecules are different.

In certain embodiments, each unique cell surface marker polypeptide is selected from the group consisting of human CD52 and variants thereof.

In certain embodiments, at least a first unique cell surface marker polypeptide is selected from the group consisting of CD20, CD52, and CD59.

In certain embodiments, at least a second unique cell surface marker polypeptide is selected from the group consisting of CD20, CD52, and CD59.

In certain embodiments, each unique cell surface marker polypeptide is selected from the group consisting of CD20, CD52, and CD59.

In certain embodiments, at least a first unique cell surface marker polypeptide is human CD52.

In certain embodiments, at least a second unique cell surface marker polypeptide is human CD52.

In certain embodiments, at least one unique cell surface marker polypeptide is a variant human CD52 comprising the amino acid sequence set forth in SEQ ID NO: 19 (GX₂NDTSQTSSPS) wherein X₂ is selected from the group consisting of A, G, I, L, and V.

In certain embodiments, X₂ is A.

In certain embodiments, at least one unique cell surface marker polypeptide is a variant human CD52 comprising the amino acid sequence set forth in SEQ ID NO: 20 (GQNX₄TSQTSSPS) wherein X₄ is selected from the group consisting of A, G, I, L, and V.

In certain embodiments, X₄ is A.

In certain embodiments, at least one unique cell surface marker polypeptide is a variant human CD52 comprising the amino acid sequence set forth in SEQ ID NO: 21 (GQNDTSX₇TSSPS) wherein X₇ is selected from the group consisting of A, G, I, L, and V.

In certain embodiments, X₇ is A.

In certain embodiments, at least one unique cell surface marker polypeptide is a variant human CD52 comprising the amino acid sequence set forth in SEQ ID NO: 22 (GQNDTSQX₈SSPS) wherein X₈ is selected from the group consisting of A, G, I, L, and V.

In certain embodiments, X₈ is A.

In certain embodiments, at least one unique cell surface marker polypeptide is a variant human CD52 comprising the amino acid sequence set forth in SEQ ID NO: 23 (GQNDTSQX₈X₉SPS), wherein each of X₈ and X₉ is independently selected from the group consisting of A, G, I, L, and V. In certain embodiments, each of X₈ and X₉ is A.

In certain embodiments, at least one unique cell surface marker polypeptide is a variant human CD52 comprising the amino acid sequence set forth in SEQ ID NO: 24 (GQNDTSQX₈SX₁₀PS), wherein each of X₈ and X₁₀ is independently selected from the group consisting of A, G, I, L, and V. In certain embodiments, each of X₈ and X₁₀ is A.

In certain embodiments, at least one unique cell surface marker polypeptide is a variant human CD52 comprising the amino acid sequence set forth in SEQ ID NO: 25 (GQNDTSQX₈X₉X₁₀PS), wherein each of X₈, X₉, and X₁₀ is independently selected from the group consisting of A, G, I, L, and V. In certain embodiments, each of X₈, X₉, and X₁₀ is A.

In certain embodiments, at least one unique cell surface marker polypeptide is a variant human CD52 comprising the amino acid sequence set forth in SEQ ID NO: 26 (GX₂NDTSQX₈X₉SPS), wherein each of X₂, X₈, and X₉ is independently selected from the group consisting of A, G, I, L, and V. In certain embodiments, each of X₂, X₈, and X₉ is A.

In certain embodiments, at least one unique cell surface marker polypeptide is a variant human CD52 comprising the amino acid sequence set forth in SEQ ID NO: 27 (GX₂NDTSQX₈SX₁₀PS), wherein each of X₂, X₈, and X₁₀ is independently selected from the group consisting of A, G, I, L, and V. In certain embodiments, each of X₂, X₈, and X₁₀ is A.

In certain embodiments, at least one unique cell surface marker polypeptide is a variant human CD52 comprising the amino acid sequence set forth in SEQ ID NO: 28 (GQNDTSX₇X₈X₉SPS), wherein each of X₇, X₈, and X₉ is independently selected from the group consisting of A, G, I, L, and V. In certain embodiments, each of X₇, X₈, and X₉ is A.

In certain embodiments, at least one unique cell surface marker polypeptide is a variant human CD52 comprising the amino acid sequence set forth in SEQ ID NO: 29 (GQNDTSX₇X₈SX₁₀PS), wherein each of X₇, X₈, and X₁₀ is independently selected from the group consisting of A, G, I, L, and V. In certain embodiments, each of X₇, X₈, and X₁₀ is A.

In certain embodiments, at least a first unique cell surface marker polypeptide comprises a variant human CD52 comprising the amino acid sequence set forth in SEQ ID NO: 19 (GX₂NDTSQTSSPS) wherein X₂ is selected from the group consisting of A, G, I, L, and V; and at least a second unique cell surface marker polypeptide comprises a variant human CD52 comprising an amino acid sequence selected from the group consisting of SEQ ID NO: 20 (GQNX₄TSQTSSPS) and SEQ ID NO: 21 (GQNDTSX₇TSSPS) wherein each of X₄ and X₇ is independently selected from the group consisting of A, G, I, L, and V.

In certain embodiments, at least a first unique cell surface marker polypeptide consists of a variant human CD52 consisting of the amino acid sequence set forth in SEQ ID NO: 19 (GX₂NDTSQTSSPS) wherein X₂ is selected from the group consisting of A, G, I, L, and V; and at least a second unique cell surface marker polypeptide consists of a variant human CD52 consisting of an amino acid sequence selected from the group consisting of SEQ ID NO: 20 (GQNX₄TSQTSSPS) and SEQ ID NO: 21 (GQNDTSX₇TSSPS) wherein each of X₄ and X₇ is independently selected from the group consisting of A, G, I, L, and V.

In certain embodiments, at least a first unique cell surface marker polypeptide comprises a variant human CD52 comprising the amino acid sequence set forth in SEQ ID NO: 2 (GANDTSQTSSPS); and at least a second unique cell surface marker polypeptide comprises a variant human CD52 comprising an amino acid sequence selected from the group consisting of SEQ ID NO: 4 (GQNATSQTSSPS) and SEQ ID NO: 7 (GQNDTSATSSPS).

In certain embodiments, at least a first unique cell surface marker polypeptide consists of a variant human CD52 consisting of the amino acid sequence set forth in SEQ ID NO: 2 (GANDTSQTSSPS); and at least a second unique cell surface marker polypeptide consists of a variant human CD52 consisting of an amino acid sequence selected from the group consisting of SEQ ID NO: 4 (GQNATSQTSSPS) and SEQ ID NO: 7 (GQNDTSATSSPS).

In certain embodiments, at least a first unique cell surface marker polypeptide comprises a variant human CD52 comprising the amino acid sequence set forth as SEQ ID NO: 2 (GANDTSQTSSPS); and at least a second unique cell surface marker polypeptide comprises a variant human CD52 comprising the amino acid sequence set forth as SEQ ID NO: 4 (GQNATSQTSSPS).

In certain embodiments, at least a first unique cell surface marker polypeptide consists of a variant human CD52 consisting of the amino acid sequence set forth as SEQ ID NO: 2 (GANDTSQTSSPS); and at least a second unique cell surface marker polypeptide consists of a variant human CD52 consisting of the amino acid sequence set forth as SEQ ID NO: 4 (GQNATSQTSSPS).

In certain embodiments, at least a first unique cell surface marker polypeptide comprises a variant human CD52 comprising the amino acid sequence set forth as SEQ ID NO: 2 (GANDTSQTSSPS); and at least a second unique cell surface marker polypeptide comprises a variant human CD52 comprising the amino acid sequence set forth as SEQ ID NO: 7 (GQNDTSATSSPS).

In certain embodiments, at least a first unique cell surface marker polypeptide consists of a variant human CD52 consisting of the amino acid sequence set forth as SEQ ID NO: 2 (GANDTSQTSSPS); and at least a second unique cell surface marker polypeptide consists of a variant human CD52 consisting of the amino acid sequence set forth as SEQ ID NO: 7 (GQNDTSATSSPS).

In certain embodiments, at least a first unique cell surface marker polypeptide comprises a variant human CD52 comprising the amino acid sequence set forth as SEQ ID NO: 4 (GQNATSQTSSPS); and at least a second unique cell surface marker polypeptide comprises a variant human CD52 comprising the amino acid sequence set forth as SEQ ID NO: 7 (GQNDTSATSSPS).

In certain embodiments, at least a first unique cell surface marker polypeptide comprises a variant human CD52 comprising the amino acid sequence set forth as SEQ ID NO: 20 (GQNX₄TSQTSSPS); at least a second unique cell surface marker polypeptide comprises a variant human CD52 comprising the amino acid sequence set forth as SEQ ID NO: 23 (GX₂NDTSQX₈X₉SPS); and at least a third unique cell surface marker polypeptide comprises a variant human CD52 comprising the amino acid sequence set forth as SEQ ID NO: 28 (GQNDTSX₇X₈X₉SPS), wherein each of X₄, X₇, X₈, and X₉ is independently selected from the group consisting of A, G, I, L, and V.

In certain embodiments, at least a first unique cell surface marker polypeptide comprises a variant human CD52 comprising the amino acid sequence set forth as SEQ ID NO: 20 (GQNX₄TSQTSSPS); at least a second unique cell surface marker polypeptide comprises a variant human CD52 comprising the amino acid sequence set forth as SEQ ID NO: 27 (GX₂NDTSQX₈SX₁₀PS); and at least a third unique cell surface marker polypeptide comprises a variant human CD52 comprising the amino acid sequence set forth as SEQ ID NO: 29 (GQNDTSX₇X₈SX₁₀PS) wherein each of X₄, X₇, X₈, and X₁₀ is independently selected from the group consisting of A, G, I, L, and V.

In certain embodiments, at least a first unique cell surface marker polypeptide comprises a variant human CD52 comprising the amino acid sequence set forth as SEQ ID NO: 4 (GQNATSQTSSPS); at least a second unique cell surface marker polypeptide comprises a variant human CD52 comprising the amino acid sequence set forth as SEQ ID NO: 15 (GANDTSQAASPS); and at least a third unique cell surface marker polypeptide comprises a variant human CD52 comprising the amino acid sequence set forth as SEQ ID NO: 17 (GQNDTSAAASPS).

In certain embodiments, at least a first unique cell surface marker polypeptide comprises a variant human CD52 comprising the amino acid sequence set forth as SEQ ID NO: 4 (GQNATSQTSSPS); at least a second unique cell surface marker polypeptide comprises a variant human CD52 comprising the amino acid sequence set forth as SEQ ID NO: 16 (GANDTSQASAPS); and at least a third unique cell surface marker polypeptide comprises a variant human CD52 comprising the amino acid sequence set forth as SEQ ID NO: 18 (GQNDTSAASAPS).

In certain embodiments, at least one unique target polypeptide comprises a therapeutic polypeptide or a therapeutic protein.

In certain embodiments, at least one unique target polypeptide is a polypeptide of a multi-chain protein.

In certain embodiments, each unique target polypeptide is a polypeptide of a multi-chain protein.

In certain embodiments, at least one unique target polypeptide is a polypeptide of an antibody.

In certain embodiments, each unique target polypeptide is a polypeptide of an antibody.

In certain embodiments, at least one unique target polypeptide is a polypeptide of a crossover dual-variable domain (CODV) Ig-like protein. In certain embodiments, at least one unique target polypeptide is a polypeptide of a crossover dual-variable domain (CODV) Ig-like protein triabody.

In certain embodiments, each unique target polypeptide is a polypeptide of a crossover dual-variable domain (CODV) Ig-like protein. In certain embodiments, each unique target polypeptide is a polypeptide of a crossover dual-variable domain (CODV) Ig-like protein triabody.

The recombinant mammalian host cell can be any mammalian cell or cell line capable of expressing the target polypeptide(s) in accordance with the methods and/or compositions disclosed herein. In certain embodiments, the recombinant mammalian host cell is selected from a CHO cell, a HEK-293 cell, BHK-21 cell, a HepG2 cell, a BAE-1 cell, an SH-SY5Y cell, a myeloma cell (e.g., Sp2/0-Ag14), a hybridoma cell, a HeLa cell, a Vero cell, an NIH3T3 cell, a WEHI231 cell, a YAC cell, a Jurkat cell, and derivatives thereof, for example a CHO-K1 cell and a CHO/DHFR⁻ cell. In certain embodiments, the recombinant mammalian host cell is a CHO cell.

### III. Compositions

A further aspect of the present disclosure is an engineered mammalian host cell comprising a plurality of recombinant polynucleotides, wherein each recombinant polynucleotide comprises a promoter, a nucleotide sequence encoding a unique cell surface marker polypeptide, and a nucleotide sequence encoding a unique target polypeptide, wherein both the nucleotide sequence encoding the unique cell surface marker polypeptide and the nucleotide sequence encoding the unique target polypeptide are transcribed on the same mRNA.

In certain embodiments, the plurality of unique target polypeptides comprises 2 to 8 unique target polypeptides.

In certain embodiments, the plurality of unique target polypeptides comprises 2 to 4 unique target polypeptides.

In certain embodiments, the plurality of unique target polypeptides comprises 2 unique target polypeptides.

In certain embodiments, the plurality of unique target polypeptides comprises 3 unique target polypeptides.

In certain embodiments, the plurality of unique target polypeptides comprises 4 unique target polypeptides.

In certain embodiments, each recombinant polynucleotide comprises, from 5′ to 3′, the promoter, the nucleotide sequence encoding the unique cell surface marker polypeptide, and the nucleotide sequence encoding the unique target polypeptide In certain embodiments, each recombinant polynucleotide further comprises an internal ribosome entry site (IRES) located 3′ to the nucleotide sequence encoding the unique cell surface marker polypeptide and 5′ to the nucleotide sequence encoding the unique target polypeptide.

In certain embodiments, each recombinant polynucleotide further comprises an alternate (non-ATG) start codon for translation initiation of the nucleotide sequence encoding the unique cell surface marker polypeptide and an ATG start codon for translation initiation of the nucleotide sequence encoding the unique target polypeptide.

In certain embodiments, each alternate (non-ATG) start codon is selected independently from the group consisting of CTG, GTG, TTG, ATT, ATA, and ACG.

In certain embodiments, each alternate (non-ATG) start codon is selected independently from the group consisting of GTG and TTG.

In certain embodiments, the nucleotide sequence encoding the unique cell surface marker polypeptide is devoid of any ATG triplets.

In certain embodiments, at least a first recombinant polynucleotide comprises, from 5′ to 3′, a first promoter, a nucleotide sequence encoding a first unique cell surface marker polypeptide, and a nucleotide sequence encoding a first unique target polypeptide; and at least a second recombinant polynucleotide comprises, from 5′ to 3′, a second promoter, a nucleotide sequence encoding a second unique target polypeptide, and a nucleotide sequence encoding a second unique cell surface marker polypeptide.

In certain embodiments, the first recombinant polynucleotide further comprises an internal ribosome entry site (IRES) located 3′ to the nucleotide sequence encoding the first unique cell surface marker polypeptide and 5′ to the nucleotide sequence encoding the first unique target polypeptide.

In certain embodiments, the first recombinant polynucleotide further comprises an alternate (non-ATG) start codon for translation initiation of the nucleotide sequence encoding the first unique cell surface marker polypeptide and an ATG start codon for translation initiation of the nucleotide sequence encoding the first unique target polypeptide.

In certain embodiments, the alternate (non-ATG) start codon is selected from the group consisting of CTG, GTG, TTG, ATT, ATA, and ACG.

In certain embodiments, the alternate (non-ATG) start codon is selected from the group consisting of GTG and TTG.

In certain embodiments, the nucleotide sequence encoding the first unique cell surface marker polypeptide is devoid of any ATG triplets.

In certain embodiments, the second recombinant polynucleotide further comprises an internal ribosome entry site (IRES) located 3′ to the nucleotide sequence encoding the second unique target polypeptide and 5′ to the nucleotide sequence encoding the second unique cell surface marker polypeptide.

In certain embodiments, each recombinant polynucleotide comprises, from 5′ to 3′, the promoter, the nucleotide sequence encoding the unique target polypeptide, and the nucleotide sequence encoding the unique cell surface marker polypeptide.

In certain embodiments, each recombinant polynucleotide further comprises an internal ribosome entry site (IRES) located 3′ to the nucleotide sequence encoding the unique target polypeptide and 5′ to the nucleotide sequence encoding the unique cell surface marker polypeptide.

In certain embodiments, at least one promoter is a β-actin promoter.

In certain embodiments, each promoter is a β-actin promoter.

In certain embodiments, at least one promoter is a hamster β-actin promoter.

In certain embodiments, each promoter is a hamster β-actin promoter.

In certain embodiments, at least a first unique cell surface marker polypeptide is selected from the group consisting of CD20, CD52, CD59, and variants thereof.

In certain embodiments, at least a second unique cell surface marker polypeptide is selected from the group consisting of CD20, CD52, CD59, and variants thereof.

In certain embodiments, each unique cell surface marker polypeptide is selected from the group consisting of CD20, CD52, CD59, and variants thereof.

In certain embodiments, at least a first unique cell surface marker polypeptide is selected from the group consisting of human CD52 and variants thereof.

In certain embodiments, at least a second unique cell surface marker polypeptide is selected from the group consisting of human CD52 and variants thereof.

In certain embodiments, each unique cell surface marker polypeptide is selected from the group consisting of human CD52 and variants thereof.

In certain embodiments, at least a first unique cell surface marker polypeptide is selected from the group consisting of CD20, CD52, and CD59.

In certain embodiments, at least a second unique cell surface marker polypeptide is selected from the group consisting of CD20, CD52, and CD59.

In certain embodiments, each unique cell surface marker polypeptide is selected from the group consisting of CD20, CD52, and CD59.

In certain embodiments, at least a first unique cell surface marker polypeptide is human CD52.

In certain embodiments, at least a second unique cell surface marker polypeptide is human CD52.

In certain embodiments, at least one unique cell surface marker polypeptide is a variant human CD52 comprising the amino acid sequence set forth in SEQ ID NO: 19 (GX₂NDTSQTSSPS) wherein X₂ is selected from the group consisting of A, G, I, L, and V. In certain embodiments, X₂ is A.

In certain embodiments, at least one unique cell surface marker polypeptide is a variant human CD52 comprising the amino acid sequence set forth in SEQ ID NO: 20 (GQNX₄TSQTSSPS) wherein X₄ is selected from the group consisting of A, G, I, L, and V. In certain embodiments, X₄ is A.

In certain embodiments, at least one unique cell surface marker polypeptide is a variant human CD52 comprising the amino acid sequence set forth in SEQ ID NO: 21 (GQNDTSX₇TSSPS) wherein X₇ is selected from the group consisting of A, G, I, L, and V. In certain embodiments, X₇ is A.

In certain embodiments, at least one unique cell surface marker polypeptide is a variant human CD52 comprising the amino acid sequence set forth in SEQ ID NO: 22 (GQNDTSQX₈SSPS) wherein X₈ is selected from the group consisting of A, G, I, L, and V. In certain embodiments, X₈ is A.

In certain embodiments, at least one unique cell surface marker polypeptide is a variant human CD52 comprising the amino acid sequence set forth in SEQ ID NO: 23 (GQNDTSQX₈X₉SPS), wherein each of X₈ and X₉ is independently selected from the group consisting of A, G, I, L, and V. In certain embodiments, each of X₈ and X₉ is A.

In certain embodiments, at least one unique cell surface marker polypeptide is a variant human CD52 comprising the amino acid sequence set forth in SEQ ID NO: 24 (GQNDTSQX₈SX₁₀PS), wherein each of X₈ and X₁₀ is independently selected from the group consisting of A, G, I, L, and V. In certain embodiments, each of X₈ and X₁₀ is A.

In certain embodiments, at least one unique cell surface marker polypeptide is a variant human CD52 comprising the amino acid sequence set forth in SEQ ID NO: 25 (GQNDTSQX₈X₉X₁₀PS), wherein each of X₈, X₉, and X₁₀ is independently selected from the group consisting of A, G, I, L, and V. In certain embodiments, each of X₈, X₉, and X₁₀ is A.

In certain embodiments, at least one unique cell surface marker polypeptide is a variant human CD52 comprising the amino acid sequence set forth in SEQ ID NO: 26 (GX₂NDTSQX₈X₉SPS), wherein each of X₂, X₈, and X₉ is independently selected from the group consisting of A, G, I, L, and V. In certain embodiments, each of X₂, X₈, and X₉ is A.

In certain embodiments, at least one unique cell surface marker polypeptide is a variant human CD52 comprising the amino acid sequence set forth in SEQ ID NO: 27 (GX₂NDTSQX₈SX₁₀PS), wherein each of X₂, X₈, and X₁₀ is independently selected from the group consisting of A, G, I, L, and V. In certain embodiments, each of X₂, X₈, and X₁₀ is A.

In certain embodiments, at least one unique cell surface marker polypeptide is a variant human CD52 comprising the amino acid sequence set forth in SEQ ID NO: 28 (GQNDTSX₇X₈X₉SPS), wherein each of X₇, X₈, and X₉ is independently selected from the group consisting of A, G, I, L, and V. In certain embodiments, each of X₇, X₈, and X₉ is A.

In certain embodiments, at least one unique cell surface marker polypeptide is a variant human CD52 comprising the amino acid sequence set forth in SEQ ID NO: 29 (GQNDTSX₇X₈SX₁₀PS), wherein each of X₇, X₈, and X₁₀ is independently selected from the group consisting of A, G, I, L, and V. In certain embodiments, each of X₇, X₈, and X₁₀ is A.

In certain embodiments, at least a first unique cell surface marker polypeptide comprises a variant human CD52 comprising the amino acid sequence set forth in SEQ ID NO: 19 (GX₂NDTSQTSSPS) wherein X₂ is selected from the group consisting of A, G, I, L, and V; and at least a second unique cell surface marker polypeptide comprises a variant human CD52 comprising an amino acid sequence selected from the group consisting of SEQ ID NO: 20 (GQNX₄TSQTSSPS) and SEQ ID NO: 21 (GQNDTSX₇TSSPS) wherein each of X₄ and X₇ is independently selected from the group consisting of A, G, I, L, and V.

In certain embodiments, at least a first unique cell surface marker polypeptide consists of a variant human CD52 consisting of the amino acid sequence set forth in SEQ ID NO: 19 (GX₂NDTSQTSSPS) wherein X₂ is selected from the group consisting of A, G, I, L, and V; and at least a second unique cell surface marker polypeptide consists of a variant human CD52 consisting of an amino acid sequence selected from the group consisting of SEQ ID NO: 20 (GQNX₄TSQTSSPS) and SEQ ID NO: 21 (GQNDTSX₇TSSPS) wherein each of X₄ and X₇ is independently selected from the group consisting of A, G, I, L, and V.

In certain embodiments, at least a first unique cell surface marker polypeptide comprises a variant human CD52 comprising the amino acid sequence set forth in SEQ ID NO: 2 (GANDTSQTSSPS); and at least a second unique cell surface marker polypeptide comprises a variant human CD52 comprising an amino acid sequence selected from the group consisting of SEQ ID NO: 4 (GQNATSQTSSPS) and SEQ ID NO: 7 (GQNDTSATSSPS).

In certain embodiments, at least a first unique cell surface marker polypeptide consists of a variant human CD52 consisting of the amino acid sequence set forth in SEQ ID NO: 2 (GANDTSQTSSPS); and at least a second unique cell surface marker polypeptide consists of a variant human CD52 consisting of an amino acid sequence selected from the group consisting of SEQ ID NO: 4 (GQNATSQTSSPS) and SEQ ID NO: 7 (GQNDTSATSSPS).

In certain embodiments, at least a first unique cell surface marker polypeptide comprises a variant human CD52 comprising the amino acid sequence set forth as SEQ ID NO: 2 (GANDTSQTSSPS); and at least a second unique cell surface marker polypeptide comprises a variant human CD52 comprising the amino acid sequence set forth as SEQ ID NO: 4 (GQNATSQTSSPS).

In certain embodiments, at least a first unique cell surface marker polypeptide consists of a variant human CD52 consisting of the amino acid sequence set forth as SEQ ID NO: 2 (GANDTSQTSSPS); and at least a second unique cell surface marker polypeptide consists of a variant human CD52 consisting of the amino acid sequence set forth as SEQ ID NO: 4 (GQNATSQTSSPS).

In certain embodiments, at least a first unique cell surface marker polypeptide comprises a variant human CD52 comprising the amino acid sequence set forth as SEQ ID NO: 2 (GANDTSQTSSPS); and at least a second unique cell surface marker polypeptide comprises a variant human CD52 comprising the amino acid sequence set forth as SEQ ID NO: 7 (GQNDTSATSSPS).

In certain embodiments, at least a first unique cell surface marker polypeptide consists of a variant human CD52 consisting of the amino acid sequence set forth as SEQ ID NO: 2 (GANDTSQTSSPS); and at least a second unique cell surface marker polypeptide consists of a variant human CD52 consisting of the amino acid sequence set forth as SEQ ID NO: 7 (GQNDTSATSSPS).

In certain embodiments, at least a first unique cell surface marker polypeptide comprises a variant human CD52 comprising the amino acid sequence set forth as SEQ ID NO: 4 (GQNATSQTSSPS); and at least a second unique cell surface marker polypeptide comprises a variant human CD52 comprising the amino acid sequence set forth as SEQ ID NO: 7 (GQNDTSATSSPS).

In certain embodiments, at least a first unique cell surface marker polypeptide comprises a variant human CD52 comprising the amino acid sequence set forth as SEQ ID NO: 20 (GQNX₄TSQTSSPS); at least a second unique cell surface marker polypeptide comprises a variant human CD52 comprising the amino acid sequence set forth as SEQ ID NO: 23 (GX₂NDTSQX₈X₉SPS); and at least a third unique cell surface marker polypeptide comprises a variant human CD52 comprising the amino acid sequence set forth as SEQ ID NO: 28 (GQNDTSX₇X₈X₉SPS), wherein each of X₄, X₇, X₈, and X₉ is independently selected from the group consisting of A, G, I, L, and V.

In certain embodiments, at least a first unique cell surface marker polypeptide comprises a variant human CD52 comprising the amino acid sequence set forth as SEQ ID NO: 20 (GQNX₄TSQTSSPS); at least a second unique cell surface marker polypeptide comprises a variant human CD52 comprising the amino acid sequence set forth as SEQ ID NO: 27 (GX₂NDTSQX₈SX₁₀PS); and at least a third unique cell surface marker polypeptide comprises a variant human CD52 comprising the amino acid sequence set forth as SEQ ID NO: 29 (GQNDTSX₇X₈SX₁₀PS) wherein each of X₄, X₇, X₈, and X₁₀ is independently selected from the group consisting of A, G, I, L, and V.

In certain embodiments, at least a first unique cell surface marker polypeptide comprises a variant human CD52 comprising the amino acid sequence set forth as SEQ ID NO: 4 (GQNATSQTSSPS); at least a second unique cell surface marker polypeptide comprises a variant human CD52 comprising the amino acid sequence set forth as SEQ ID NO: 15 (GANDTSQAASPS); and at least a third unique cell surface marker polypeptide comprises a variant human CD52 comprising the amino acid sequence set forth as SEQ ID NO: 17 (GQNDTSAAASPS).

In certain embodiments, at least a first unique cell surface marker polypeptide comprises a variant human CD52 comprising the amino acid sequence set forth as SEQ ID NO: 4 (GQNATSQTSSPS); at least a second unique cell surface marker polypeptide comprises a variant human CD52 comprising the amino acid sequence set forth as SEQ ID NO: 16 (GANDTSQASAPS); and at least a third unique cell surface marker polypeptide comprises a variant human CD52 comprising the amino acid sequence set forth as SEQ ID NO: 18 (GQNDTSAASAPS).

In certain embodiments, at least one unique target polypeptide comprises a therapeutic polypeptide or a therapeutic protein.

In certain embodiments, at least one unique target polypeptide is a polypeptide of a multi-chain protein.

In certain embodiments, each unique target polypeptide is a polypeptide of a multi-chain protein.

In certain embodiments, at least one unique target polypeptide is a polypeptide of an antibody.

In certain embodiments, each unique target polypeptide is a polypeptide of an antibody.

In certain embodiments, at least one unique target polypeptide is a polypeptide of a crossover dual-variable domain (CODV) Ig-like protein.

In certain embodiments, each unique target polypeptide is a polypeptide of a crossover dual-variable domain (CODV) Ig-like protein.

In certain embodiments, the recombinant mammalian host cell is a CHO cell.

A further aspect of the present disclosure is an isolated variant human CD52 comprising an amino acid sequence consisting of SEQ ID NO: 19 (GX₂NDTSQTSSPS) wherein X₂ is selected from the group consisting of A, G, I, L, and V. In certain embodiments, X₂ is A.

Yet a further aspect of the present disclosure is an isolated variant human CD52 comprising an amino acid sequence consisting of SEQ ID NO: 20 (GQNX₄TSQTSSPS) wherein X₄ is selected from the group consisting of A, G, I, L, and V. In certain embodiments, X₄ is A.

Yet a further aspect of the present disclosure is an isolated variant human CD52 comprising an amino acid sequence consisting of SEQ ID NO: 21 (GQNDTSQX₈SSPS) wherein X₈ is selected from the group consisting of A, G, I, L, and V. In certain embodiments, X₈ is A.

### EXAMPLES

### Example 1. Generation and Characterization of New Anti-hCD52 Monoclonal Antibodies

Mice were immunized with human CD52 (hCD52) and then 11 clones expressing murine monoclonal anti-hCD52 antibodies were prepared from binding antibodies using conventional methods. These 11 monoclonal antibodies were then converted into chimeric monoclonal antibodies having mouse variable regions and human constant regions, again using conventional methods. Affinities of the 11 clones on a hCD52 peptide mimetope was performed. The 11 clones are presented in Table 1.

TABLE 1
Anti-hCD52-expressing clones

| | original | converted | |
|---|---|---|---|
| | (mouse) | (human) | |
| mAb | isotype | isotype | Kd (nM)* |
| 7F11 | mIgG₁ | hIgG₁ | ND |
| 4B10 | mIgG_{2A} | hIgG₁ | ND |
| 2C3 | mIgG₃ | hIgG₁ | 4.0 |
| 12G6 | mIgG₃ | hIgG₁ | 4.2 |
| 23E6 | mIgG₃ | hIgG₁ | 6.1 |
| 9D9 | mIgG₃ | hIgG₁ | 43 |
| 11C11 | mIgG₃ | hIgG₁ | 70 |
| 3G7 | mIgG_{2B} | hIgG₁ | 79 |
| 4G7 | mIgG₃ | hIgG₁ | 101 |
| 8G3 | mIgG₃ | hIgG₁ | 127 |
| 5F7 | mIgG₃ | hIgG₁ | 260 |

ND, not done
*Campath-1H has a measured Kd of 68 nM.

Next, the clones were characterized in terms of ELISA-based binding profile on truncated and alanine scanning series of hCD52 peptide mimetopes. Comparison was made to Campath-1H (C-1H). Results are shown in Tables 2 and 3. Table 2 shows that C-1H binds predominantly to the C-terminal region of hCD52, whereas 9D9, 11C11, 3G7, and CF1D12 bind predominantly to the N-terminal region of hCD52. These results are largely consistent with the data shown in Table 3, i.e., alanine scanning in the C-terminal region of hCD52 abrogated binding by C-1H, whereas alanine scanning in N-terminal region of hCD52 abrogated binding by 9D9, 11C11, and 3G7.

Certain of these clones (antibodies) were also characterized in terms of their FACS-based binding profile. Briefly, hCD52 and individual alanine scanning variants of hCD52 corresponding to the peptide mimetopes in Table 3 were separately expressed by CHO cells, which were then analyzed by FACS using the selected antibodies described in this example (4B10, 7F11, CF1D12, 5F7, 3G7, 4G7, 9D9, 11C11, Campath-1H, 2C3, 12G8, and 23E6). The amino acid sequences of the test antigens were as follows:

| | | |
|---|---|---|
| Mut1 | AQNDTSQTSSPS | SEQ ID NO: 1 |
| Mut2 | GANDTSQTSSPS | SEQ ID NO: 2 |
| Mut3 | GQADTSQTSSPS | SEQ ID NO: 3 |
| Mut4 | GQNATSQTSSPS | SEQ ID NO: 4 |
| Mut5 | GQNDASQTSSPS | SEQ ID NO: 5 |
| Mut6 | GONDTAQTSSPS | SEQ ID NO: 6 |
| Mut7 | GONDTSATSSPS | SEQ ID NO: 7 |
| Mut8 | GQNDTSQASSPS | SEQ ID NO: 8 |
| Mut9 | GQNDTSQTASPS | SEQ ID NO: 9 |
| Mut10 | GQNDTSQTSAPS | SEQ ID NO: 10 |
| WT | GQNDTSQTSSPS | SEQ ID NO: 11 |

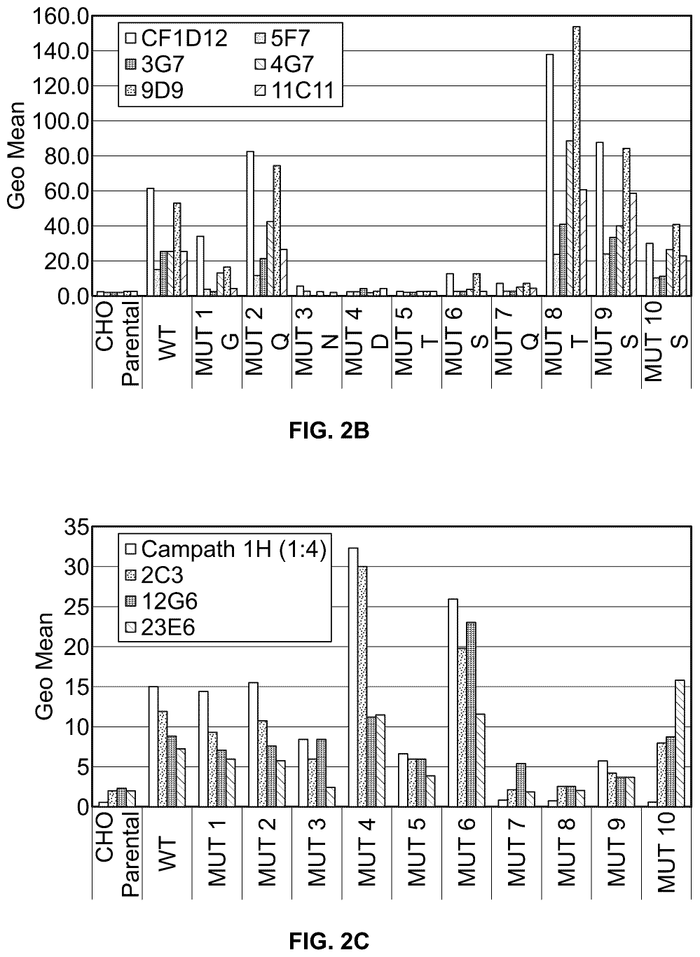

Results are shown in FIGS. 2A-2C, which show that Campath-1H and clone 9D9 clearly discriminated between cell surface-presented hCD52 alanine mutants 4 (GQNATSQTSSPS; SEQ ID NO: 4) and 8 (GQNDTSQASSPS; SEQ ID NO: 8).

The results of these studies provided proof of principal for the potential to develop a system for monitoring, in individual cells, co-expression of a plurality of different target polypeptides by assessing expression of corresponding unique cell surface marker polypeptides.

TABLE 2
ELISA-based binding profile on truncated series of hCD52 peptide mimetopes

| | SEQ | | | | | | | | | | | | | |
|---|---|---|---|---|---|---|---|---|---|---|---|---|---|---|
| | ID | | | | | | | | | | | | | |
| Peptide | NO: | 3G7 | 4G7 | 9D9 | 11C11 | 8G3 | 7F11 | 4B10 | 12G6 | 2C3 | 23E6 | 5F7 | C-1H | CF1D12 |
| GQNDTSQTSSPSAD | 39 | + | + | + | + | + | − | − | + | + | + | + | + | + |
| QNDTSQTSSPSAD | 40 | − | + | − | − | − | − | − | + | + | + | − | + | − |
| NDTSQTSSPSAD | 41 | − | + | − | − | − | − | − | + | + | + | − | + | − |
| DTSQTSSPSAD | 42 | − | + | − | − | − | − | − | + | + | + | − | + | − |
| TSQTSSPSAD | 43 | − | + | − | − | − | − | − | + | + | + | − | + | − |
| SQTSSPSAD | 44 | − | + | − | − | − | − | − | + | + | + | − | + | − |
| QTSSPSAD | 45 | − | + | − | − | − | − | − | + | + | + | − | + | − |
| TSSPSAD | 46 | − | + | − | − | − | − | − | − | − | − | − | − | + |
| GQNDTSQTSSPAD | 47 | + | + | + | + | + | − | − | + | + | + | + | − | + |
| GQNDTSQTSSAD | 48 | + | + | + | + | − | − | − | − | − | − | − | − | + |
| GQNDTSQTSAD | 49 | + | + | + | + | − | − | − | − | − | − | − | − | + |
| GQNDTSQTAD | 50 | + | − | + | + | − | − | − | − | − | − | − | − | + |
| GQNDTSQAD | 51 | + | + | + | + | − | − | − | − | − | − | + | − | + |
| GQNDTSAD | 52 | − | + | + | + | − | − | − | − | − | − | − | − | + |
| GQNDTAD | 53 | − | − | − | − | − | − | − | − | − | − | − | − | + |

TABLE 3
ELISA-based binding profile on alanine scanning series of hCD52 peptide mimetopes

| | SEQ | | | | | | | | | | | | | |
|---|---|---|---|---|---|---|---|---|---|---|---|---|---|---|
| | ID | | | | | | | | | | | | | |
| Peptide | NO: | 3G7 | 4G7 | 9D9 | 11C11 | 8G3 | 7F11 | 4B10 | 12G6 | 2C3 | 23E6 | 5F7 | C-1H | CF1D12 |
| GQNDTSQTSSPSAD | 39 | + | + | + | + | + | − | − | + | + | + | + | + | + |
| AQNDTSQTSSPSAD | 54 | − | + | − | − | − | − | − | + | + | + | − | + | + |
| GANDTSQTSSPSAD | 55 | + | + | + | + | + | − | − | + | + | + | + | + | + |
| GQADTSQTSSPSAD | 56 | − | + | − | − | − | − | − | + | + | + | − | + | + |
| GQNATSQTSSPSAD | 57 | − | − | − | − | − | − | − | + | + | + | − | + | − |
| GQNDASQTSSPSAD | 58 | − | − | − | − | − | − | − | + | + | + | − | + | + |
| GQNDTAQTSSPSAD | 59 | − | − | − | − | − | − | − | + | + | + | − | + | + |
| GQNDTSATSSPSAD | 60 | − | + | − | + | − | − | − | − | − | − | − | + | + |
| GQNDTSQASSPSAD | 61 | + | + | + | + | + | − | − | + | + | + | − | + | + |
| GQNDTSQTASPSAD | 62 | + | + | + | + | + | − | − | + | + | + | + | − | + |
| GQNDTSQTSAPSAD | 63 | + | + | + | + | + | − | − | + | + | + | + | − | + |
| GQNDTSQTSSASAD | 64 | + | + | + | + | − | − | − | + | + | + | − | − | + |
| GQNDTSQTSSPAAD | 65 | + | + | + | + | + | − | − | + | + | + | + | − | + |

### Example 2. Polynucleotide Constructs Encoding Exemplary hCD52 Mutants Useful as Unique Cell Surface Marker Polypeptides

Polynucleotides encoding various mutant versions of hCD52 including a non-AUG (alternate) start codon with optimal Kozak context, mutation of all internal ATG codons, eliminated splice sites, and a single point mutation substituting alanine for selected amino acids of hCD52 were prepared.

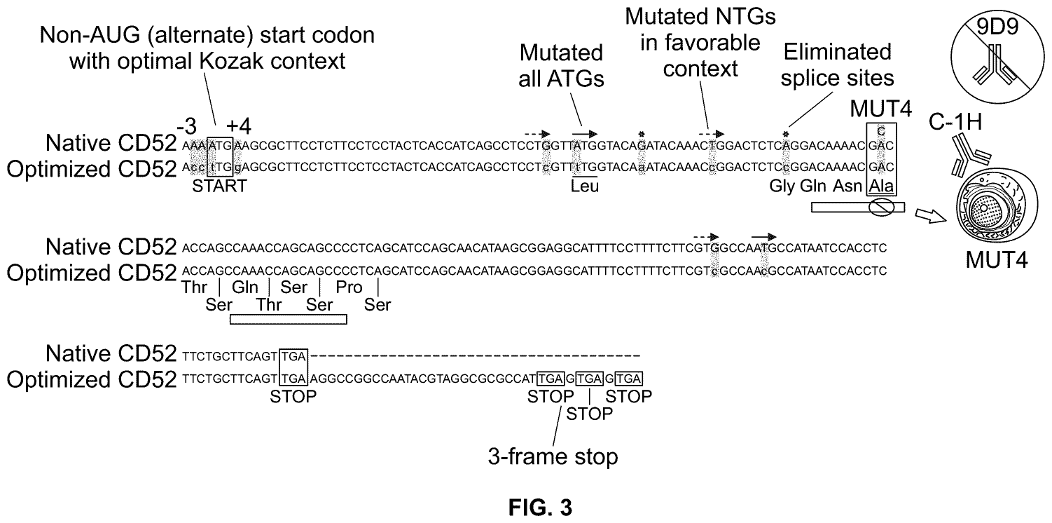

FIG. 3 depicts a sequence alignment of native (wild-type) hCD52 (SEQ ID NO: 30) and engineered mutant hCD52 (Mut4; SEQ ID NO: 31) including a non-AUG (alternate) start codon with optimal Kozak context, mutation of all internal ATG codons, eliminated splice sites, and a single point mutation substituting alanine for aspartic acid at amino acid 4 of hCD52. Mut4 is bound by Campath-1H (C-1H) but not by mAb 9D9.

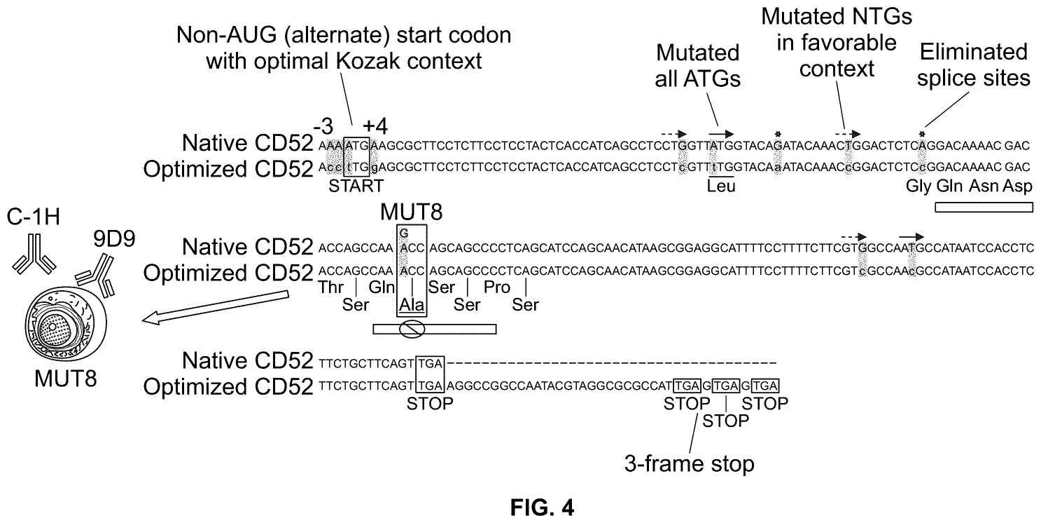

FIG. 4 depicts a sequence alignment of native (wild-type) hCD52 (SEQ ID NO: 30) and engineered mutant hCD52 (Mut8; SEQ ID NO: 32) including a non-AUG (alternate) start codon with optimal Kozak context, mutation of all internal ATG codons, eliminated splice sites, and a single point mutation substituting alanine for threonine at amino acid 8 of hCD52. Mut8 is bound by mAb 9D9 but not by Campath-TH (C-1H).

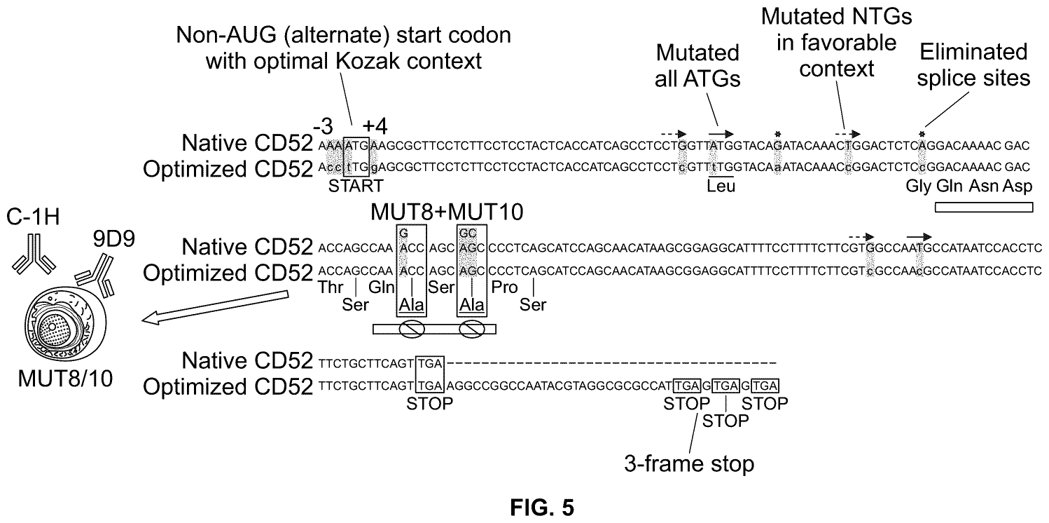

FIG. 5 depicts a sequence alignment of native (wild-type) hCD52 (SEQ ID NO: 30) and engineered double mutant hCD52 (Mut8/10; SEQ ID NO: 33) including a non-AUG (alternate) start codon with optimal Kozak context, mutation of all internal ATG codons, eliminated splice sites, and two point mutations substituting alanine for threonine at amino acid 8 of hCD52 and alanine for serine at amino acid 10 of hCD52. Similar to Mut8, Mut8/10 is bound by mAb 9D9 but not by Campath-1H (C-1H).

### Example 3. Additional Polynucleotide Constructs Encoding Exemplary hCD52 Mutants Useful as Unique Cell Surface Marker Polypeptides

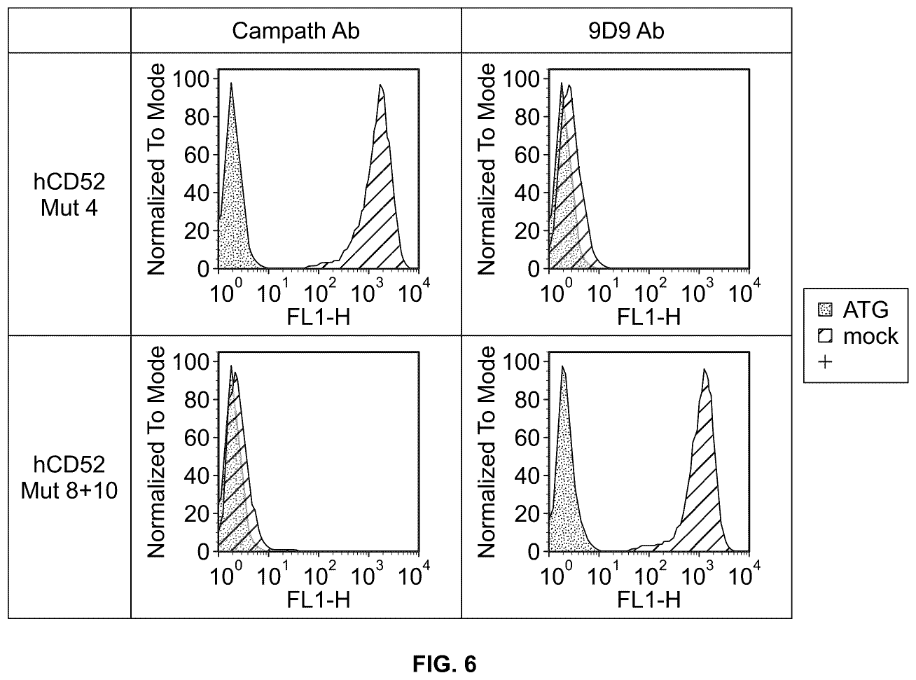

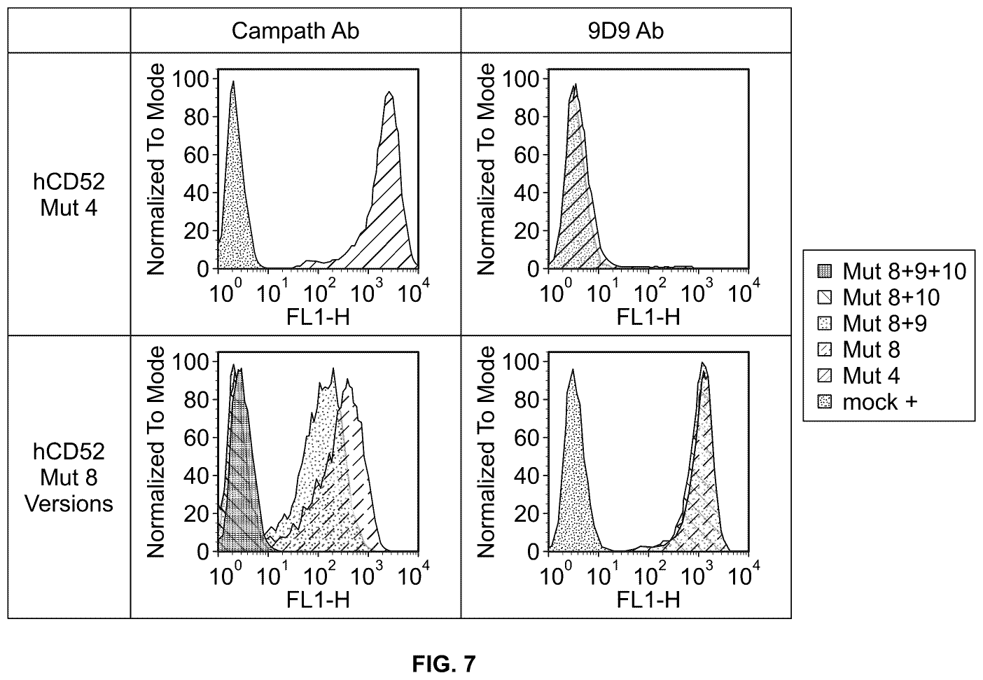

CHO cells were transfected with polynucleotides encoding the following mutants of hCD52: Mut4, Mut8, Mut8/9, Mut8/10, and Mut8/9/10. All mutants had ATG start codons. Cells were assayed by FACS using fluorophore-labeled Campath-1H and 9D9 antibodies. Results are shown in FIG. 6 and FIG. 7.

FIG. 6 is four flow cytometry histograms depicting binding by Campath-1H or 9D9 to cells expressing Mut4 (SEQ ID NO: 4) or Mut8/10 (SEQ ID NO: 13) of hCD52, where the polynucleotide encoding each mutant of hCD52 has an ATG start codon. The figure indicates that Mut4 mutants and Mut8/10 mutants were readily distinguishable using Campath-1H and 9D9.

FIG. 7 is four flow cytometry histograms depicting binding by Campath-1H or 9D9 to cells expressing Mut4 (SEQ ID NO: 4), Mut8 (SEQ ID NO: 8), Mut8/9 (SEQ ID NO: 12), Mut8/10 (SEQ ID NO: 13), or Mut8/9/10 (SEQ ID NO: 14) of hCD52, where the polynucleotide encoding each mutant of hCD52 has an ATG start codon. The figure indicates that Mut4 mutants and all four Mut8-containing mutants were readily distinguishable using Campath-1H and 9D9.

### Example 4. Effect of Non-ATG Start Codon Usage

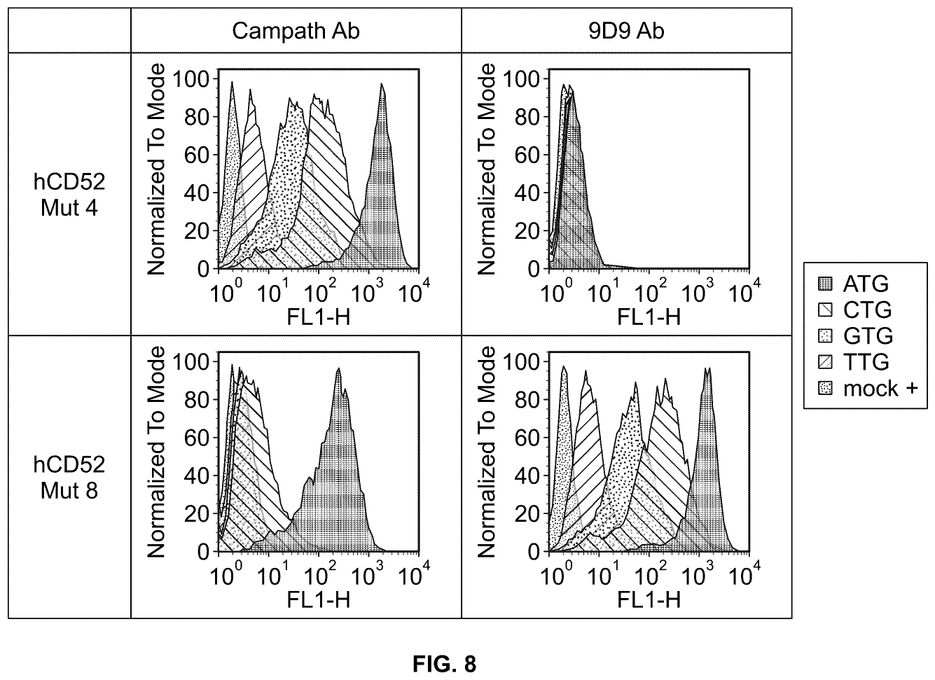

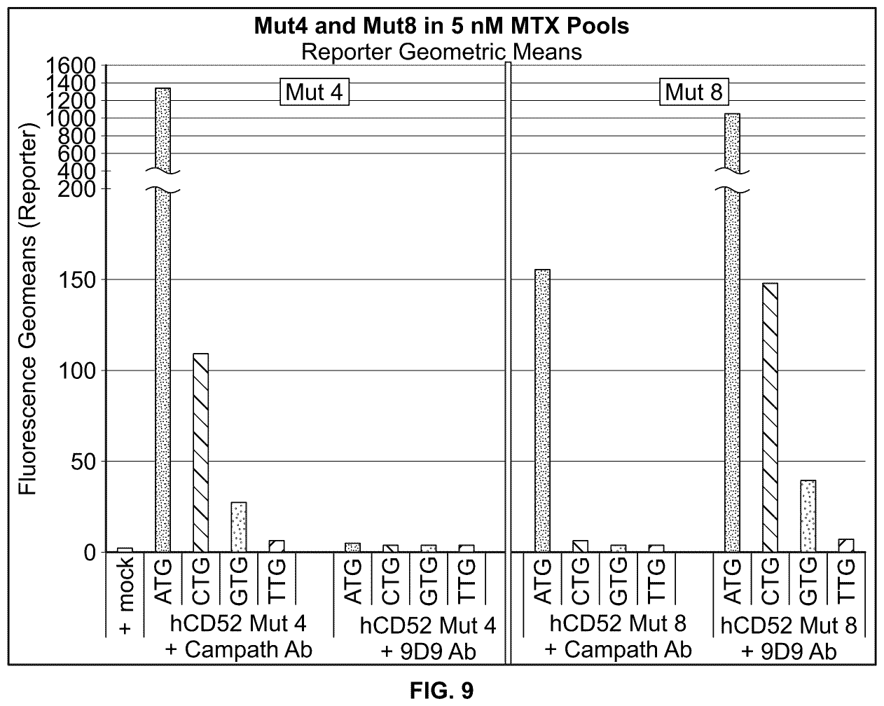

CHO cells were transfected with polynucleotides encoding hCD52 mutants Mut4 and Mut8, each with ATG, CTG, GTG, or TTG start codons. Cells were assayed by FACS using fluorophore-labeled Campath-1H and 9D9 antibodies. Results are shown in FIG. 8 and FIG. 9.

FIG. 8 is four flow cytometry histograms depicting binding by Campath-1H or 9D9 to cells expressing Mut4 (SEQ ID NO: 4) or Mut8 (SEQ ID NO: 8), individually according to start codon. The figure shows that expression for each of Mut4 and Mut8 followed the pattern ATG>CTG>GTG>TTG>mock.

FIG. 9 is a bar graph representation of data from FIG. 8, showing that Mut4 and Mut8 mutants, especially with ATG, CTG, or GTG start codons, were readily distinguishable using Campath-1H and 9D9.

### Example 5. Effect of Cell Surface Marker Start Codon on Expression of Target Polypeptide

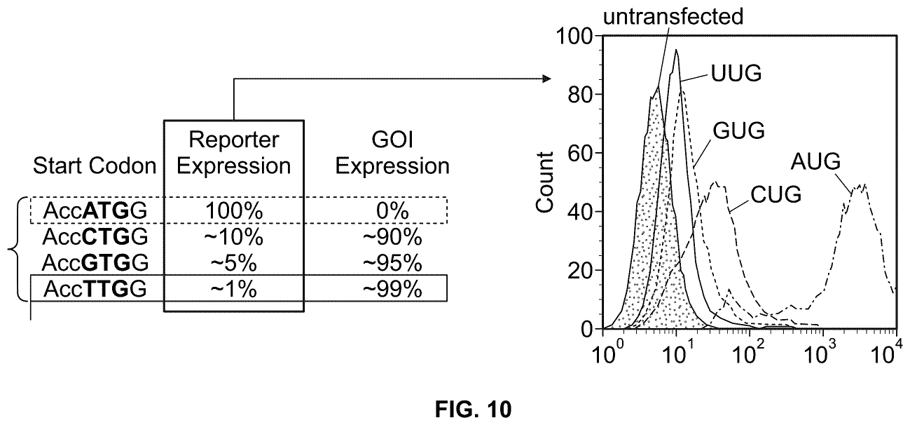

Stably transfected CHO pools co-expressing an alternate start reporter and a soluble receptor-Fc fusion protein were analyzed using flow cytometric methods. Each reporter was expressed using UUG, GUG, CUG, or AUG as the initiation codon. Histogram overlays show the level of cell surface expression of CD52 or CD59 in which all AUGs 3′ of the initiation codon were mutated. Cairns et al. (2011) Biotechnol. Bioeng. 108: 2611-22. Representative results are shown in FIG. 10. The figure shows that there was an inverse relationship between reporter (cell surface marker) expression and gene of interest (GOI; target polypeptide) expression, which varied according to start codon in the polynucleotide encoding the reporter. Results showed that TTG demonstrated the lowest detectable level of CD52 expression which would allow a greater read-through of ribosomes to the GOI. Since ultimately it is expression of target protein, rather than reporter, that is important, the figure shows that selection of a sub-optimal non-ATG start codon such as TTG is sufficient and to be preferred.

### Example 6. hCD52 Alanine Mutant Combinations that can Enable Three Reporters

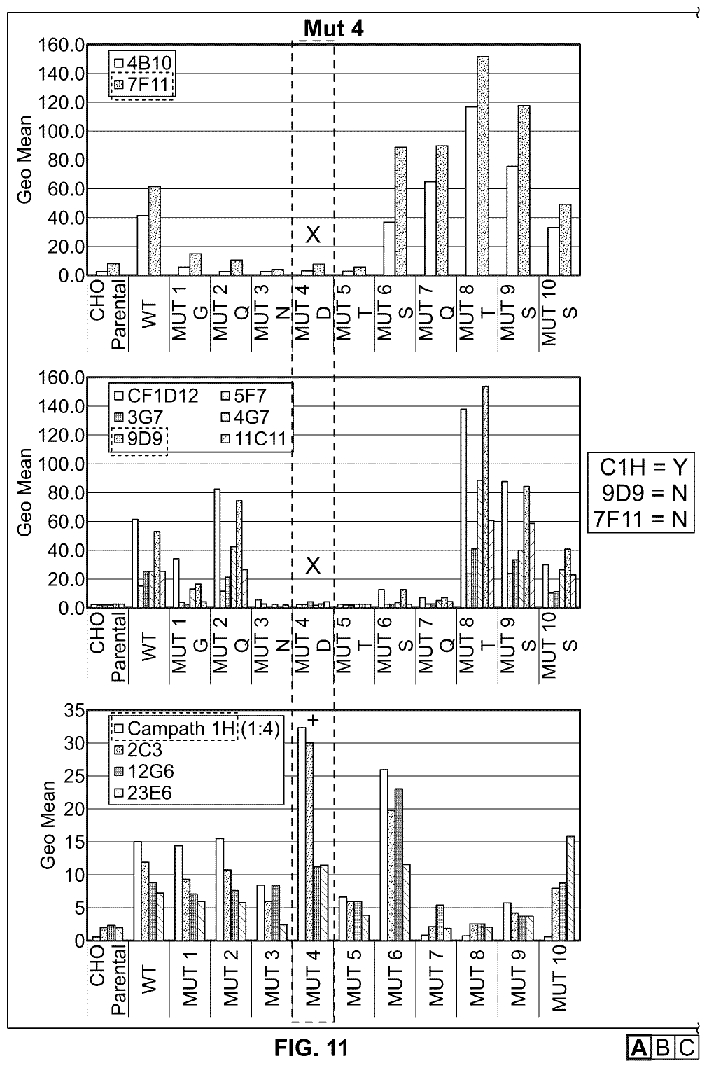

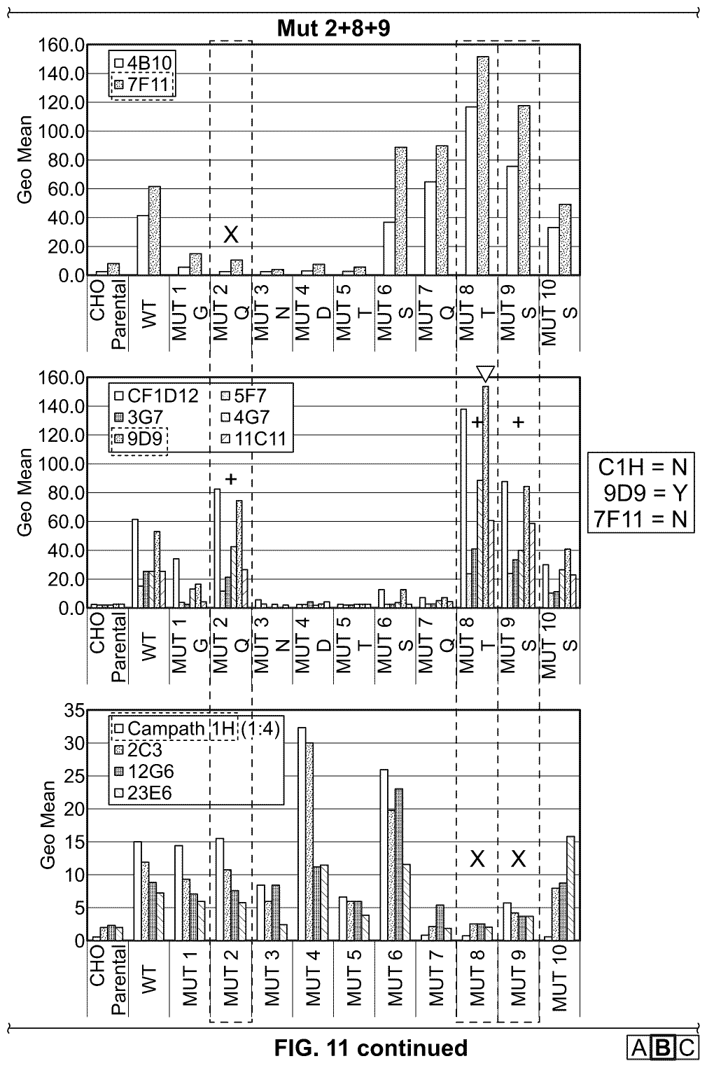

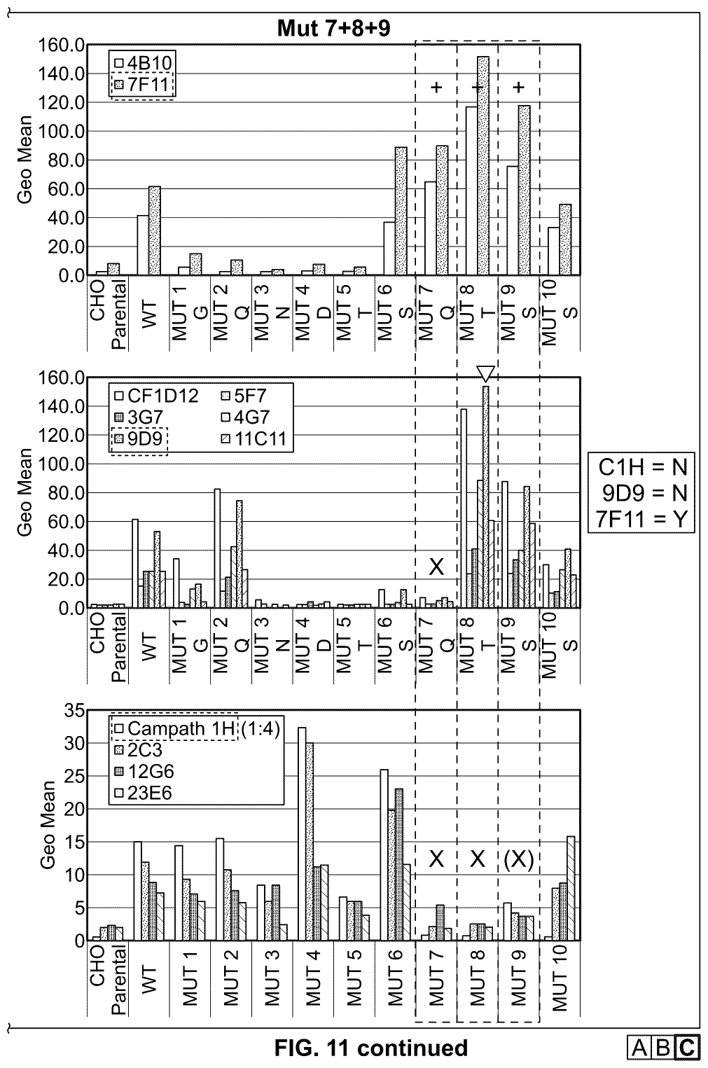

As further proof of concept, FIG. 11 illustrates how particular combinations of certain hCD52 alanine mutants can enable three unique reporters. As shown in FIG. 11, hCD52 mutant 4 (GQNATSQTSSPS; SEQ ID NO: 4) was identified with Campath-1H (C-1H) but not with 9D9 or 7F11. hCD52 mutant Mut2/8/9 (GANDTSQAASPS; SEQ ID NO: 15) was identified by binding to 9D9 but not to C1H or 7F111. hCD52 mutant Mut7/8/9 (GQNDTSAAASPS; SEQ ID NO: 17) was identified by binding to 7F111 but not to C1H or 9D9.

### Example 7. Additional hCD52 Alanine Mutant Combinations that can Enable Three Reporters

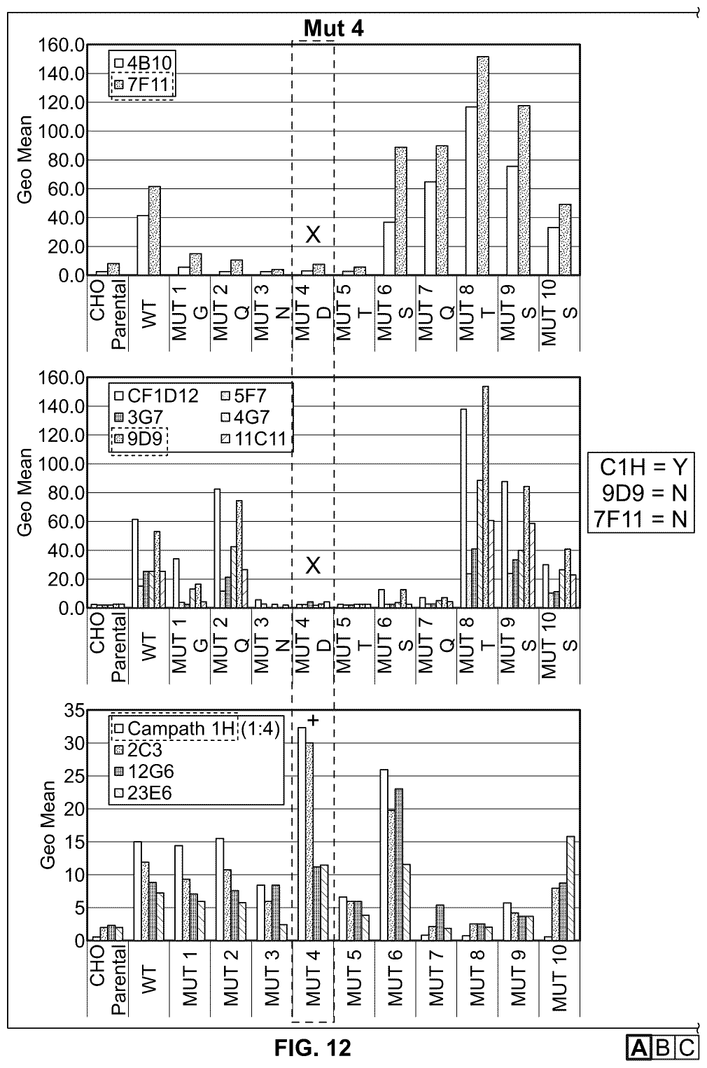

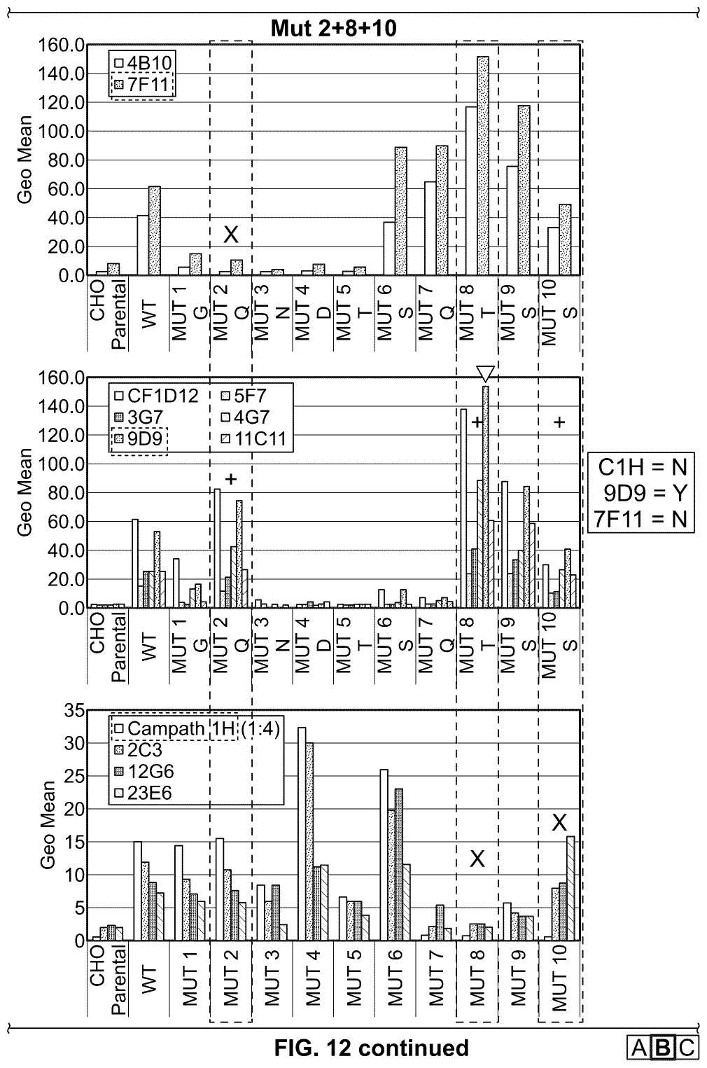

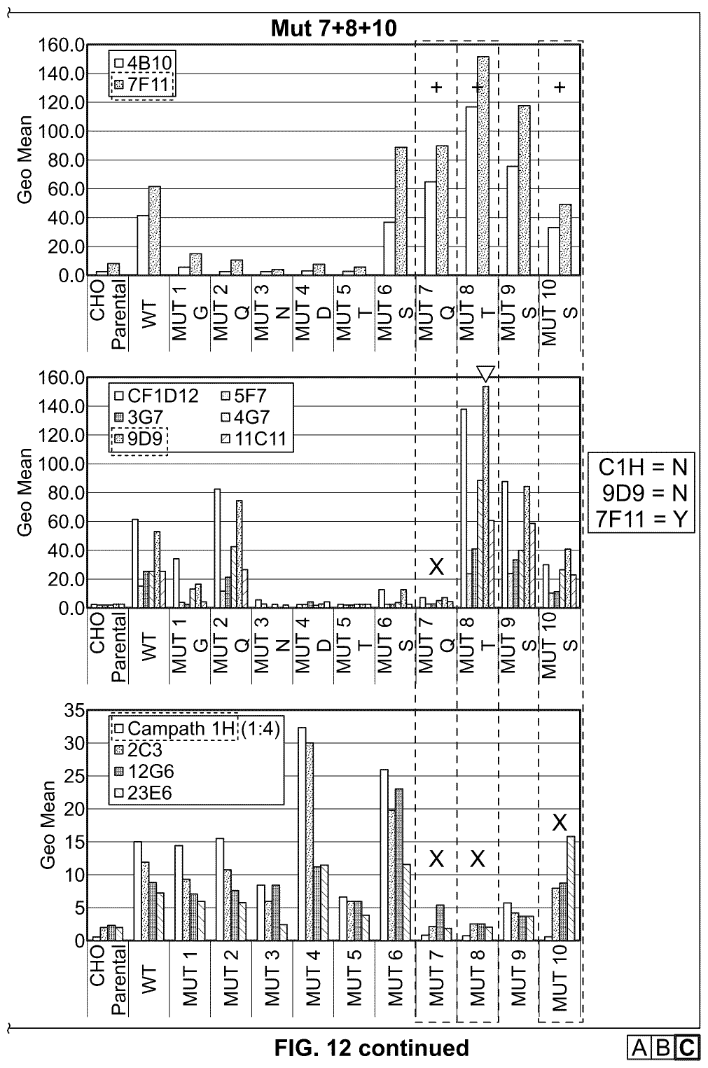

As yet further proof of concept, FIG. 12 illustrates how particular combinations of certain hCD52 alanine mutants can enable three unique reporters. As shown in FIG. 12, hCD52 mutant 4 (GQNATSQTSSPS; SEQ ID NO: 4) was identified with Campath-1H (C-1H) but not with 9D9 or 7F111. hCD52 mutant Mut2/8/10 (GANDTSQASAPS; SEQ ID NO: 16) was identified by binding to 9D9 but not to C1H or 7F11. hCD52 mutant Mut7/8/9 (GQNDTSAASAPS; SEQ ID NO: 18) was identified by binding to 7F11 but not to C1H or 9D9.

### Example 8. Dual Reporter Systems for Use in CODV Triabody Expression

While CODV triabodies have been described, their production is extremely challenging. These constructs have four different polypeptide chains: two different heavy chains (CODV heavy chain and Fab heavy chain, with reciprocal knobs and holes) and two different light chains (CODV light chain and Fab light chain). Balancing expression of these four individual polypeptide chains is difficult, and excess free heavy chains can be toxic to cells expressing them.

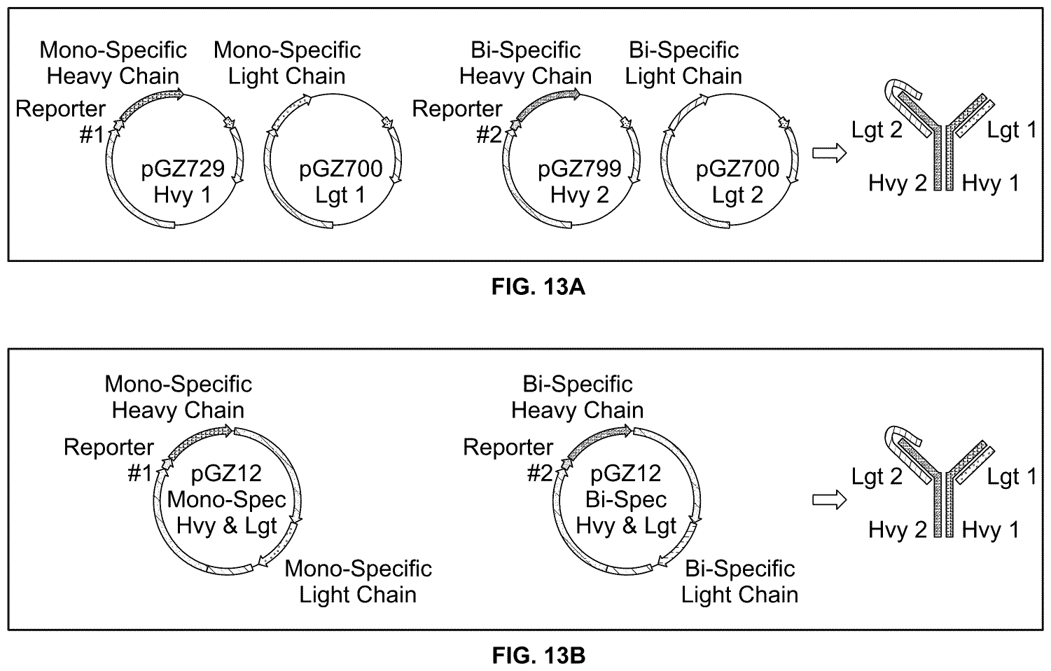

In one embodiment, each heavy chain is expressed from its own separate expression vector together with a unique cell surface marker polypeptide encoded on a single mRNA, and each light chain is expressed without any unique cell surface marker polypeptide from its own separate expression vector. The corresponding heavy and light chains (CODV heavy and light chains, and Fab heavy and light chains) self-associate to form the desired CODV triabody. See FIG. 13A.

Notably, this method provides the ability to isolate and screen clones for each arm independently, and it may provide a desirable screen for product quality (i.e., homogeneous tri-specific expression.

In another embodiment, a similar approach can be taken with the exception that corresponding heavy and light chains are encoded on a single bi-cistronic vector. See FIG. 13B.

1. A method for expressing a plurality of target polypeptides at a high level, the method comprising:
(a) culturing a plurality of mammalian host cells, each comprising a plurality of recombinant polynucleotides, wherein
each recombinant polynucleotide comprises a promoter, a nucleotide sequence encoding a unique cell surface marker polypeptide, and a nucleotide sequence encoding a unique target polypeptide, wherein both the nucleotide sequence encoding the unique cell surface marker polypeptide and the nucleotide sequence encoding the unique target polypeptide are transcribed on the same mRNA,
wherein the culturing is under conditions that permit expression of each unique cell surface marker polypeptide on the surface of the mammalian host cells and expression of each unique target polypeptide;
(b) contacting the cultured mammalian host cells of step (a) with a plurality of detectable agents, each uniquely capable of binding one or more of the unique cell surface marker polypeptides expressed on the surface of the mammalian host cells;
(c) performing at least one round of fluorescence-activated cell sorting on the contacted cells from step (b), thereby selecting one or more mammalian host cells that are uniquely bound by at least one of the plurality of detectable agents;
(d) preparing one or more clonal populations of the mammalian host cells selected in step (c);
(e) analyzing one or more clonal populations from step (d) by detecting level of expression of at least two unique cell surface marker polypeptides on each of said clonal populations;
(f) selecting one or more clonal populations having high expression level of the at least two unique cell surface marker polypeptides; and
(g) culturing one or more clonal populations selected in step (f) under conditions that permit expression of the plurality of target polypeptides at a high level.

2. The method of claim 1, wherein step (c) comprises performing a single round of fluorescence-activated cell sorting on the contacted cells from step (b) with at least a first detectable agent and a second detectable agent, thereby selecting one or more mammalian host cells that are uniquely bound by at least both the first detectable agent and the second detectable agent; or
wherein step (c) comprises
(c1) performing a first round of fluorescence-activated cell sorting on at least a first cell surface marker polypeptide, thereby selecting one or more mammalian host cells that are bound by at least a first detectable agent; and
(c2) performing a second round of fluorescence-activated cell sorting, on cells selected in step (c1), on at least a second cell surface marker polypeptide, thereby selecting one or more mammalian host cells that are bound by at least both the first detectable agent and the second detectable agent.

3. (canceled)

4. The method of claim 1, wherein:
(a) the step (e) is performed 7-28 days after step (d),
(b) the analyzing in step (e) comprises flow cytometry;
(c) the expression level of at least one of the plurality of unique cell surface marker polypeptides in step (f) is higher than a corresponding expression level of at least 70% of the clonal populations analyzed in step (e);
(d) the expression level of each of the plurality of unique cell surface marker polypeptides in step (f) is higher than a corresponding expression level of at least 70% of the clonal populations analyzed in step (e);
(e) the expression level of at least a first of the plurality of unique cell surface polypeptides in step (f) is higher than the expression level of at least a second of the plurality of unique cell surface polypeptides in step (f); and/or
(f) wherein the expression level of a first of the plurality of unique cell surface polypeptides in step (f) is higher than the expression level of a second of the plurality of unique cell surface polypeptides in step (f).

5-9. (canceled)

10. The method of claim 1, further comprising:
(h) isolating at least a first unique target polypeptide and a second unique target polypeptide expressed in step (g) from the one or more selected clonal populations or from cell culture medium in which the one or more selected clonal populations are cultured.

11. The method of claim 1, wherein the plurality of unique target polypeptides comprises 2 to 8 unique target polypeptides, optionally wherein the plurality of unique target polypeptides comprises 2 to 4 unique target polypeptides, the plurality of unique target polypeptides comprises 2 unique target polypeptides, the plurality of unique target polypeptides comprises 3 unique target polypeptides, or the plurality of unique target polypeptides comprises 4 unique target polypeptides.

12-15. (canceled)

16. The method of claim 1, wherein
each recombinant polynucleotide comprises, from 5′ to 3′, the promoter, the nucleotide sequence encoding the unique cell surface marker polypeptide, and the nucleotide sequence encoding the unique target polypeptide;
each recombinant polynucleotide comprises 1 to 4 promoters, 1 to 4 nucleotide sequences each encoding a unique cell surface marker polypeptide, and 1 to 4 nucleotide sequences each encoding a unique target polypeptide, wherein one of the nucleotide sequences encoding a unique cell surface marker polypeptide is transcribed on the same mRNA as one of the nucleotide sequences encoding a unique target polypeptide;
each recombinant polynucleotide is a bicistronic polynucleotide comprising two promoters and two nucleotide sequences each encoding a unique target polypeptide, and further comprising one or two nucleotide sequences each encoding a unique cell surface marker polypeptide;
each recombinant polynucleotide is a bicistronic polynucleotide comprising two promoters and two nucleotide sequences each encoding a unique target polypeptide, and further comprising one or two nucleotide sequences each encoding a unique cell surface marker polypeptide;
each bicistronic polynucleotide comprises a single nucleotide sequence encoding a unique cell surface marker polypeptide;
each bicistronic polynucleotide comprises two nucleotide sequences each encoding a unique cell surface marker polypeptide, and wherein each one of the nucleotide sequences encoding a unique cell surface marker polypeptide is transcribed on the same mRNA as one of the nucleotide sequences encoding a unique target polypeptide;
each recombinant polynucleotide comprises, from 5′ to 3′, one of the from 1-4 promoters, one of the from 1-4 nucleotide sequences encoding a unique cell surface marker polypeptide, and one of the from 1-4 nucleotide sequences encoding a unique target polypeptide;
each recombinant polynucleotide comprises one promoter, one nucleotide sequence encoding a unique cell surface marker polypeptide, and one nucleotide sequence encoding a unique target polypeptide, wherein the nucleotide sequence encoding a unique cell surface marker polypeptide is transcribed on the same mRNA as the nucleotide sequence encoding a unique target polypeptide;
each recombinant polynucleotide further comprises an internal ribosome entry site (IRES) located 3′ to the nucleotide sequence encoding the unique cell surface marker polypeptide and 5′ to the nucleotide sequence encoding the unique target polypeptide; and/or
each recombinant polynucleotide further comprises an alternate (non-ATG) start codon for translation initiation of the nucleotide sequence encoding the unique cell surface marker polypeptide and an ATG start codon for translation initiation of the nucleotide sequence encoding the unique target polypeptide, optionally wherein each alternate (non-ATG) start codon is selected independently from the group consisting of CTG, GTG, TTG, ATT, ATA, and ACG, each alternate (non-ATG) start codon is selected independently from the group consisting of GTG and TTG, or wherein the nucleotide sequence encoding the unique cell surface marker polypeptide is devoid of any ATG triplets.

17-27. (canceled)

28. The method of claim 1, wherein
at least a first recombinant polynucleotide comprises, from 5′ to 3′, a first promoter, a nucleotide sequence encoding a first unique cell surface marker polypeptide, and a nucleotide sequence encoding a first unique target polypeptide optionally wherein
the first recombinant polynucleotide further comprises an internal ribosome entry site (IRES) located 3′ to the nucleotide sequence encoding the first unique cell surface marker polypeptide and 5′ to the nucleotide sequence encoding the first unique target polypeptide, or
the first recombinant polynucleotide further comprises an alternate (non-ATG) start codon for translation initiation of the nucleotide sequence encoding the first unique cell surface marker polypeptide and an ATG start codon for translation initiation of the nucleotide sequence encoding the first unique target polypeptide; and
at least a second recombinant polynucleotide comprises, from 5′ to 3′, a second promoter, a nucleotide sequence encoding a second unique target polypeptide, and a nucleotide sequence encoding a second unique cell surface marker polypeptide, optionally wherein
the second recombinant polynucleotide further comprises an internal ribosome entry site (IRES) located 3′ to the nucleotide sequence encoding the second unique target polypeptide and 5′ to the nucleotide sequence encoding the second unique cell surface marker polypeptide; and/or
wherein each recombinant polynucleotide comprises, from 5′ to 3′, the promoter, the nucleotide sequence encoding the unique target polypeptide, and the nucleotide sequence encoding the unique cell surface marker polypeptide, optionally wherein each recombinant polynucleotide further comprises an internal ribosome entry site (IRES) located 3′ to the nucleotide sequence encoding the unique target polypeptide and 5′ to the nucleotide sequence encoding the unique cell surface marker polypeptide.

29-36. (canceled)

37. The method of claim 1, wherein at least one promoter is a β-actin promoter, each promoter is a β-actin promoter, at least one promoter is a hamster β-actin promoter, or each promoter is a hamster β-actin promoter.

38-40. (canceled)

41. The method of claim 1, wherein
at least a first unique cell surface marker polypeptide is selected from the group consisting of CD20, CD52, CD59, and variants thereof;
at least a second unique cell surface marker polypeptide is selected from the group consisting of CD20, CD52, CD59, and variants thereof;
each unique cell surface marker polypeptide is selected from the group consisting of CD20, CD52, CD59, and variants thereof;
at least a first unique cell surface marker polypeptide is selected from the group consisting of human CD52 and variants thereof;
at least a second unique cell surface marker polypeptide is selected from the group consisting of human CD52 and variants thereof;
each unique cell surface marker polypeptide is selected from the group consisting of human CD52 and variants thereof;
at least a first unique cell surface marker polypeptide is human CD52; and/or
at least a second unique cell surface marker polypeptide is human CD52.

42-51. (canceled)

52. The method of claim 1, wherein at least one unique cell surface marker polypeptide is a variant human CD52 comprising:
(a) the amino acid sequence set forth in SEQ ID NO: 19 (GX₂NDTSQTSSPS) wherein X₂ is selected from the group consisting of A, G, I, L, and V, optionally wherein X₂ is A;
(b) the amino acid sequence set forth in SEQ ID NO: 20 (GQNX₄TSQTSSPS) wherein X₄ is selected from the group consisting of A, G, I, L, and V, optionally wherein X₄ is A;
(c) the amino acid sequence set forth in SEQ ID NO: 21 (GQNDTSX₇TSSPS) wherein X₇ is selected from the group consisting of A, G, I, L, and V, optionally wherein X₇ is A;
(d) the amino acid sequence set forth in SEQ ID NO: 22 (GQNDTSQX₈SSPS) wherein X₈ is selected from the group consisting of A, G, I, L, and V, optionally wherein X₈ is A;
(e) the amino acid sequence set forth in SEQ ID NO: 23 (GQNDTSQX₈X₉SPS), wherein each of X₈ and X₉ is independently selected from the group consisting of A, G, I, L, and V, optionally wherein each of X₈ and X₉ is A;
(f) the amino acid sequence set forth in SEQ ID NO: 24 (GQNDTSQX₈SX₁₀PS), wherein each of X₈ and X₁₀ is independently selected from the group consisting of A, G, I, L, and V, optionally wherein each of X₈ and X₁₀ is A
(g) the amino acid sequence set forth in SEQ ID NO: 25 (GQNDTSQX₈X₉X₁₀PS), wherein each of X₈, X₉, and X₁₀ is independently selected from the group consisting of A, G, I, L, and V, optionally wherein each of X₈, X₉, and X₁₀ is A;
(h) the amino acid sequence set forth in SEQ ID NO: 26 (GX₂NDTSQX₈X₉SPS), wherein each of X₂, X₈, and X₉ is independently selected from the group consisting of A, G, I, L, and V, optionally wherein each of X₂, X₈, and X₉ is A;
(i) the amino acid sequence set forth in SEQ ID NO: 27 (GX₂NDTSQX₈SX₁₀PS), wherein each of X₂, X₈, and X₁₀ is independently selected from the group consisting of A, G, I, L, and V, optionally wherein each of X₂, X₈, and X₁₀ is A;
(i) the amino acid sequence set forth in SEQ ID NO: 28 (GQNDTSX₇X₈X₉SPS), wherein each of X₇, X₈, and X₉ is independently selected from the group consisting of A, G, I, L, and V, optionally wherein each of X₇, X₈, and X₉ is A; or
(k) the amino acid sequence set forth in SEQ ID NO: 29 (GQNDTSX₇X₈SX₁₀PS), wherein each of X₇, X₈, and X₁₀ is independently selected from the group consisting of A, G, I, L, and V, optionally wherein each of X₇, X₈, and X₁₀ is A.

53-73. (canceled)

74. The method of claim 1, wherein at least a first unique cell surface marker polypeptide comprises a variant human CD52
(a) comprising the amino acid sequence set forth in SEQ ID NO: 19 (GX₂NDTSQTSSPS) wherein X₂ is selected from the group consisting of A, G, I, L, and V; and at least a second unique cell surface marker polypeptide comprises a variant human CD52 comprising an amino acid sequence selected from the group consisting of SEQ ID NO: 20 (GQNX₄TSQTSSPS) and SEQ ID NO: 21 (GQNDTSX₇TSSPS) wherein each of X₄ and X₇ is independently selected from the group consisting of A, G, I, L, and V;
(b) comprising the amino acid sequence set forth in SEQ ID NO: 2 (GANDTSQTSSPS); and at least a second unique cell surface marker polypeptide comprises a variant human CD52 comprising an amino acid sequence selected from the group consisting of SEQ ID NO: 4 (GQNATSQTSSPS) and SEQ ID NO: 7 (GQNDTSATSSPS);
(c) consists of a variant human CD52 consisting of the amino acid sequence set forth in SEQ ID NO: 2 (GANDTSQTSSPS); and at least a second unique cell surface marker polypeptide consists of a variant human CD52 consisting of an amino acid sequence selected from the group consisting of SEQ ID NO: 4 (GQNATSQTSSPS) and SEQ ID NO: 7 (GQNDTSATSSPS)
(d) comprises a variant human CD52 comprising the amino acid sequence set forth as SEQ ID NO: 2 (GANDTSQTSSPS); and at least a second unique cell surface marker polypeptide comprises a variant human CD52 comprising the amino acid sequence set forth as SEQ ID NO: 4 (GQNATSQTSSPS);
(e) consists of a variant human CD52 consisting of the amino acid sequence set forth as SEQ ID NO: 2 (GANDTSQTSSPS); and at least a second unique cell surface marker polypeptide consists of a variant human CD52 consisting of the amino acid sequence set forth as SEQ ID NO: 4 (GQNATSQTSSPS);
(f) comprising the amino acid sequence set forth as SEQ ID NO: 2 (GANDTSQTSSPS);
and at least a second unique cell surface marker polypeptide comprises a variant human CD52 comprising the amino acid sequence set forth as SEQ ID NO: 7 (GQNDTSATSSPS);
(g) consists of a variant human CD52 consisting of the amino acid sequence set forth as SEQ ID NO: 2 (GANDTSQTSSPS); and at least a second unique cell surface marker polypeptide consists of a variant human CD52 consisting of the amino acid sequence set forth as SEQ ID NO: 7 (GQNDTSATSSPS);
(h) comprises a variant human CD52 comprising the amino acid sequence set forth as SEQ ID NO: 4 (GQNATSQTSSPS); and at least a second unique cell surface marker polypeptide comprises a variant human CD52 comprising the amino acid sequence set forth as SEQ ID NO: 7 (GQNDTSATSSPS);
(i) comprises a variant human CD52 comprising the amino acid sequence set forth as SEQ ID NO: 20 (GQNX₄TSQTSSPS); at least a second unique cell surface marker polypeptide comprises a variant human CD52 comprising the amino acid sequence set forth as SEQ ID NO: 23 (GX₂NDTSQX₈X₉SPS); and at least a third unique cell surface marker polypeptide comprises a variant human CD52 comprising the amino acid sequence set forth as SEQ ID NO: 28 (GQNDTSX₇X₈X₉SPS), wherein each of X₄, X₇, X₈, and X₉ is independently selected from the group consisting of A, G, I, L, and V;
(j) comprising the amino acid sequence set forth as SEQ ID NO: 20 (GQNX₄TSQTSSPS); at least a second unique cell surface marker polypeptide comprises a variant human CD52 comprising the amino acid sequence set forth as SEQ ID NO: 27 (GX₂NDTSQX₈SX₁₀PS); and at least a third unique cell surface marker polypeptide comprises a variant human CD52 comprising the amino acid sequence set forth as SEQ ID NO: 29 (GQNDTSX₇X₈SX₁₀PS) wherein each of X₄, X₇, X₈, and X₁₀ is independently selected from the group consisting of A, G, I, L, and V;
(k) comprising the amino acid sequence set forth as SEQ ID NO: 4 (GQNATSQTSSPS); at least a second unique cell surface marker polypeptide comprises a variant human CD52 comprising the amino acid sequence set forth as SEQ ID NO: 15 (GANDTSQAASPS); and at least a third unique cell surface marker polypeptide comprises a variant human CD52 comprising the amino acid sequence set forth as SEQ ID NO: 17 (GQNDTSAAASPS); or
(l) comprises a variant human CD52 comprising the amino acid sequence set forth as SEQ ID NO: 4 (GQNATSQTSSPS); at least a second unique cell surface marker polypeptide comprises a variant human CD52 comprising the amino acid sequence set forth as SEQ ID NO: 16 (GANDTSQASAPS); and at least a third unique cell surface marker polypeptide comprises a variant human CD52 comprising the amino acid sequence set forth as SEQ ID NO: 18 (GQNDTSAASAPS).

75-86. (canceled)

87. The method of claim 1, wherein at least one unique target polypeptide comprises a therapeutic polypeptide or a therapeutic protein, optionally wherein
at least one unique target polypeptide is a polypeptide of a multi-chain protein, wherein each unique target polypeptide is a polypeptide of a multi-chain protein,
at least one unique target polypeptide is a polypeptide of an antibody,
each unique target polypeptide is a polypeptide of an antibody,
at least one unique target polypeptide is a polypeptide of a crossover dual-variable domain (CODV) Ig-like protein,
each unique target polypeptide is a polypeptide of a crossover dual-variable domain (CODV) Ig-like protein,
at least one unique target polypeptide is a polypeptide of a CODV triabody, or
each unique target polypeptide is a polypeptide of a CODV triabody.

88-95. (canceled)

96. The method of claim 1, wherein the recombinant mammalian host cell is a CHO cell.

97. An engineered mammalian host cell comprising a plurality of recombinant polynucleotides, wherein each recombinant polynucleotide comprises a promoter, a nucleotide sequence encoding a unique cell surface marker polypeptide, and a nucleotide sequence encoding a unique target polypeptide, wherein both the nucleotide sequence encoding the unique cell surface marker polypeptide and the nucleotide sequence encoding the unique target polypeptide are transcribed on the same mRNA.

98. The engineered mammalian host cell of claim 97, wherein the plurality of unique target polypeptides comprises 2 to 8 unique target polypeptides.

99-102. (canceled)

103. The engineered mammalian host cell claim 97, wherein each recombinant polynucleotide comprises, from 5′ to 3′, the promoter, the nucleotide sequence encoding the unique cell surface marker polypeptide, and the nucleotide sequence encoding the unique target polypeptide;
each recombinant polynucleotide comprises 1 to 4 promoters, 1 to 4 nucleotide sequences each encoding a unique cell surface marker polypeptide, and 1 to 4 nucleotide sequences each encoding a unique target polypeptide, wherein one of the nucleotide sequences encoding a unique cell surface marker polypeptide is transcribed on the same mRNA as one of the nucleotide sequences encoding a unique target polypeptide;
each recombinant polynucleotide is a bicistronic polynucleotide comprising two promoters and two nucleotide sequences each encoding a unique target polypeptide, and further comprising one or two nucleotide sequences each encoding a unique cell surface marker polypeptide;
each recombinant polynucleotide is a bicistronic polynucleotide comprising two promoters and two nucleotide sequences each encoding a unique target polypeptide, and further comprising one or two nucleotide sequences each encoding a unique cell surface marker polypeptide;
each bicistronic polynucleotide comprises a single nucleotide sequence encoding a unique cell surface marker polypeptide;
each bicistronic polynucleotide comprises two nucleotide sequences each encoding a unique cell surface marker polypeptide, and wherein each one of the nucleotide sequences encoding a unique cell surface marker polypeptide is transcribed on the same mRNA as one of the nucleotide sequences encoding a unique target polypeptide;
each recombinant polynucleotide comprises, from 5′ to 3′, one of the from 1-4 promoters, one of the from 1-4 nucleotide sequences encoding a unique cell surface marker polypeptide, and one of the from 1-4 nucleotide sequences encoding a unique target polypeptide;
each recombinant polynucleotide comprises one promoter, one nucleotide sequence encoding a unique cell surface marker polypeptide, and one nucleotide sequence encoding a unique target polypeptide, wherein the nucleotide sequence encoding a unique cell surface marker polypeptide is transcribed on the same mRNA as the nucleotide sequence encoding a unique target polypeptide;
each recombinant polynucleotide further comprises an internal ribosome entry site (IRES) located 3′ to the nucleotide sequence encoding the unique cell surface marker polypeptide and 5′ to the nucleotide sequence encoding the unique target polypeptide; and/or
each recombinant polynucleotide further comprises an alternate (non-ATG) start codon for translation initiation of the nucleotide sequence encoding the unique cell surface marker polypeptide and an ATG start codon for translation initiation of the nucleotide sequence encoding the unique target polypeptide, optionally wherein each alternate (non-ATG) start codon is selected independently from the group consisting of CTG, GTG, TTG, ATT, ATA, and ACG, each alternate (non-ATG) start codon is selected independently from the group consisting of GTG and TTG, or wherein the nucleotide sequence encoding the unique cell surface marker polypeptide is devoid of any ATG triplets.

104-114. (canceled)

115. The engineered mammalian host cell of claim 97, wherein
at least a first recombinant polynucleotide comprises, from 5′ to 3′, a first promoter, a nucleotide sequence encoding a first unique cell surface marker polypeptide, and a nucleotide sequence encoding a first unique target polypeptide; optionally wherein
the first recombinant polynucleotide further comprises an internal ribosome entry site (IRES) located 3′ to the nucleotide sequence encoding the first unique cell surface marker polypeptide and 5′ to the nucleotide sequence encoding the first unique target polypeptide, or the first recombinant polynucleotide further comprises an alternate (non-ATG) start codon for translation initiation of the nucleotide sequence encoding the first unique cell surface marker polypeptide and an ATG start codon for translation initiation of the nucleotide sequence encoding the first unique target polypeptide; and
at least a second recombinant polynucleotide comprises, from 5′ to 3′, a second promoter, a nucleotide sequence encoding a second unique target polypeptide, and a nucleotide sequence encoding a second unique cell surface marker polypeptide, optionally wherein
the second recombinant polynucleotide further comprises an internal ribosome entry site (IRES) located 3′ to the nucleotide sequence encoding the second unique target polypeptide and 5′ to the nucleotide sequence encoding the second unique cell surface marker polypeptide; and/or
wherein each recombinant polynucleotide comprises, from 5′ to 3′, the promoter, the nucleotide sequence encoding the unique target polypeptide, and the nucleotide sequence encoding the unique cell surface marker polypeptide, optionally wherein each recombinant polynucleotide further comprises an internal ribosome entry site (IRES) located 3′ to the nucleotide sequence encoding the unique target polypeptide and 5′ to the nucleotide sequence encoding the unique cell surface marker polypeptide.

116-127. (canceled)

128. The engineered mammalian host cell of claim 97, wherein at least a first unique cell surface marker polypeptide is selected from the group consisting of CD20, CD52, CD59, and variants thereof
at least a second unique cell surface marker polypeptide is selected from the group consisting of CD20, CD52, CD59, and variants thereof;
each unique cell surface marker polypeptide is selected from the group consisting of CD20, CD52, CD59, and variants thereof;
at least a first unique cell surface marker polypeptide is selected from the group consisting of human CD52 and variants thereof;
at least a second unique cell surface marker polypeptide is selected from the group consisting of human CD52 and variants thereof;
each unique cell surface marker polypeptide is selected from the group consisting of human CD52 and variants thereof;
at least a first unique cell surface marker polypeptide is human CD52; and/or
at least a second unique cell surface marker polypeptide is human CD52.

129-130. (canceled)

131. The engineered mammalian host cell of claim 97, wherein at least a first unique cell surface marker polypeptide is a variant of CD52 comprising:
(a) the amino acid sequence set forth in SEQ ID NO: 19 (GX₂NDTSQTSSPS) wherein X₂ is selected from the group consisting of A, G, I, L, and V, optionally wherein X₂ is A;
(b) the amino acid sequence set forth in SEQ ID NO: 20 (GQNX₄TSQTSSPS) wherein X₄ is selected from the group consisting of A, G, I, L, and V, optionally wherein X₄ is A;
(c) the amino acid sequence set forth in SEQ ID NO: 21 (GQNDTSX₇TSSPS) wherein X₇ is selected from the group consisting of A, G, I, L, and V, optionally wherein X₇ is A;
(d) the amino acid sequence set forth in SEQ ID NO: 22 (GQNDTSQX₈SSPS) wherein X₈ is selected from the group consisting of A, G, I, L, and V, optionally wherein X₈ is A;
(e) the amino acid sequence set forth in SEQ ID NO: 23 (GQNDTSQX₈X₉SPS), wherein each of X₈ and X₉ is independently selected from the group consisting of A, G, I, L, and V, optionally wherein each of X₈ and X₉ is A;
(f) the amino acid sequence set forth in SEQ ID NO: 24 (GQNDTSQX₈SX₁₀PS), wherein each of X₈ and X₁₀ is independently selected from the group consisting of A, G, I, L, and V, optionally wherein each of X₈ and X₁₀ is A;
(g) the amino acid sequence set forth in SEQ ID NO: 25 (GQNDTSQX₈X₉X₁₀PS), wherein each of X₈, X₉, and X₁₀ is independently selected from the group consisting of A, G, I, L, and V, optionally wherein each of X₈, X₉, and X₁₀ is A;
(h) the amino acid sequence set forth in SEQ ID NO: 26 (GX₂NDTSQX₈X₉SPS), wherein each of X₂, X₈, and X₉ is independently selected from the group consisting of A, G, I, L, and V, optionally wherein each of X₂, X₈, and X₉ is A;
(i) the amino acid sequence set forth in SEQ ID NO: 27 (GX₂NDTSQX₈SX₁₀PS), wherein each of X₂, X₈, and X₁₀ is independently selected from the group consisting of A, G, I, L, and V, optionally wherein each of X₂, X₈, and X₁₀ is A;
(j) the amino acid sequence set forth in SEQ ID NO: 28 (GQNDTSX₇X₈X₉SPS), wherein each of X₇, X₈, and X₉ is independently selected from the group consisting of A, G, I, L, and V, optionally wherein each of X₇, X₈, and X₉ is A; or
(k) the amino acid sequence set forth in SEQ ID NO: 29 (GQNDTSX₇X₈SX₁₀PS), wherein each of X₇, X₈, and X₁₀ is independently selected from the group consisting of A, G, I, L, and V, optionally wherein each of X₇, X₈, and X₁₀ is A.

132-138. (canceled)

139. The engineered mammalian host cell of claim 97, wherein at least one unique cell surface marker polypeptide is a variant human CD52 comprising:
(a) the amino acid sequence set forth in SEQ ID NO: 19 (GX₂NDTSQTSSPS) wherein X₂ is selected from the group consisting of A, G, I, L, and V
(b) comprising the amino acid sequence set forth in SEQ ID NO: 2 (GANDTSQTSSPS); and at least a second unique cell surface marker polypeptide comprises a variant human CD52 comprising an amino acid sequence selected from the group consisting of SEQ ID NO: 4 (GQNATSQTSSPS) and SEQ ID NO: 7 (GQNDTSATSSPS);
(c) consists of a variant human CD52 consisting of the amino acid sequence set forth in SEQ ID NO: 2 (GANDTSQTSSPS); and at least a second unique cell surface marker polypeptide consists of a variant human CD52 consisting of an amino acid sequence selected from the group consisting of SEQ ID NO: 4 (GQNATSQTSSPS) and SEQ ID NO: 7 (GQNDTSATSSPS)
(d) comprises a variant human CD52 comprising the amino acid sequence set forth as SEQ ID NO: 2 (GANDTSQTSSPS); and at least a second unique cell surface marker polypeptide comprises a variant human CD52 comprising the amino acid sequence set forth as SEQ ID NO: 4 (GQNATSQTSSPS);
(e) consists of a variant human CD52 consisting of the amino acid sequence set forth as SEQ ID NO: 2 (GANDTSQTSSPS); and at least a second unique cell surface marker polypeptide consists of a variant human CD52 consisting of the amino acid sequence set forth as SEQ ID NO: 4 (GQNATSQTSSPS);
(f) comprising the amino acid sequence set forth as SEQ ID NO: 2 (GANDTSQTSSPS); and at least a second unique cell surface marker polypeptide comprises a variant human CD52 comprising the amino acid sequence set forth as SEQ ID NO: 7 (GQNDTSATSSPS);
(g) consists of a variant human CD52 consisting of the amino acid sequence set forth as SEQ ID NO: 2 (GANDTSQTSSPS); and at least a second unique cell surface marker polypeptide consists of a variant human CD52 consisting of the amino acid sequence set forth as SEQ ID NO: 7 (GQNDTSATSSPS);
(h) comprises a variant human CD52 comprising the amino acid sequence set forth as SEQ ID NO: 4 (GQNATSQTSSPS); and at least a second unique cell surface marker polypeptide comprises a variant human CD52 comprising the amino acid sequence set forth as SEQ ID NO: 7 (GQNDTSATSSPS);
(i) comprises a variant human CD52 comprising the amino acid sequence set forth as SEQ ID NO: 20 (GQNX₄TSQTSSPS); at least a second unique cell surface marker polypeptide comprises a variant human CD52 comprising the amino acid sequence set forth as SEQ ID NO: 23 (GX₂NDTSQX₈X₉SPS); and at least a third unique cell surface marker polypeptide comprises a variant human CD52 comprising the amino acid sequence set forth as SEQ ID NO: 28 (GQNDTSX₇X₈X₉SPS), wherein each of X₄, X₇, X₈, and X₉ is independently selected from the group consisting of A, G, I, L, and V;
(i) comprising the amino acid sequence set forth as SEQ ID NO: 20 (GQNX₄TSQTSSPS); at least a second unique cell surface marker polypeptide comprises a variant human CD52 comprising the amino acid sequence set forth as SEQ ID NO: 27 (GX₂NDTSQX₈SX₁₀PS); and at least a third unique cell surface marker polypeptide comprises a variant human CD52 comprising the amino acid sequence set forth as SEQ ID NO: 29 (GQNDTSX₇X₈SX₁₀PS) wherein each of X₄, X₇, X₈, and X₁₀ is independently selected from the group consisting of A, G, I, L, and V;
(k) comprising the amino acid sequence set forth as SEQ ID NO: 4 (GQNATSQTSSPS); at least a second unique cell surface marker polypeptide comprises a variant human CD52 comprising the amino acid sequence set forth as SEQ ID NO: 15 (GANDTSQAASPS); and at least a third unique cell surface marker polypeptide comprises a variant human CD52 comprising the amino acid sequence set forth as SEQ ID NO: 17 (GQNDTSAAASPS); or
(l) comprises a variant human CD52 comprising the amino acid sequence set forth as SEQ ID NO: 4 (GQNATSQTSSPS); at least a second unique cell surface marker polypeptide comprises a variant human CD52 comprising the amino acid sequence set forth as SEQ ID NO: 16 (GANDTSQASAPS); and at least a third unique cell surface marker polypeptide comprises a variant human CD52 comprising the amino acid sequence set forth as SEQ ID NO: 18 (GQNDTSAASAPS).

140-173. (canceled)

174. The engineered mammalian host cell of claim 97, wherein at least one unique target polypeptide comprises a therapeutic polypeptide or a therapeutic protein, optionally wherein
at least one unique target polypeptide is a polypeptide of a multi-chain protein, wherein each unique target polypeptide is a polypeptide of a multi-chain protein,
at least one unique target polypeptide is a polypeptide of an antibody,
each unique target polypeptide is a polypeptide of an antibody,
at least one unique target polypeptide is a polypeptide of a crossover dual-variable domain (CODV) Ig-like protein,
each unique target polypeptide is a polypeptide of a crossover dual-variable domain (CODV) Ig-like protein,
at least one unique target polypeptide is a polypeptide of a CODV triabody, or
each unique target polypeptide is a polypeptide of a CODV triabody.

175-183. (canceled)

184. An isolated variant human CD52 comprising an amino acid sequence selected from the group consisting of SEQ ID NO: 19 (GX₂NDTSQTSSPS) wherein X₂ is selected from the group consisting of A, G, I, L, and V;
SEQ ID NO: 20 (GQNX₄TSQTSSPS) wherein X₄ is selected from the group consisting of A, G, I, L, and V; or
SEQ ID NO: 22 (GQNDTSQX₈SSPS) wherein X₈ is selected from the group consisting of A, G, I, L, and V.

185-189. (canceled)
